(19) United States
(12) Patent Application Publication
(10) Pub. No.: US 2022/0354764 A1
(43) Pub. Date: Nov. 10, 2022

(54) METHODS FOR PROMOTING HAIR GROWTH

(71) Applicant: Follica, Inc., Boston, MA (US)

(72) Inventors: Jonathan Lee Bissett, Somerville, MA (US); Jason Venkat Bhardwaj, Newton, MA (US); David Paul Chastain, Boston, MA (US); Ken Washenik, Los Angeles, CA (US)

(21) Appl. No.: 17/059,124

(22) PCT Filed: May 30, 2019
(86) PCT No.: PCT/US19/34702

§ 371 Date: Nov. 25, 2020

Related U.S. Application Data
(60) Provisional application No. 62/678,068, filed on May 30, 2018.
(60) Provisional application No. 62/678,129, filed on May 30, 2018.
(60) Provisional application No. 62/730,831, filed on Sep. 13, 2018.

(51) Int. Cl. A61K 8/49; A61P 17/14; A61Q 7/00

(52) U.S. Cl. CPC A61K 8/4953; A61P 17/14; A61Q 7/00; A61K 2800/91; A61M 2037/0023

(57) ABSTRACT

Methods for promoting hair growth, and needling devices for use with such methods, are provided. Certain of the methods include disruption of the skin with a powered needling device, followed by an administration of a hair growth-promoting agent.

This application is a National Stage Entry of International Patent Application No. PCT/US2019/034702, filed May 30, 2019, which claims priority to U.S. Provisional Application No. 62/678,068, filed May 30, 2018, U.S. Provisional Application No. 62/678,129, filed May 30, 2018, and U.S. Provisional Application No. 62/730,831, filed Sep. 13, 2018, each of which is hereby incorporated by reference in its entirety for all purposes.

### TECHNICAL FIELD

Methods for promoting hair growth are generally described.

### BACKGROUND

Current treatments of hair loss in human subjects involve a limited selection of agents and regimens, such as chemical and surgical approaches that either stimulate or transplant pre-existing hair.

Chemical treatments involve the use of drugs for the treatment of certain types of hair loss. Use of these types of treatments alone are typically insufficiently effective in both preventing hair loss and stimulating the growth of significant terminal hair in the scalp of after baldness has advanced. Use of these drugs alone do not create hair follicles of the kind that were there before balding, and the resultant hair follicles are smaller and the scalp has less density of terminal hairs.

More drastic measures for treating hair loss involve hair transplantation—in which scalp strips, hair follicles or follicular units from the occipital scalp (which are resistant to the effects of androgens in inducing androgenetic alopecia) are excised and transplanted to a person's balding or thinning areas.

Another surgical method that has been used is scalp reduction: in this procedure, the skin in the balding area of the scalp is surgically excised and the surrounding skin (with hair) is pulled together and sutured. Surgical methods are less effective for diffuse hair loss and are less effective for women. Additionally, younger patients are not ideal candidates for surgical methods because the pattern and extent of future hair loss is variable. For all patients, hair transplantation can be inconvenient because of the invasive nature of the surgery, recovery time, length of time required before showing a cosmetic effect, creation of scarring, and expense. Furthermore, cosmetic coverage is constrained by the area of and the number of hairs in a patient's donor sites.

Androgenetic alopecia (“AGA”) is a progressive condition in which hair follicles gradually miniaturize and produce shorter, finer, hairs. The ratio of non-vellus to vellus hairs is reduced from 7:1 to less than 2:1. Ultimately, hair follicles go dormant until eventual death in advanced stages of the disorder. AGA is a genetically-mediated disorder which occurs in both men and women (also known as male or female pattern hair loss). In women, the histological features of the condition are the same as in men, but susceptibility, age at onset, rate of progression and pattern of hair loss differ between genders. AGA affects approximately 50% of men by the age of 50 years and approximately 50% of women by the age of 80 years. Common male pattern baldness (MPB) accounts for more than 95% of hair loss in men. By the age of thirty-five two-thirds of American men will experience some degree of appreciable hair loss, and by the age of fifty approximately 85% of men have significantly thinning hair. Female pattern baldness affects approximately 50% of women by the age of 80 years. The prevalence increases with age from approximately 12% amongst women aged between 20 and 29 years to over 50% of women over the age of 80 (Gan and Sinclair 2005). Presently, treatment of AGA involves medication, or transplantation of existing hair follicles. The currently available medications, which include topically administered and orally administered medications, require continuous treatment for lasting effect.

Primary cicatricial alopecia (PCA) disorders are currently treated as inflammatory disorders. Patients with lymphocytic PCA are typically prescribed oral, topical or intralesional injections of anti-inflammatory drugs. Oral drugs include hydroxychloroquine, doxycycline, mycophenolate mofetil, cyclosporine, or corticosteroids; topical drugs include corticosteroids, topical tacrolimus, or topical pimecrolimus; and triamcinolone acetonide is used as an injected drug. Antimicrobial drugs are prescribed for neutrophilic (neutrophil-mediated) PCA after culture and sensitivities direct the appropriate selection. Dissecting cellulitis, with its mixed infiltrate, responds to isotretinoin treatment. None of these treatments is curative and, at best, the symptoms are arrested and clinical signs resolve. Hair loss often continues slowly.

Lichen planopilaris (LPP) is a primary cicatricial (scarring) alopecia of the scalp that is difficult to diagnose and treat and can result in permanent hair loss. The diagnosis of LPP is made by taking into account the clinical presentation, histologic findings, and evaluation by dermatoscopy or trichoscopy. Patients often have irregular patches of hair loss with associated perifollicular erythema, scalp tenderness, and scale. This can progress to permanent loss of scalp hair with loss of the follicular ostia noted on clinical examination. Lesions may be burning, pruritic, ulcerated or atrophic.

The pathogenesis of LPP remains unclear, however, the concept of loss of immune privilege playing a key role in the pathogenesis of LPP has been explored. Immune privilege may protect from autoimmune attack or by suppressing antigen presentation in the hair bulge region where most of the lymphocytic infiltrate is centered.

Although various medical therapies and modalities have been tried off-label, LPP is often recalcitrant to treatment. High potency topical corticosteroids or intralesional corticosteroids are often used as first-line therapies, with some improvement shown using these treatments. However, such therapies have been associated with high rates of relapse. Patients who have extensive or rapidly progressive disease are often treated with systemic therapy, such as hydroxychloroquine and tetracyclines, or systemic immunosuppressants such as myophenolate mofetil and cyclosporine. These therapies are associated with significant side effects.

Because of limited effective treatment options of hair loss, the inventors have recognized the need for novel, safe and effective treatments for hair loss, including those that lead to hair follicle neogenesis, resulting in visible hair.

### SUMMARY

Described herein are methods for promoting hair growth, and/or promoting hair follicle development and/or promoting hair follicle neogenesis, and/or promoting activation on an area of the skin of a subject by disrupting the affected area of the skin, e.g., via integumental pertubation, and subsequent application of pharmaceutical compositions, e.g., hair growth agent, to the disrupted area of the skin. Such methods can be used to treat conditions associated with hair loss, e.g., baldness, including both male and female pattern baldness, alopecia, including AGA, lichen planopilaris (LPP), Primary cicatricial alopecia (PCA), and other conditions associated with hair loss.

In some embodiments, the method for stimulating hair growth in an area of skin with hair loss comprises disrupting the skin on a weekly basis. In one embodiment, the method for stimulating hair growth in an area of skin with hair loss comprises: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i).

In some embodiments, the reciprocating needles are microneedles. In some embodiments, the needling device is a device having the features of any of the devices described herein. In some embodiments, the needling device is a microneedling device such as the microneedling device described herein and in U.S. Published Application No. 2018/0280675.

In some embodiments, the skin disrupting in step (i) is achieved with the needling device by applying a needle strike density of about 1400 to 1800 needle strikes per cm\hat{ }2. In some embodiments, the skin disrupting in step (i) is achieved with the needling device by applying a needle strike density of about 1600 needle strikes per cm\hat{ }2.

In some embodiments, the minoxidil solution in steps (ii) indoor (iii) is topically applied approximately every 12 hours. In some embodiments, the minoxidil solution in step(s) (ii) and/or (iii) is topically applied every 12 hours.

In some embodiments, step (iv) comprises repeating steps (i)-(iii) at least 3 times. In some embodiments, step (iv) comprises repeating steps (i)-(iii) at least 6 times. In some embodiments, step (iv) comprises repeating steps (i)-(iii) at least 12 times. In some embodiments, step (iv) comprises repeating steps (i)-(iii) at least 18 times. In some embodiments, step (iv) comprises repeating steps (i)-(iii) at least 24 times. In some embodiments, step (iv) comprises repeating steps (i)-(iii) at least 30 times. In some embodiments, step (iv) comprises repeating steps (i)-(iii) at least 36 times. In some embodiments, step (iv) comprises repeating steps (i)-(iii) at least 42 times. In some embodiments, step (iv) comprises repeating steps (i)-(iii) at least 48 times.

In some embodiments, step (iv) comprises repeating step (i) 12 to 24 hours following step (iii). In some embodiments, step (iv) comprises repeating step (i) approximately 24 hours following step (iii). In some embodiments, step (iv) comprises repeating step (i) 24 hours following step (iii).

In some embodiments step(s) (ii) and/or (iii) further comprise(s) applying an analgesic solution to the disrupted area of the skin, e.g., any of the analgesic solutions described herein. In some embodiments step(s) (ii) and/or (iii) further comprise(s) applying an antibiotic solution to the disrupted area of the skin, e.g., any of the antibiotic solutions described herein In some embodiments step(s) (ii) and/or (iii) further comprise(s) applying a steroid solution to the disrupted area of the skin, e.g., any of the steroid solutions described herein.

In some embodiments, the method further comprises a step of checking for wound healing after step (i) and prior to step (ii). In some embodiments, confirmation of wound healing is required prior to proceeding with step (ii).

In some embodiments, the method for stimulating hair growth in an area of skin with hair loss comprises disrupting the skin on a bi-weekly basis. In one embodiment, the method for stimulating hair growth in an area of skin with hair loss comprises: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i).

In one embodiment, the method for stimulating hair growth in an area of skin with hair loss comprises: (i) disrupting the skin in an area of hair loss with a needling device having a motor that drives reciprocating needles; (ii) 24 to 36 hours following step (i), topically applying minoxidil solution to the disrupted area of the skin 2 times daily for 6 days; (iii) following step (ii), topically applying minoxidil solution to the disrupted area of the skin 2 times daily for 7 days without disrupting the skin in an area of hair loss with a needling device; and (iv) repeating steps (i)-(iii). In one embodiment, the method for stimulating hair growth in an area of skin with hair loss comprises: (i) disrupting the skin in an area of hair loss with a needling device having a motor that drives reciprocating needles; (ii) approximately 24 hours following step (i), topically applying minoxidil solution to the disrupted area of the skin 2 times daily for 6 days; (iii) following step (ii), topically applying minoxidil solution to the disrupted area of the skin 2 times daily for 7 days without disrupting the skin in an area of hair loss with a needling device; and (iv) repeating steps (i)-(iii). In one embodiment, the method for stimulating hair growth in an area of skin with hair loss comprises: (i) disrupting the skin in an area of hair loss with a needling device having a motor that drives reciprocating needles; (ii) approximately 36 hours following step (i), topically applying minoxidil solution to the disrupted area of the skin 2 times daily for 6 days; (iii) following step (ii), topically applying minoxidil solution to the disrupted area of the skin 2 times daily for 7 days without disrupting the skin in an area of hair loss with a needling device; and (iv) repeating steps (i)-(iii). In one embodiment, the method for stimulating hair growth in an area of skin with hair loss comprises: (i) disrupting the skin in an area of hair loss with a needling device having a motor that drives reciprocating needles; (ii) approximately 48 hours following step (i), topically applying minoxidil solution to the disrupted area of the skin 2 times daily for 6 days; (iii) following step (ii), topically applying minoxidil solution to the disrupted area of the skin 2 times daily for 7 days without disrupting the skin in an area of hair loss with a needling device; and (iv) repeating steps (i)-(iii). In some embodiments, the minoxidil solution in steps (ii) and (ii) is 5% minoxidil. In some embodiments, the minoxidil solution in steps (ii) and (iii) is 2% minoxidil. In some embodiments, in step (ii), 2% minoxidil is applied 2× daily for the first 2 days and 5% minoxidil is applied daily for the subsequent 4 days and in step (iii), 5% minoxidil is applied 2× daily for 7 days.

In some embodiments, the reciprocating needles are microneedles. In some embodiments, the needling device is a device having the features of any of the devices described herein. In some embodiments, the needling device is a microneedling device such as the microneedling device described herein and in U.S. Published Application No. 2018/0280675.

In some embodiments, the skin disrupting in step (i) is achieved with the needling device by applying a needle strike density of about 1400 to 1800 needle strikes per cm\hat{ }2. In some embodiments, the skin disrupting in step (i) is achieved with the needling device by applying a needle strike density of about 1600 needle strikes per cm\hat{ }2.

In some embodiments, the minoxidil solution in step(s) (ii) and/or (iii) is topically applied approximately every 12 hours. In some embodiments, the minoxidil solution in step(s) (ii) and/or (iii) is topically applied every 12 hours.

In some embodiments, step (iv) comprises repeating steps (i)-(iii) at least 3 times. In some embodiments, step (iv) comprises repeating steps (i)-(iii) at least 6 times. In some embodiments, step (iv) comprises repeating steps (i)-(iii) at least 12 times. In some embodiments, step (iv) comprises repeating steps (i)-(iii) at least 18 times. In some embodiments, step (iv) comprises repeating steps (i)-(iii) at least 24 times. In some embodiments, step (iv) comprises repeating steps (i)-(iii) at least 30 times. In some embodiments, step (iv) comprises repeating steps (i)-(iii) at least 36 times. In some embodiments, step (iv) comprises repeating steps (i)-(iii) at least 42 times. In some embodiments, step (iv) comprises repeating steps (i)-(iii) at least 48 times.

In some embodiments, step (iv) comprises repeating step (i) 12 to 24 hours following step (iii). In some embodiments, step (iv) comprises repeating step (i) approximately 24 hours following step (iii). In some embodiments, step (iv) comprises repeating step (i) 24 hours following step (iii).

In some embodiments step(s) (ii) and/or (iii) further comprise(s) applying an analgesic solution to the disrupted area of the skin, e.g., any of the analgesic solutions described herein. In some embodiments step(s) (ii) and/or (iii) further comprise(s) applying an antibiotic solution to the disrupted area of the skin, e.g., any of the antibiotic solutions described herein In some embodiments step(s) (ii) and/or (iii) further comprise(s) applying a steroid solution to the disrupted area of the skin, e.g., any of the steroid solutions described herein.

In some embodiments, the method further comprises a step of checking for wound healing after step (i) and prior to step (ii). In some embodiments, confirmation of wound healing is required prior to proceeding with step (ii).

In some embodiments, the method for stimulating hair growth in an area of skin with hair loss comprises disrupting the skin on a monthly basis. In one embodiment, the method for stimulating hair growth in an area of skin with hair loss comprises: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 24 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In one embodiment, the method for stimulating hair growth in an area of skin with hair loss comprises: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i).

In some embodiments, the reciprocating needles are microneedles. In some embodiments, the needling device is a device having the features of any of the devices described herein. In some embodiments, the needling device is a microneedling device such as the microneedling device described herein and in U.S. Published Application No. 2018/0280675.

In some embodiments, the skin disrupting in step (i) is achieved with the needling device by applying a needle strike density of about 1400 to 1800 needle strikes per cm\hat{ }2. In some embodiments, the skin disrupting in step (i) is achieved with the needling device by applying a needle strike density of about 1600 needle strikes per cm^{\hat{ }2}.

In some embodiments, the minoxidil solution in step(s) (ii) and/or (iii) is topically applied approximately every 12 hours. In some embodiments, the minoxidil solution in step(s) (ii) and/or (iii) is topically applied every 12 hours.

In some embodiments, step (iv) comprises repeating steps (i)-(iii) at least 3 times. In some embodiments, step (iv) comprises repeating steps (i)-(iii) at least 6 times. In some embodiments, step (iv) comprises repeating steps (i)-(iii) at least 12 times. In some embodiments, step (iv) comprises repeating steps (i)-(iii) at least 18 times. In some embodiments, step (iv) comprises repeating steps (i)-(iii) at least 24 times. In some embodiments, step (iv) comprises repeating steps (i)-(iii) at least 30 times. In some embodiments, step (iv) comprises repeating steps (i)-(iii) at least 36 times. In some embodiments, step (iv) comprises repeating steps (i)-(iii) at least 42 times. In some embodiments, step (iv) comprises repeating steps (i)-(iii) at least 48 times.

In some embodiments, step (iv) comprises repeating step (i) 12 to 24 hours following step (iii). In some embodiments, step (iv) comprises repeating step (i) approximately 24 hours following step (iii). In some embodiments, step (iv) comprises repeating step (i) 24 hours following step (iii).

In some embodiments step(s) (ii) and/or (iii) further comprise(s) applying an analgesic solution to the disrupted area of the skin, e.g., any of the analgesic solutions described herein. In some embodiments step(s) (ii) and/or (iii) further comprise(s) applying an antibiotic solution to the disrupted area of the skin, e.g., any of the antibiotic solutions described herein In some embodiments step(s) (ii) and/or (iii) further comprise(s) applying a steroid solution to the disrupted area of the skin, e.g., any of the steroid solutions described herein.

In some embodiments, the method further comprises a step of checking for wound healing after step (i) and prior to step (ii). In some embodiments, confirmation of wound healing is required prior to proceeding with step (ii).

In any of the above-described methods, the disruption of the skin is accomplished using a needling device, wherein the skin is disrupted to a skin depth of about 500-1000 uM. In some embodiments, the skin is disrupted to a skin depth of about 800 uM. In some embodiments, the skin is disrupted to a skin depth of 800 uM.

In any of the above-described methods, the disruption of the skin is accomplished using a needling device, wherein the resultant needle strike density is about 1400 to 1800 needle strikes per cm² area. In some embodiments, the needle strike density is about 1600 needle strikes per cm² area. In some embodiments, the needle strike density is 1600 needle strikes per cm² area.

In any of the above-described methods, the disruption of the skin is accomplished using a needling device having a motor that drives reciprocating needles by applying a needle strike density of about 1400 to 1800 needle strikes per cm² area. In some embodiments, the needle strike density is about 1600 needle strikes per cm² area. In some embodiments, the needle strike density is 1600 needle strikes per cm² area.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm.

In any of the above-described methods, the disruption of the skin is accomplished using a needling device having a needle aray and having a motor that drives reciprocating needles in the needle array, and choosing a setting on the needling device to control reciprocation speed of the needles in the needle array.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm. In some embodiments, the method further comprises choosing a setting on the needling device to control reciprocation speed of the needles in the needle array.

In any of the above-described methods, the disruption of the skin is accomplished using a needling device having a needle aray and having a motor that drives reciprocating needles in the needle array, and choosing a setting on the needling device to control reciprocation frequency of the needles in the needle array. In some embodiments, the frequency of the needle area reciprocation is set at 120 Hz.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm. In some embodiments, the method further comprises choosing a setting on the needling device to control reciprocation frequency of the needles in the needle array. In some embodiments, the frequency of the needle area reciprocation is set at 120 Hz.

In any of the above-described methods, the disruption of the skin is accomplished using a needling device having a needle aray and having a motor that drives reciprocating needles in the needle array, wherein the area of skin disrupted using the needling device includes an area of skin at least 2 cm beyond a leading edge of the area of hair loss.

In any of the above-described methods, the disruption of the skin is accomplished using a needling device having a needle aray and having a motor that drives reciprocating needles in the needle array, wherein the needling device is passed over the area of hair loss at a speed of 2 cm/second.

In any of the above-described methods, the disruption of the skin is accomplished using a needling device having a needle aray and having a motor that drives reciprocating needles in the needle array, wherein the angle orientation of the needle array is perpendicular to the direction of translation.

In any of the above-described methods, the disruption of the skin is accomplished using a needling device having a needle aray and having a motor that drives reciprocating needles in the needle array, wherein the needling device is passed over the area of hair loss such that each pass is contiguous to the prior pass without overlap or gaps.

In any of the above-described methods, the disruption of the skin is accomplished using a needling device having a needle aray and having a motor that drives reciprocating needles in the needle array, wherein the needling device is passed over the area of hair loss in a first series of passes parallel to a first direction and in a second series of passes parallel to a second direction that is different than the first direction. In some embodiments, the first direction is perpendicular to the second direction.

In any of the above-described methods, the disruption of the skin is accomplished using a needling device having a needle aray and having a motor that drives reciprocating needles in the needle array, wherein the needles of the needling device form an array of a plurality of lines of needles, each line having a plurality of needles and each line extending parallel to a first direction, wherein the needling device is passed over the area of hair less in a second direction that is perpendicular to the first direction.

In any of the above-described methods, the mean level of minoxidil in the bloodstream is in the range of 1-4 ng/mL. In any of the above-described methods, the maximal level of minoxidil in bloodstream is in the range of 5-20 ng/mL, below the levels at which cardiovascular effects are observed. In one embodiment, the mean level of minoxidil in the bloodstream is in the range of 1.1 ng/mL. In one embodiment, the maximal level of minoxidil in the bloodstream is is 5.5 ng/ml.

In any of the above-described methods, the method is used to treat male pattern baldness, female pattern baldness, Androgenetic alopecia (“AGA”), Primary cicatricial alopecia (PCA) disorders, and/or Lichen planopilaris (LPP).

In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 4 days; and (iv) following step (iii), repeating steps (i)-(iii), where topical application of minoxidil solution does not occur on the day of step (i).

In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 11 days; and (iv) following step (iii), repeating steps (i)-(iii), where topical application of minoxidil solution does not occur on the day of step (i).

In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 24 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 36 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) 24 to 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i). In some embodiments, the method for stimulating hair growth in an area of skin with hair loss comprises 2% minoxidil solution and 5% minoxidil solution for use in a method of stimulating hair growth in an area of hair loss, wherein said method includes performing: (i) disrupting the skin in an area of hair loss with a needling device having a motor that drives reciprocating needles; (ii) approximately 48 hours following step (i), topically applying 2% minoxidil solution to the disrupted area of the skin 2 times daily for 2 days; (iii) following step (ii), topically applying 5% minoxidil solution to the disrupted area of the skin 2 times daily for 25 days; and (iv) following step (iii), repeating steps (i)-(iii), where topical application of minoxidil solution does not occur on the day of step (i).

In some embodiments, the reciprocating needles are microneedles. In some embodiments, the needling device is a device having the features of any of the devices described herein. In some embodiments, the needling device is a microneedling device such as the microneedling device described herein and in U.S. Published Application No. 2018/0280675.

In some embodiments, the skin disrupting in step (i) is achieved with the needling device by applying a needle strike density of about 1400 to 1800 needle strikes per cm\hat{ }2. In some embodiments, the skin disrupting in step (i) is achieved with the needling device by applying a needle strike density of about 1600 needle strikes per cm\hat{ }2.

In some embodiments, the minoxidil solution in step(s) (ii) and/or (iii) is topically applied approximately every 12 hours. In some embodiments, the minoxidil solution in step(s) (ii) and/or (iii) is topically applied every 12 hours.

In some embodiments, step (iv) comprises repeating steps (i)-(iii) at least 3 times. In some embodiments, step (iv) comprises repeating steps (i)-(iii) at least 6 times. In some embodiments, step (iv) comprises repeating steps (i)-(iii) at least 12 times. In some embodiments, step (iv) comprises repeating steps (i)-(iii) at least 18 times. In some embodiments, step (iv) comprises repeating steps (i)-(iii) at least 24 times. In some embodiments, step (iv) comprises repeating steps (i)-(iii) at least 30 times. In some embodiments, step (iv) comprises repeating steps (i)-(iii) at least 36 times. In some embodiments, step (iv) comprises repeating steps (i)-(iii) at least 42 times. In some embodiments, step (iv) comprises repeating steps (i)-(iii) at least 48 times.

In some embodiments, step (iv) comprises repeating step (i) 12 to 24 hours following step (iii). In some embodiments, step (iv) comprises repeating step (i) approximately 24 hours following step (iii). In some embodiments, step (iv) comprises repeating step (i) 24 hours following step (iii).

In some embodiments step(s) (ii) and/or (iii) further comprise(s) applying an analgesic solution to the disrupted area of the skin, e.g., any of the analgesic solutions described herein. In some embodiments step(s) (ii) and/or (iii) further comprise(s) applying an antibiotic solution to the disrupted area of the skin, e.g., any of the antibiotic solutions described herein In some embodiments step(s) (ii) and/or (iii) further comprise(s) applying a steroid solution to the disrupted area of the skin, e.g., any of the steroid solutions described herein.

In some embodiments, the method further comprises a step of checking for wound healing after step (i) and prior to step (ii). In some embodiments, confirmation of wound healing is required prior to proceeding with step (ii).

In any of the above-described methods, the disruption of the skin is accomplished using a needling device, wherein the skin is disrupted to a skin depth of about 500-1000 uM. In some embodiments, the skin is disrupted to a skin depth of about 800 uM. In some embodiments, the skin is disrupted to a skin depth of 800 uM.

In any of the above-described methods, the disruption of the skin is accomplished using a needling device, wherein the resultant needle strike density is about 1400 to 1800 needle strikes per cm\hat{ }2 area. In some embodiments, the needle strike density is about 1600 needle strikes per cm\hat{ }2 area. In some embodiments, the needle strike density is 1600 needle strikes per cm\hat{ }2 area.

In any of the above-described methods, the disruption of the skin is accomplished using a needling device having a motor that drives reciprocating needles by applying a needle strike density of about 1400 to 1800 needle strikes per cm\hat{ }2 area. In some embodiments, the needle strike density is about 1600 needle strikes per cm\hat{ }2 area. In some embodiments, the needle strike density is 1600 needle strikes per cm\hat{ }2 area.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm.

In any of the above-described methods, the disruption of the skin is accomplished using a needling device having a needle aray and having a motor that drives reciprocating needles in the needle array, and choosing a setting on the needling device to control reciprocation speed of the needles in the needle array.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm. In some embodiments, the method further comprises choosing a setting on the needling device to control reciprocation speed of the needles in the needle array.

In any of the above-described methods, the disruption of the skin is accomplished using a needling device having a needle aray and having a motor that drives reciprocating needles in the needle array, and choosing a setting on the needling device to control reciprocation frequency of the needles in the needle array. In some embodiments, the frequency of the needle area reciprocation is set at 120 Hz.

In any of the above-described methods, the disruption of the skin is accomplished using a needling device having a needle aray and having a motor that drives reciprocating needles in the needle array, and choosing a setting on the needling device to control the depth of the needling array. In some embodiments, the depth of the needling array is fixed at 0.8 mm. In some embodiments, the method further comprises choosing a setting on the needling device to control reciprocation frequency of the needles in the needle array. In some embodiments, the frequency of the needle area reciprocation is set at 120 Hz.

In any of the above-described methods, the disruption of the skin is accomplished using a needling device having a needle aray and having a motor that drives reciprocating needles in the needle array, wherein the area of skin disrupted using the needling device includes an area of skin at least 2 cm beyond a leading edge of the area of hair loss.

In any of the above-described methods, the disruption of the skin is accomplished using a needling device having a needle aray and having a motor that drives reciprocating needles in the needle array, wherein the needling device is passed over the area of hair loss at a speed of 2 cm/second.

In any of the above-described methods, the disruption of the skin is accomplished using a needling device having a needle aray and having a motor that drives reciprocating needles in the needle array, wherein the angle orientation of the needle array is perpendicular to the direction of translation.

In any of the above-described methods, the disruption of the skin is accomplished using a needling device having a needle aray and having a motor that drives reciprocating needles in the needle array, wherein the needling device is passed over the area of hair loss such that each pass is contiguous to the prior pass without overlap or gaps.

In any of the above-described methods, the disruption of the skin is accomplished using a needling device having a needle aray and having a motor that drives reciprocating needles in the needle array, wherein the needling device is passed over the area of hair loss in a first series of passes parallel to a first direction and in a second series of passes parallel to a second direction that is different than the first direction. In some embodiments, the first direction is perpendicular to the second direction.

In any of the above-described methods, the disruption of the skin is accomplished using a needling device having a needle aray and having a motor that drives reciprocating needles in the needle array, wherein the needles of the needling device form an array of a plurality of lines of needles, each line having a plurality of needles and each line extending parallel to a first direction, wherein the needling device is passed over the area of hair less in a second direction that is perpendicular to the first direction.

In any of the above-described methods, the mean level of minoxidil in the bloodstream is in the range of 1-4 ng/mL. In any of the above-described methods, the maximal level of minoxidil in bloodstream is in the range of 5-20 ng/mL, below the levels at which cardiovascular effects are observed. In one embodiment, the mean level of minoxidil in the bloodstream is in the range of 1.1 ng/mL. In one embodiment, the maximal level of minoxidil in the bloodstream is is 5.5 ng/ml.

In any of the above-described methods, the method is used to treat male pattern baldness, female pattern baldness, Androgenetic alopecia (“AGA”), Primary cicatricial alopecia (PCA) disorders, and/or Lichen planopilaris (LPP).

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a perspective view of a first embodiment of a needling device;

FIG. 2 is a is a perspective view of the inner components of the needling device of FIG. 1;

FIG. 3A depicts an illustrative example of a “mowing the lawn” approach, with series of passes in a horizontal direction over a treatment area;

FIG. 3B depicts an illustrative example of another “mowing the lawn” approach, with series of passes in a vertical direction over a treatment area;

FIG. 4 is a diagram of resulting needle strikes using the horizontal and vertical passes of FIGS. 3A and 3B;

FIG. 5 illustrates examples where a needling device is not kept perpendicular relative to the skin (left and right), and an example where the needling device maintains perpendicularity with the skin (middle);

FIG. 6A illustrates an example where a user has implemented a moving start with a needling device;

FIG. 6B illustrates an example where a user has not implemented a moving start with a needling device, resulting in higher needle strike density at the starting area;

FIG. 7 illustrates examples where a user has applied insufficient pressure (left), just the right amount of pressure (middle) and too much pressure (right) to the skin using a needling device;

FIG. 8A illustrates an example where a user translates a needling device in a direction perpendicular to the extension direction of the lines of the needles of the needling device needle array; and

FIG. 8A illustrates an example where a user translates a needling device in a direction parallel to the extension direction of the lines of the needles of the needling device needle array.

### DETAILED DESCRIPTION

Aspects described herein relate to methods for promoting hair growth, and/or promoting hair follicle development and/or promoting hair follicle neogenesis, and/or promoting activation on an area of the skin of a subject (for example, a human) by disrupting the affected area of the skin, e.g., via integumental pertubation, and subsequent application of pharmaceutical compositions, e.g., hair growth agent, to the disrupted area of the skin. Aspects described herein relate to methods of treating conditions associated with hair loss, e.g., treating baldness, including both male and female pattern baldness, treating alopecia, treating lichen planopilaris (LPP), and other conditions associated with hair loss by disrupting the affected area of the skin, e.g., via integumental pertubation, and subsequent application of pharmaceutical compositions, e.g., hair growth agent, to the disrupted area of the skin.

Methods for promoting hair growth described herein include the use of a needling device to produce small wounds in skin at a controllable depth and density of disruption. Without wishing to be bound by theory, the inventors have found that such wounding of the skin by the needling device may induce the growth of new hair follicles in the treated area . The needling device may trigger the dermis' natural wound healing mechanisms in a way that generates creates new follicles in the treated area.

It was believed until quite recently that follicle formation occurs but once in a lifetime (in utero), so that a mammal, and particularly a human, is born with a fixed number of follicles, which does not normally increase thereafter. Despite suggestions of the regenerative capacity of the adult mammalian skin to recreate the embryonic follicle, until recently, follicle neogenesis was not proven because of the lack of tools needed to demonstrate the occurrence or hair follicle neogenesis (see, Argyris et al, 1959, Dev. Biol. 1 : 269-80; Miller, 1973, J. Invest. Dermatol. 58: 1-9; and ligman, 1959, Ann NY Acad Sci 83: 507-511).

It has been proposed, however, that hair follicle neogenesis can be associated with wound healing in animals (e.g., rabbits, mice). See, Stenn & Paus, 2001, Physiol. Revs. 81 :449-494. More recently, a series of murine experiments definitively showed that hair follicle-derived epithelial stem cell progenitors migrate out of the follicle and contribute to the re-epithelialization of injured skin (see, Morris et al, 2004, Nature Biotechnology 22:411-417; Ito et al, 2004, Differentiation 72:548-57; and Ito et al, 2005, Nature Medicine 11 : 1351-1354). In animal studies designed to explore the role of Wnt in hair follicle development, Fathke showed that prolonged activation of Wnt signaling during wound healing in mice resulted in generation of rudiments of hair follicles but did not result in the formation of hair follicles or growth of more hair (Fathke et al, 2006, BMC Cell Biol. 7:4).

As noted by Fathke, cutaneous repair in adult mammals following full thickness wounding is understood to result in scar tissue and the loss of the regenerative capability of the hair follicle. Severe wounds and burns are usually associated with a form of cutaneous repair that results in scar tissue and no hair follicles (see, Fathke et al, 2006, BMC Cell Biol. 7:4). However, in a mouse study, Cotsarelis showed that physically disrupting the skin and existing follicles, in a defined fashion, can lead to follicle neogenesis (Ito et al, 2007, Nature 447:316-321). Cotsarelis showed that following closure of large healed wounds created by full thickness excision (FTE) (1 cm2 square wounds) in mice, new hairs are formed at the center of the wound (Ito et al, 2007, Nature 447:316-321). (Argyris, 1976, Amer J Pathol 83:329-338).

Other preclinical studies have identified a therapeutic window after epithelial disruption where the skin reverts to an embryonic state, allowing manipulation of skin and follicle phenotype by addition of compounds. For example, because new hair patterns after wounding are not predetermined, the regulatory pathways relevant to follicle formation (e.g. Wnt, EGFR) can be influenced dramatically, e.g., to increase the number and size of follicles. See, Ito et al. Nature. 2007; 447(7142):316-320; Fathke et al. BMC Cell Biol. 2006; 7:4; Snippert et al. Science. 2010; 327(5971): 1385-1389.

Motorized devices for performing dermabrasion for skin resurfacing and scar restoration have been around for decades. Over these years, the traditional embodiment of a motorized rotating grinding wheel hasn't changed much. Essentially, when power is applied to an abrasive wheel it grinds off stratum corneum and epidermis and sometimes part of the dermis, until the desired clinical effect is achieved. See, Argyris T S, Am J Pathol. However, one key challenge with these type of devices, which also include dermarollers, is that they are highly dependent on the degree of pressure applied which is difficult to control and as a result highly variable especially regarding depth.

More recently, microneedle rollers have been shown to be a novel and safe device for the treatment of androgenetic alopecia, which induces hair regrowth In addition to various animals studies along these lines, a 12-week long study of weekly dermaroller treatment combined with minoxidil treatment lead to statistically superior results over a group treated with minoxidil alone in promoting hair growth in men with AGA for all 3 primary efficacy measures of hair count and patient/investigator assessment of hair growth/scalp coverage (Dhurat et al. (2013), A randomized evaluator blinded study of effect of microneedling in androgenetic alopecia: A pilot study. Int J Trichology.2013; 5:6-11, and references therein). Similar to other perturbation modalities, microneedle rollers are thought to promote follicle neogenesis through wound healing mechanisms and platelet activation. Stem cells are thought to be activated in the hair bulge area under wound healing conditions, along with the induction of hair growth-related genes, such as VEGF, beta-catenin, and Wnt signaling molecules.

Following-up the wounding process with an application of a pharmaceutical composition configured to promote hair growth may target the new follicles to improve the quality, quantity and persistence of new hair. In animal studies, topical minoxidil shortens telogen (i.e, resting stage of the hair cycle), causing premature entry of resting hair follicles into anagen (i.e, the growth stage of the hair follicle cycle), and it may have a similar action in humans. Minoxidil may also cause prolongation of anagen and increases hair follicle size (Messenger A G, Rundegren J. Minoxidil: Mechanisms of action on hair growth. Br J Dermatol 2004; 150:186-94). Without wishing to be bound by theory, while hair follicle neogenesis is stimulated through integumental perturbation, concomitant minoxidil may function to maintain the hair cycling of, as well as thicken, neogenic hair follicles, thus yielding synergistic results in the overall promotion of hair growth.

The current disclosure is directed to novel methods and treatment regimens to exploit a synergy between a micro-needling strategy using a device which can achieve highly defined and tightly controlled pinpoint wounds combined with topical minoxidil application, to achieve superior efficacy results while maintaining safety of minoxidil treatment. Several elements or parameters are combined in the methods and treatment regimens described herein which are critical to obtain these beneficial outcomes, i.e., improved efficacy over previous studies employing minoxidil alone or in combination with perturbation, e.g., using those dermaroller device as described above. Several unique features of the methods, devices, systems, and treatment regimens described herein contribute overall safety and greater efficiency observed when these methods and protocols are followed.

A first critical element for improved efficacy comprises achieving a productive optimal perturbation using the powered needling device described herein, which can promote optimal hair follicle stimulation. In order to achieve functional perturbation resulting in optimal stimulation of hair growth, the optimal degree and type of disruption per needle strike must be applied, resulting in narrow column of disruption, at perpendicular angle to the skin surface, (puncture effect) with controlled optimal depth. Of note, a powered microneedling device as described herein and used in the methods described herein offers significantly more control of depth than the dermabrasion devices referenced above. Described herein are microneedling devices and methods of using these devices so as to achieve a functional perturbation resulting in optimal conditions to promote hair growth. Such conditions provide sufficient perturbation to optimally stimulate follicle neogenesis, while avoiding over-injury (e.g., scraping effects, too much bleeding, cuts and scrapes, e.g., due to excessive dragging of the needles). Optimized parameters necessary to achieve consistent and optimal results, including but not limited to oscillation rates, device translation rates, device perpendicularity, needle insertion duration, needle insertion depths, and needle strike density, are provided herein. For example, an optimal needle strike density can be achieved by different combinations of parameters such as number of needle strikes, width of the needle array and number of overlapping passes.

Additionally, microneedling devices are provided herein which are designed to achieve the parameters needed for optimal functional perturbation. One non-limiting example of a device, which can be used in the methods described herein and is exemplified in the examples is a device described in U.S. Published Application No. 2018/0280675, which is hereby incorporated by reference in its entirety.

A second critical element for improved efficacy are encompassed in the treatment protocols provided herein, using the device in combination with topical minoxidil, which optimize hair growth results, while maintaining safety of the procedures. For example, treatment protocols are provided herein, in which skin disruptions/integumental perturbations are performed at different intervals and repeated different numbers of times until the desired result is achieved. Non-limiting examples include weekly, biweekly or monthly skin disruptions (e.g., performed by microneedling), followed by post-perturbation minoxidil treatments. In one specific embodiment, the skin disruption/integumental perturbation is performed on a biweekly basis, as described in more detail herein including in the examples, and which is for example more efficacious than weekly microneedling using the device. The number of treatment cycles can be adjusted as needed. In a non-limiting example, treatment cycles are performed over a period of three months or longer.

Additionally, minoxidil treatment protocols are provided herein, which are optimized keeping potential safety concerns in mind, while still achieving optimal efficacy. Such protocol steps include for example waiting until skin disruption is given an opportunity to heal before the first application of minoxidil, and also waiting a specified time after the last minoxidil application before commencing a subsequent microneedling treatment. Additionally, a step-up protocol was devised in which the first doses of minoxidil after treatment are 2% solution, e.g., applied 2 times daily, e.g., for two days. Only then is the dosage increased to 5%, which e.g., can be used two times daily until the end of the treatment cycle. This “ramping up” allows serum levels to increase more gradually, reducing the accumulation of minoxidil in serum to achieve a safety profile in line with the over the counter minoxidil, as shown herein, e.g., in Example 1.

Accordingly, provided herein are methods for promoting hair growth in a human subject, wherein the method comprises: integumental perturbation of an area of interest, e.g., the bald scalp of the human subject; and post-perturbation administering minoxidil or a pharmaceutically acceptable salt, isotopic variant, or solvate thereof (“post-perturbation treatment”). In some embodiments, the integumental perturbation is performed with a micro-needling device, e.g., comprising a motor that drives reciprocating needles, as described in more detail herein infra. In some embodiments, the treatment regimen involves administering minoxidil post-perturbation first at a lower concentration, e.g., 2%, and then at a higher concentration, e.g., 5%. In some embodiments, the treatment regimen involves disrupting the epidermis of the skin in an area of hair loss with a needling device having a motor that drives reciprocating microneedles; then after 24 to 36 hours topically applying 2% minoxidil solution to the disrupted area of the skin 2 time(s) daily for 2 days; and then topically applying 5% minoxidil solution to the disrupted area of the skin 2 time(s) daily for 4 days, 11 days, or 27 days. In some embodiments, these steps are repeated, 1, 2, 3, 4, 5, 6, 7, 8, 9, or 10 times.

Also provided herein are methods of enhancing, stimulating, or increasing hair growth or enhancing or increasing the thickness of hair (in some embodiments collectively referred to herein as “promoting” hair growth or hair thickness) on an area of skin of a subject (e.g., a human), the methods comprising subjecting an affected area of the skin to integumental perturbation in combination with administration of a minoxidil or a pharmaceutically acceptable salt thereof (e.g., a pharmaceutical composition comprising minoxidil or a pharmaceutically acceptable salt thereof). In certain embodiments, the method of treating baldness or alopecia or promoting hair growth or thickness of hair results in formation of new hair follicles (“hair follicle neogenesis”), the formation of neogenic-like hair follicles, activation of existing hair follicles, reorganization of existing hair follicles, an increase in the numbers of vellus hairs, an increase in the numbers of non-vellus hairs (e.g., intermediate or terminal), increase in hair thickness, increase in hair mass and/or an increase in the total number of hair in the treated area.

In one embodiment, the methods described herein promotes growth of hair on an area of skin of a subject. In some embodiments, the methods described herein increases the amount or thickness of hair on a treated area of skin of a subject. In some embodiments, the methods described herein results in an increase in the amount of vellus hair on a treated area of skin of a subject. In some embodiments, the methods described herein results in an increase in the amount of non-vellus hair on a treated area of skin of a subject. In some embodiments, the methods described herein results in an increase in the amount of terminal hair on a treated area of skin of a subject. In some embodiments, the methods described herein results in formation of new hair follicles (“hair follicle neogenesis”) in a treated area of skin of a subject. In certain embodiments, the methods described herein results in an increased number of hair follicles in a treated area of skin of a subject. In particular embodiments, the methods described herein results in formation of new hair follicles with vellus-sized hair shafts (i.e., hair shafts with diameters less than 30 microns in diameter) in a treated area of skin of a subject. In some embodiments, the methods described herein results in an increased number of stimulated and activated hair follicles, such as pre-existing hair follicles, in a treated area of skin of a subject. In particular embodiments, the methods described herein results in an increased number of pre-existing hair follicles with vellus-sized hair shafts in a treated area of skin of a subject. In other particular embodiments, methods described herein results in the presence and/or increased induction of hair follicle neogenesis, and/or initiated and/or increase stimulation, activation, and/or reorganization of follicular structures. In some embodiments, the methods described herein result in newly formed, newly branched and/or newly reorganized hair follicles.

In particular embodiments, a method of integumental perturbation described herein comprises microneedling, using, e.g., a device described herein.

Provided herein are methods for treating an integumentally perturbed area of the skin with a post-perturbation treatment for promoting the growth of hair, including vellus hair or terminal hair. In certain embodiments, the post-perturbation treatment is a topical treatment, e.g., topical minoxidil treatment. Pharmaceutical compositions for use in post-perturbation treatment methods are also provided herein.

In some embodiments, a post-perturbation treatment, e.g., minoxidil treatment, is administered immediately after integumental perturbation. In some embodiments, a post-perturbation treatment is administered a certain period of time after integumental perturbation. In one embodiment, the post-perturbation treatment is administered 1 day after perturbation. In some embodiments, the post-perturbation treatment involves a step-up protocol comprising administration of 2% minoxidil, e.g., for 2 days, followed by at least 4 days of 5% minoxidil.

In one embodiment, a method of integumental perturbation, e.g., microneedling using a device and method described herein, in combination with a post-perturbation treatment, e.g., minoxidil, promotes growth of hair on an area of skin of a subject. In some embodiments, the method provided herein increases the amount or thickness of hair on a treated area of skin of a subject. In some embodiments, the method provided herein results in an increase in the amount of vellus hair on a treated area of skin of a subject. In some embodiments, the method provided herein results in an increase in the amount of terminal hair on a treated area of skin of a subject.

### Patient Populations For Baldness, Alopecia And Hair Growth

A candidate subject for treatment with one or more methods described herein is any subject suffering from hair loss, hair thinning, balding, or who has or has had a disease or condition associated therewith, or who wishes to enhance the growth or thickness of hair.

The subject may be any subject, preferably a human subject, including male, female, intermediate/ambiguous (e.g., XO), and transsexual subjects. In certain embodiments, the subject is a human adolescent. In certain embodiments, the subject is undergoing puberty. In certain embodiments, the subject is a middle-aged adult. In certain embodiments, the subject is a premenopausal adult. In certain embodiments, the subject is undergoing menopause. In certain embodiments, the subject is elderly. In certain embodiments, the subject is a human of 1 year old or less, 2 years old or less, 2 years old, 5 years old, 5 to 10 years old, 10 to 15 years old, e.g., 12 years old, 15 to 20 years old, 20 to 25 years old, 25 to 30 years old, 30 years old or older, 30 to 35 years old, 35 years old or older, 35 to 40 years old, 40 years old or older, 40 to 45 years old, 45 to 50 years old, 50 years old or older, 50 to 55 years old, 55 to 60 years old, 60 years old or older, 60 to 65 years old, e.g., 65 years old, 65 to 70 years old, 70 to 75 years old, 75 to 80 years old, 80 to 85 years old, 85 to 90 years old, 90 to 95 years old or 95 years old or older. In some embodiments, the subject is a male 20 to 50 years old. In some embodiments, the subject is a male 20 to 60 years old. In some embodiments, the subject is a male 30 to 60 years old. In some embodiments, the subject is a male 40 to 60 years old. In some embodiments, the subject is a male or female 12 to 40 years old. In some embodiments, the subject is not a female subject. In some embodiments, the subject is not pregnant or expecting to become pregnant. In some embodiments, the subject is not a pregnant female in the first trimester of pregnancy. In some embodiments, the subject is not breastfeeding.

In one embodiment, the treatment is delivered to an area in which hair growth is desired, for example, the scalp, the face (e.g. , the eyebrow, eyelashes, upper lip, lower lip, chin, cheeks, beard area, or mustache area), or another part of the body, such as, e.g., the chest, abdomen, arms, armpits (site of axillary hair), legs, or genitals. In some embodiments, treatment is delivered to the head. In some embodiments, treatment is delivered to the scalp. In some embodiments, treatment is delivered to a balding scalp. In one embodiment, treatment is not delivered to the face. In some embodiments, hair restoration to a wounded or scarred part of the skin is desired. In one embodiment, the scar is caused by surgery, such as a face lift, skin graft, or hair transplant.

The subject may have a disease or disorder of balding or hair loss (including hair thinning), such as forms of nonscarring (noncicatricial) alopecia, such as androgenetic alopecia (AGA), including MPHL or FPHL (e.g., thinning of the hair, i.e., diffuse hair loss in the frontal/parietal scalp), or any other form of hair loss caused by androgens, toxic alopecia, alopecia areata (including alopecia universalis), scarring (cicatricial) alopecia, pathologic alopecia (caused by, e.g., medication, trauma stress, autoimmune diseases, malnutrition, or endocrine dysfunction), trichotillomania, a form of hypotrichosis, such as congenital hypotrichosis, or lichen planopilaris, or any other condition of hair loss or balding known in the art or described infra.

In some embodiments, the subject has hair loss caused by a genetic or hereditary disease or disorder, such as androgenetic alopecia.

In some embodiments, the subject has hair loss caused by anagen effluvium, such as occurs during chemotherapy (with, e.g., 5-fluorouracil, methotrexate, cyclophosphamide, vincristine). In addition to chemotherapy drugs, Anagen effluvium can be caused by other toxins, radiation exposure (including radiation overdose), endocrine diseases, trauma, pressure, and certain diseases, such as alopecia areata (an autoimmune disease that attacks anagen follicles.)

In some embodiments, the subject has hair loss caused by telogen effluvium. Telogen effluvium is caused frequently by drugs like lithium and other drugs like valproic acid and carbamazepine. In addition to psychiatric drugs, telogen effluvium can be induced by childbirth, traction, febrile illnesses, surgery, stress, or poor nutrition. (See, Mercke et al., 2000, Ann. Clin. Psych. 12:35-42).

In some embodiments, the subject has hair loss caused by or associated with medication, such as chemotherapy (e.g. , anti-cancer therapy or cytotoxic drugs), thallium compounds, vitamins (e.g., vitamin A), retinoids, anti-viral therapy, or psychological therapy, radiation (such as the banding pattern of scalp hair loss that may be caused by radiation overdose), trauma, endocrine dysfunction, surgery, physical trauma, x-ray atrophy, burning or other injury or wound, stress, aging, an autoimmune disease or disorder, malnutrition, an infection (such as, e.g., a fungal, viral, or bacterial infection, including chronic deep bacterial or fungal infections), dermatitis, psoriasis, eczema, pregnancy, allergy, a severe illness (e.g., scarlet fever), myxedema, hypopituitarism, early syphilis, discoid lupus erythematosus, cutaneous lupus erythematosus, lichen planus, deep factitial ulcer, granuloma (e.g., sarcoidosis, syphilitic gummas, TB), inflamed tinea capitis (kerion, favus), a slow-growing tumor of the scalp or other skin tumor, or any other disease or disorder associated with or that causes balding or hair loss known in the art or described infra.

In some embodiments, the subject has hair thinning, or “shock loss,” or a bald patch caused by prior use as a source of tissue or follicles for hair transplantation or follicular unit transplantation.

In some embodiments, a candidate subject is a subject who wishes to enhance hair growth, for example, to have more hair, faster-growing hair, longer hair, and/or thicker hair. In some embodiments, the candidate is a subject who wishes to increase hair pigmentation. In some embodiments, the subject is not affected by a condition of excessive hair loss.

In some embodiments, the subject has scarring (cicatricial) alopecia. Forms of cicatricial alopecia that may be treated in accordance with the methods described herein include primary cicatricial alopecia (PCA) and secondary cicatricial alopecia. Primary cicatricial alopecias that may be treated in accordance with the methods described herein include lymphocyte-mediated PCAs, such as lichen planopilaris (LPP), frontal fibrosing alopecia (FFA), central centrifugal cicatricial alopecia (CCCA), and pseudopelade (Brocq); neutrophil-mediated PCAs, such as folliculitis decalvans and tufted folliculitis; and PCAs involving a mixed inflammatory infiltrate, such as occurs in dissecting cellulitis and folliculitis keloidalis.

In some embodiments, a method described herein is used to enhance hair growth in a patient with scarring alopecia. In some embodiments, the patient has a secondary cicatricial alopecia. In some embodiments, the patient has a form of primary cicatricial alopecia, such as lymphocyte-mediated PCAs, such as lichen planopilaris (LPP), frontal fibrosing alopecia (FFA), central centrifugal cicatricial alopecia (CCCA), and pseudopelade (Brocq); neutrophil-mediated PCAs, such as folliculitis decalvans and tufted folliculitis; and PCAs involving a mixed inflammatory infiltrate, such as occurs in dissecting cellulitis and folliculitis keloidalis.

Cicatricial alopecias affect both men and women, most commonly adults, although all ages may be affected. In general, they are rare. There have been a few reports of cicatricial alopecia occurring in a family. However, the majority of patients with cicatricial alopecia have no family history of a similar condition. Lichen planopilaris may affect middle-aged women most commonly. Central centrifugal alopecia may affect black women most commonly. Frontal fibrosing alopecia is seen most commonly in post-menopausal women. Thus, in certain embodiments, in addition to the subjects described herein, a candidate subject for treatment for scarring alopecia is a black woman (e.g., of African-American descent), a middle-aged woman, or a post-menopausal woman.

### Androgenetic Alopecia

Both males and females develop diffuse hair loss in the frontal/parietal scalp called “thinning,” which begins between 12 and 40 years of age. In females, thinning is known as “Female Pattern Hair Loss (FPHL)” and is caused or exacerbated by androgens. (Price VH, 2003, J Investig Dermatol Symp Proc. 8(1):24-7, Androgenetic alopecia in women).

### Male Pattern Hair Loss (Mphl)

After puberty, males begin to lose the scalp hair over the vertex, crown and frontal/parietal areas in a relatively characteristic pattern that is a continuum (described by Hamilton Norwood scale). The loss of scalp hair in men is called MPHL and is known to be a process driven by the androgen, dihydrotestosterone (DHT), which can be inhibited and to some extent reversed by finasteride which inhibits the conversion of testosterone to DHT. Minoxidil can also delay or reverse MPHL.

### Female Pattern Hair Loss

In addition to the progression of MPHL, both males and females develop diffuse hair loss in the frontal/parietal scalp called “thinning,” which begins between 12 and 40 years of age. Collectively, MPHL and diffuse thinning in males and females is termed “androgenetic alopecia.” Perhaps more than males, females notice (and complain of) diffuse hair thinning progressively in middle age more than males, perhaps because diffuse alopecia is more noticeable and problematic for females because they do not suffer from MPHL and retain the frontal hairline. In females, thinning is known as “Female Pattern Hair Loss (FPHL)” and may be caused or exacerbated by androgens (Price, 2003, J. Investig. Dermatol. Symp. Proc. 8:24-27).

Mechanistically, FPHL is thought to share some features with MPHL in terms of progressive reduction in the duration of anagen and progressive follicular miniaturization, although recent studies have found a prolongation of kenogen. As with MPHL, thinning of the hair, especially on the top of head, in addition to affecting younger Individuals, can also occur in older individuals when amounts of testosterone and DHT in the body are decreasing. This can either be an extension of FPHL from the earlier years or even start in the latter decades of life (i.e. age-related hair thinning).

### Cicatricial Alopecia

Scarring alopecia, also known as cicatricial alopecia, includes primary cicatricial alopecia (PCA) and secondary cicatricial alopecia. Primary cicatricial alopecia describes a rare group of diverse hair disorders that cause permanent destruction and scarring of the hair follicle in otherwise healthy men and women of all ages (http://www.carfintl.org/faq.html; Price V H, 2006, “The medical treatment of cicatricial alopecia,” Semin Cutan Med Surg 25:56-9). In PCA, the hair follicle is the primary target of a folliculocentric inflammatory attack that results in destruction and replacement of the sebaceous gland and follicular stem cells with fibrous (scar) tissue. Secondary cicatricial alopecia describes an incidental destruction of the follicular unit following severe infections, tumors, burns, or radiation.

Primary cicatricial alopecia represents at least eight rare diseases that cause permanent hair loss. The clinical course of these diseases is highly variable and unpredictable. Hair loss may slowly progress over many years, or may occur rapidly within months. Itching, pain and burning are often severe and incapacitating. Primary cicatricial alopecia is currently classified by the histopathological analysis of scalp biopsies, which stratifies those with a predomIn antly lymphocytic inflammation from those with a predomIn antly neutrophilic inflammation, and from those with a mixed infiltrate.

Lymphocyte-mediated PCA includes lichen planopilaris (LPP), frontal fibrosing alopecia (FFA), central centrifugal cicatricial alopecia (CCCA), and pseudopelade (Brocq).

Neutrophil-mediated PCA includes folliculitis decalvans and tufted folliculitis. A mixed inflammatory infiltrate occurs in dissecting cellulitis and folliculitis keloidalis, both of which are secondary to follicular rupture.

### Aging

Aging of humans results in programmed hair patterning. Diffuse hair loss, including thinning of the occipital scalp occurs in aging. This can either be an extension of androgenetic alopecia (MPHL or FPHL) from the earlier years or even start in the latter decades of life when amounts of testosterone and DHT in the body are decreasing.

It is believed that hair loss in postmenopausal women is related to the loss of estrogens (and/or a decrease in the estrogen/androgen ratio). Accordingly, in some embodiments, the combination treatments disclosed herein for age-related hair loss comprise a combination of treatment with one or more hair growth-promoting agents and estrogen replacement therapy or androgen inhibition therapy.

Aging also results in change of follicle cycle control. In males, eyebrows grow longer and nares hair grow longer suggesting that the lengths of telogen and anagen are no longer regulated as closely. In other words, with aging there is a loss of the function of suppressing terminal hair growth.

It may also be possible to predict who will respond better to Minoxidil by checking levels of minoxidil sulfotransferase (Buhl et al., J Invest Dermatol. 1990; 95(5):553-557). Minoxidil is converted to its active form, minoxidil sulfate, in the outer root sheath of the hair follicle by endogenous sulfotransferase enzymes. Three studies have reported a correlation between sulfotransferase activity in plucked hair follicles and minoxidil response for AGA patients (Goren et al., Novel enzymatic assay predicts minoxidil response in the treatment of androgenetic alopecia. Dermatol Ther 2013: 27 (3): 171-173; Roberts et al., Sulfotransferase activity in plucked hair follicles predicts response to topical minoxidil in the treatment of female androgenetic alopecia. Dermatol Ther 2014: 27 (4): 252-254; Goren et al., Clinical utility and validity of minoxidil response testing in androgenetic alopecia. Dermatol Ther. 2015 January-February; 28(1):13-6).All three studies show similar accuracies of 95.8%, 95.3%, and 100%, respectively, for ruling out nonresponders. Combined data from both studies demonstrates the diagnostic is 94% sensitive and 76% specific for predicting response to minoxidil treatment for AGA. These studies demonstrate the clinical utility and validity of minoxidil response testing in AGA patients and potentially patients with other hair loss disorders. Accordingly, in some embodiments, activity of endogenous sulfotransferase is measured prior to commencement of the methods described herein. Suitable techniques are described in e.g., Goren et al, 2015.

### Minoxidil Serum Levels

Topical minoxidil solution was originally developed as a treatment for AGA after the discovery that the active ingredient (minoxidil) caused hypertrichosis (excessive growth of hair) when taken orally for the treatment of hypertension ((Headington JT. 1987. Hair follicle biology and topical minoxidil: possible mechanisms of action; Dermatologica 175(Suppl 2):19-22).

The potential for cardiovascular events due to systemic effects of minoxidil was considered by FDA in its approval of minoxidil for OTC use. Serum minoxidil levels at which significant increases in heart rate were found begin at concentrations greater than 20 ng/mL, i.e., side effects were observed at serum concentrations of 21.7 ng/mL (Ferry JJ, et al. 1996. Hemodynamic Effects of Minoxidil Following Intravenous Infusions in Untreated Hypertensive Patients. Clinical Pharmacology and Therapeutics 59(2):166). In clinical trials of 2% and 5% minoxidil routine topical application, serum levels were evaluated and measurements demonstrated average serum levels of 0.7 and 1.7 ng/mL for 2% and 5% solution, respectively (U.S. Food and Drug Administration. 1997. Review Memoranda for NDA 20-834 (Pharmacia & Upjohn Company). Although these levels are well below the 20 ng/mL level where hemodynamic changes are observed, certain individuals experienced higher levels closer to the 20 ng/mL threshold, with a maximum serum recorded for the 5% solution of 16.5 ng/mL (Olsen et al. (2002)); A randomized clinical trial of 5% topical minoxidil versus 2% topical minoxidil and placebo in the treatment of androgenetic alopecia in men; J Am Acad Dermatol 2002; 47:377-85). However, since their approval by the FDA, topical minoxidil solution 2% and 5% have shown a long history of safe over the counter use. Adverse effects of topical minoxidil application are typically variations of local irritation (e.g., erythema, contact dermatitis), primarily thought to result from the vehicle (propylene glycol).

In a study using the methods, devices, and treatment regimens e.g., comprising integumental perturbation and post-perturbation minoxidil application, as described further in more detail herein and exemplified in Example 1, mean serum levels of 1.1 ng/mL were observed as representative of the peak concentration of serum minoxidil at 1 hour after treatment with 2% MTS. Accordingly, the mean observed serum levels were (a) higher than serum levels observed with 2% MTS (0.7 ng/ml), and (b) also remained below the levels observed with 5% MTS (1.7 ng/ml), which as noted above have been demonstrated to be safe clinically and in broad over-the-counter usage. Accordingly, in some embodiments, the methods, devices, kits, systems and treatment regimens provided herein provide a treatment which provides efficacy benefits over topical administration alone (described and exemplified in detail elsewhere herein) and keeps serum levels of minoxidil within ranges that have routinely been considered safe clinically and over the counter.

Accordingly, in certain embodiments, application to a subject of the methods, kits, systems, and treatment regimens disclosed herein results in a mean peak serum concentration of minoxidil in the subject of at most 0.5, 0.6, 0.7, 0.8, 0.9, 1.0, 1.1, 1.2, 1.3, 1.4, 1.5, 1.6, 1.7, 1.8, 1.9, or at most 2.0 ng/mL. In a specific embodiment, the mean serum concentration is 1.1 ng/ml.

Additionally, the maximum serum level recorded in the study described herein in Example 1 was 5.5 ng/mL. This is substantially lower than the MTS 5% maximum level of 16.5 ng/mL in an individual observed in the above described study assessing the efficacy of 5% topical minoxidil (Olsen et al. (2002); A randomized clinical trial of 5% topical minoxidil versus 2% topical minoxidil and placebo in the treatment of androgenetic alopecia in men; J Am Acad Dermatol 2002; 47:377-85). Accordingly, the methods, kits, systems, and treatment regimens described herein are employed to ensure that that the serum minoxidil levels remain within the levels generally accepted as safe. In some embodiments, the methods described herein comprising the steps of integumental perturbation and post-perturbation minoxidil application comprise a step up in concentrations of minoxidil applied (i.e., starting at 2% and step up to 5% minoxidil), allowing serum minoxidil levels to stay within range of what has been previously demonstrated to be safe for over the counter minoxidil treatments. In some embodiments, the step-up methods and treatment regimens described herein (starting at 2% and step up to 5% minoxidil) allows serum levels to increase more gradually, reducing accumulation of minoxidil in serum.

Accordingly, in certain embodiments, application to a subject of the methods, kits, systems, and treatment regimens disclosed herein results in a maximum serum concentration of minoxidil in an individual subject of at most 0.5, 0.6, 0.7, 0.8, 0.9, 1.0, 1.1, 1.2, 1.3, 1.4, 1.5, 1.6, 1.7, 1.8, 1.9, 2.0, 2.0-3.0, 3.0-4.0, 4.0-5.0, 5.0-6.0, 6.0-7.0, 7.0-8.0, 8.0-9.0, 9.0-10.0, 11.0 to 12.0, 12.0-13.0, 13.0 to 14.0, 14.0 to 15.0, 15.0 to 16.0 ng/mL. In a specific embodiment, the maximum serum concentration is 5 to 6 ng/ml.

In some embodiments, the methods described herein also provide a means to minimize topical side effects through employment of reduced levels of minoxidil and propylene glycol vehicle (which may be reduced in line with the minoxidil concentration). In some embodiments, local irritation (e.g., erythema, contact dermatitis) is reduced.

### Efficacy and Treatment Outcomes

Minoxidil topical solution has shown some efficacy across studies for the promotion of hair growth, for example, in one minoxidil study, both MTS 5% and minoxidil foam 5% achieved an average of 12% increase above baseline (non-vellus target area hair count) (Olsen 2002 and Olsen 2007). However, it is not effective in all individuals. For example, in one study of 2% MTS, 4 months of treatment yielded only 26% of individuals reporting moderate (defined as new individual hairs that covered all or some of the thinning areas but not as close together as hairs on the rest of the head) to dense hair regrowth (new hairs that cover or almost completely cover the thinning area and are as close together as hairs on the non-thinning areas of the head), as compared to 11% of subjects using a vehicle-only control. Using the methods, devices, kits, systems, and treatment regimens described herein, the number of respondents to the treatment can be significantly increased. In a study using the methods, devices, and treatment regimens e.g., comprising integumental perturbation according to the protocols described herein and post-perturbation minoxidil application, as described further in more detail herein and exemplified in Example 1, 79% of subjects showed an increase in non-vellus target area hair count greater than the above mentioned prior large minoxidil studies described in Olsen 2002 and Olsen 2007. Accordingly, this treatment protocol achieved positive efficacy results relative to minoxidil-only treatments. Using the methods described herein, a greater level of increase in non-vellus hair was observed as compared to minoxidil alone studies in the majority of subjects. Accordingly, in some embodiments, the methods devices, kits, systems, and treatment regimens described herein achieve increased hair growth to a greater extent than treatments with minoxidil alone in the majority of subjects. The greatest increase was observed with a regimen on biweekly perturbation, as described in Example 1. In some embodiments, integumental disruption is performed bi-weekly.

In some embodiments, application to a subject of the methods, kits, systems, and treatment regimens disclosed herein increases non-vellus hair count by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases vellus hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases terminal hair by 5% or more, by 10% or more, by 15% or more, by 20% or more, by 25% or more, by 30% or more, by 40% or more, by 50% or more, by 75% or more, or by 100% or more. In some embodiments, an a treatment regimen described herein increases total hair count by 5% or more, by 10% or more, by 15% or more, by 20% or more, by 25% or more, by 30% or more, by 40% or more, by 50% or more, by 75% or more, or by 100% or more.

In certain embodiments, such an improvement in total hair count, non-vellus hair, vellus hair, terminal hair, can be observed within or after 2 weeks, 1 month, 2 months, 3 months, 4 months, 5 months, 6 months, 7 months, 8 months, 9 months, 10 months, 11 months, or one year or longer after initiation of the treatment regimen. In certain embodiments, total hair count, non-vellus hair, vellus hair, terminal hair, can be observed using a macrophotography procedure as described herein. In a specific embodiment, detectable hairs can be quantified by photography, e.g., by global photographic recording or phototrichographic analysis (as described in, e.g., Uno et al, 2002, Acta Venereol 82:7-12, incorporated herein by reference). Further, changes in the hair shaft thickness of photographically detectable hairs can be determined. In certain embodiments, the permanence of the hair growth is monitored over a time period of at least 2 months, 3 months, 4 months, 5 months, 6 months, 7 months, 8 months, 9 months, 10 months, 1 1 months, 1 year, 1.5 years, 2 years, 2.5 years, 3 years, 4 years, or at least 5 years or more.

Any method known to the skilled artisan can be used to demonstrate success of a treatment described herein. In various embodiments, success of treatment aimed at treating alopecia, treating baldness, or promoting hair growth can be measured according to one or more of the following methods: improved overall cosmetic outcome (e.g., using the Visual Analogue Scale (VAS)); patient assessment of his/her hair growth (e.g., based on questionnaire); investigator assessment of hair growth in a patient (e.g., based on a rating scale); patient assessment of his/her hair growth in photographs; investigator assessments of hair growth in patient photographs; increased hair count (e.g., by measuring new hair growth as an increased number of fibers in an affected area of the skin); increased hair density; increased thickness of hair or hair shaft (e.g., based on diameter); increased hair weight; hair cuttings; longer hair; an increase in the number of photographically detected hairs; increase in the amount of terminal hair (by, e.g., measuring new hair growth as an increased number of fibers in an affected area of the skin, or increased thickness (e.g., diameter) or length of hair fibers (e.g., as measured photographically)); increase in the amount of vellus hair (by, e.g. , measuring new hair growth as an increased number of fibers in an affected area of the skin) (e.g. , as measured photographically); increase in the amount of non-vellus hair, e.g. , intermediate or terminal hair (e.g., as measured photographically); an increase in the ratio of terminal-to-vellus hair; increased number of hair germs; increased number of hair follicles (e.g., as evaluated by a skin biopsy); increased number of hair follicles at a more mature stage of development; increased numbers of follicular units with 3 or more hair follicles; increased hair follicle branching; formation of new hair follicles (“hair follicle neogenesis”); formation of new hair follicles with vellus-sized hair shafts (i.e., hair shafts with diameters less than 30 microns in diameter)

formation of new hair follicles with non-vellus-sized hair shafts (i.e., hair shafts with diameters 30 microns or greater in diameter); hair follicle regeneration; increased activation of existing hair follicles; increased number of hair follicles; increased number of activated or stimulated hair follicles; increased number of activated or stimulated pre-existing hair follicles; presence or increased numbers of neogenic-like (NL) hair follicles (based on, e.g., examination of a biopsy or by confocal microscope, by assessing number of hair follicles, and/or by assessing morphology of hair follicles compared to baseline or a negative control); presence or increased numbers of pre-existing hair follicles (based on, e.g., examination of a biopsy or by confocal microscope, by assessing number of hair follicles, and/or by assessing morphology of hair follicles compared to baseline or a negative control); presence or increased numbers of NL, PEL, and/or PELA follicular structures (based on, e.g., examination of a biopsy or by confocal microscope, by assessing number of hair follicles, and/or by assessing morphology of hair follicles compared to baseline or a negative control, as described for example in Sections 5.8.4.1 and 5.8.4.2); increased number of pre-existing hair follicles with vellus-sized hair shafts in a treated area of skin of a subject; increased number of neogenic-like hair follicles with vellus-sized hair shafts in a treated area of skin of a subject; increase in the amount of anagen hair; increase in the amount of telogen hair; increased proportion of hair follicles in anagen or decreased proportion of hair follicles in telogen (e.g., an increase in the ratio of anagen-to-telogen hair) (e.g. , based on examination of a biopsy or phototrichograms); increased proliferation of dermal papilla (based on, e.g. , examination of a biopsy); increased recruitment or proliferation of stem cells to the follicle (e.g. , based on examination of a biopsy).

For example, in certain embodiments, success of treatment is assessed by determination of the percentage increase in total hair. In some embodiments, increase in vellus and non-vellus hair is assessed. In some embodiments, increase in hair diameter is assessed. Without wishing to be bound by theory, increase in hair diameter may indicate transition from vellus to non-vellus hair. In some embodiments, hair mass is assessed.

In a particular embodiment, measurement of total hair counts, vellus hair counts, non-vellus hair counts, hair mass, and/or number of thicker hairs in accordance with the foregoing provides a means for evaluating success of a method of integumental perturbation in combination with post-perturbation minoxidil treatment, as described herein. In an exemplary, non-limiting embodiment, success of a method of integumental perturbation in combination with post-perturbation minoxidil is determined based on a measured increase in total hair counts, vellus hair counts, non-vellus hair counts, hair mass, and/or increases in number of thicker hairs in an area of skin subjected to integumental perturbation and post perturbation minoxidil, for example, compared to an area of skin that was not subjected to the integumental perturbation step, i.e., was either untreated or treated with minoxidil alone.

In certain embodiments, success of treatment is assessed by measuring hair count in a treated area of skin. For example, detectable hairs can be quantified by photography, e.g., by global photographic recording or phototrichographic analysis (as described in, e.g., Uno et al, 2002, Acta Venereol 82:7-12, incorporated herein by reference). Further, changes in the hair shaft thickness of photographically detectable hairs can be determined. In certain embodiments, the permanence of the hair growth is monitored over a time period of at least 2 months, 3 months, 4 months, 5 months, 6 months, 7 months, 8 months, 9 months, 10 months, 1 1 months, 1 year, 1.5 years, 2 years, 2.5 years, 3 years, 4 years, or at least 5 years or more.

In some embodiments, a treatment regimen described herein increases total hair count by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases vellus hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases terminal hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein results in 1-5%, 5-10%, 10-15%, 15-20%, 20-25%, 25-30%, 30-35%, 35-40%, 40-45%, 45-50%, 50-75%, or 75-100% conversion of vellus hair to non-vellus (i.e., intermediary or terminal hair). In some embodiments, a treatment regimen described herein increases hair thickness by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases hair shaft diameter by approximately 1, 2, 2.5, 3, 3.5, 4, 4.5, 5, 5.5, 6, 6.5, 7, 7.5, 8, 8.5, 9, 10, 15, 20, 25, or 30 microns or more. In some embodiments, a treatment regimen described herein increases mean hair shaft diameter by 5% or more, by 10% or more, by 15% or more, by 20% or more, by 25% or more, by 30% or more, by 40% or more, by 50% or more, by 75% or more, or by 100% or more. In some embodiments, a treatment regimen described herein results in 1-5%, 5-10%, 10-15%, 15-20%, 20-25%, 25-30%, 30-35%, 35-40%, 40-45%, 45-50%, 50-75%, or 75-100% increase in mean hair shaft diameter. In some embodiments, a treatment regimen described herein increases the ratio of terminal to vellus hair follicles by 5% or more, by 10% or more, by 15% or more, by 20% or more, by 25% or more, by 30% or more, by 40% or more, by 50% or more, by 75% or more, or by 100% or more. Such an improvement in hair count, vellus hair, terminal hair, conversion of vellus hair to non-vellus (e.g., intermediate or terminal) hair, hair thickness, hair shaft diameter, or the ratio of terminal to vellus hair may be measured within or after 1 month, 2 months, 3 months, 4 months, 5 months, 6 months, 7 months, 8 months, 9 months, 10 months, 11 months, or one year or longer after initiation of the treatment regimen.

In certain embodiments, improvement in hair count, vellus hair, terminal hair, conversion of vellus hair to non-vellus (e.g., intermediate or terminal) hair, hair thickness, hair shaft diameter, or the ratio of terminal to vellus hair may be measured within 3 days, or 4 days, 5 days, 6 days, 7 days, 8 days, 9 days, 10 days, 11 days, 12 days, 13 days, 14 days, 15 days, 16 days, 17 days, 18 days, 3 weeks, 4 weeks, or 1 month or longer after initiation of the treatment regimen described herein.

In a particular embodiment, improvement in hair count, vellus hair, terminal hair, conversion of vellus hair to non-vellus (e.g., intermediate or terminal) hair, hair thickness, hair shaft diameter, or the ratio of terminal to vellus hair may be measured at 1, 15, 29, 43, 57, and 71 days after initiation of the treatment regimen described herein. In a particular embodiment, improvement in hair count, vellus hair, terminal hair, conversion of vellus hair to non-vellus (e.g., intermediate or terminal) hair, hair thickness, hair shaft diameter, or the ratio of terminal to vellus hair may be measured at 1, 29, and 57 days after initiation of the treatment regimen described herein. In a particular embodiment, measurement of total hair counts, vellus hair counts, non-vellus hair counts, hair mass, and/or number of thicker hairs in accordance with the foregoing is based on measurements taken 11, 29, and 57 days after initiation of the treatment regimen described herein. In a particular embodiment, measurement of increases in total hair counts, vellus hair counts, non-vellus hair counts, hair mass, and/or increases in number of thicker hairs in accordance with the foregoing is at 1, 8, 15, 22, 29, 36, 43, 50, 57, 64, 71, and 78 days after initiation of the treatment regimen described herein. In a particular embodiment, measurement of total hair counts, vellus hair counts, non-vellus hair counts, hair mass, and/or number of thicker hairs in accordance with the foregoing is based on measurements taken 1, 8, 15, 22, 29, 36, 43, 50, 57, 64, 71, and 78 days after initiation of the treatment regimen described herein.

In some embodiments, a treatment regimen described herein increases hair count by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases vellus hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases terminal hair by 5% or more, by 10% or more, by 15% or more, by 20% or more, by 25% or more, by 30% or more, by 40% or more, by 50% or more, by 75% or more, or by 100% or more. In some embodiments, an a treatment regimen described herein results in 1-5%, 5-10%, 10-15%, 15-20%, 20-25%, 25-30%, 30-35%, 35- 40%, 40-45%, 45-50%, 50-75%, or 75-100% conversion of vellus hair to non-vellus (i.e., either intermediary or terminal hair). In some embodiments, an a treatment regimen described herein increases hair thickness by 5% or more, by 10% or more, by 15% or more, by 20% or more, by 25% or more, by 30% or more, by 40% or more, by 50% or more, by 75% or more, or by 100% or more. In some embodiments, an a treatment regimen described herein increases hair shaft diameter by approximately 1, 2, 2.5, 3, 3.5, 4, 4.5, 5, 5.5, 6, 6.5, 7, 7.5, 8, 8.5, 9, 10, 15, 20, 25, or 30 microns or more. In some embodiments, a treatment regimen described herein increases hair shaft diameter by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases the ratio of terminal to vellus hair follicles by 5% or more, by 10% or more, by 15% or more, by 20% or more, by 25% or more, by 30% or more, by 40% or more, by 50% or more, by 75% or more, or by 100% or more. Such an improvement in hair count, vellus hair, terminal hair, conversion of vellus hair to non-vellus (i.e., either intermediate or terminal) hair, hair thickness, hair shaft diameter, or the ratio of terminal to vellus hair may be measured within or after 1 month, 2 months, 3 months, 4 months, 5 months, 6 months, 7 months, 8 months, 9 months, 10 months, 11 months, or one year or longer after initiation of the treatment regimen.

In certain of the foregoing embodiments, the increase in hair count, vellus hair, terminal hair, conversion of vellus hair to non-vellus (i.e., either intermediate or terminal) hair, hair thickness, hair shaft diameter, and/or the ratio of terminal to vellus hair is observed in the treated area, for example, in an area of skin that was treated with integumental perturbation. In other embodiments, the increase in hair count, vellus hair, terminal hair, conversion of vellus hair to non-vellus (i.e., intermediate or terminal) hair, hair thickness, hair shaft diameter, and/or the ratio of terminal to vellus hair is observed adjacent to the treated area. In other embodiments, the increase in hair count, vellus hair, terminal hair, conversion of vellus hair to non-vellus (e.g., intermediate or terminal) hair, hair thickness, hair shaft diameter, and/or the ratio of terminal to vellus hair is observed in and adjacent to the treated area.

In a particular embodiment, measurement of hair in accordance with the foregoing provides a means for evaluating whether a subject is a candidate for treatment, or continued treatment, with the methods described herein. In one embodiment, a method of treatment is carried out over a small area of skin (e.g., 1×1 cm, or 1.5×1.5 cm, or 2×2 cm, or 2.5×2.5 cm, or 3×3 cm or more), hair is measured in accordance with a foregoing methods, and if candidacy is established, the method of treatment is carried out over a larger area of skin, such as, e.g., an entire balding area of scalp.

### Needling Devices

In some embodiments, the needling device is a powered needling device which may, in some embodiments, be configured to drive a plurality of needles in a reciprocating motion to produce small wounds in skin. The needling device may drive the needles to oscillate up-and-down rapidly in a vertical motion, as the user passes the needling device across the treatment area.

Details regarding different embodiments of a needling device are described in U.S. Published Application No. 2018/0280675, which is hereby incorporated by reference in its entirety.

In some embodiments, the needling device consists of a reusable electromechanical core enclosed in a single-use disposable sterile sheath. The reusable core may contain a power source, such as a rechargeable lithium-ion battery, a motor to actuate the needling component, and user controls. The user controls may allow a user to, for example, turn on and turn off the needling device, adjust the needle drive speed, adjust illumination at the needle-end of the sheath, and/or perform other suitable functions. The core may be sealed and fully wipeable, designed for re-use and can undergo disinfection.

The disposable sheath may be single-use, designed to prevent fluid ingress into the reusable core, and is provided sterile to reduce risk of infection from the procedure. The needle array component is integrated into the sterile, single-use sheath. In some embodiments, the needle array comprises 12 needles.

The needling device may be packaged in a kit containing a plurality of components. In some embodiments, a kit may include a device core, a core charger, and instructions for use. The kit may also include a plurality of sheaths, each sheath being individually packaged and sterilized.

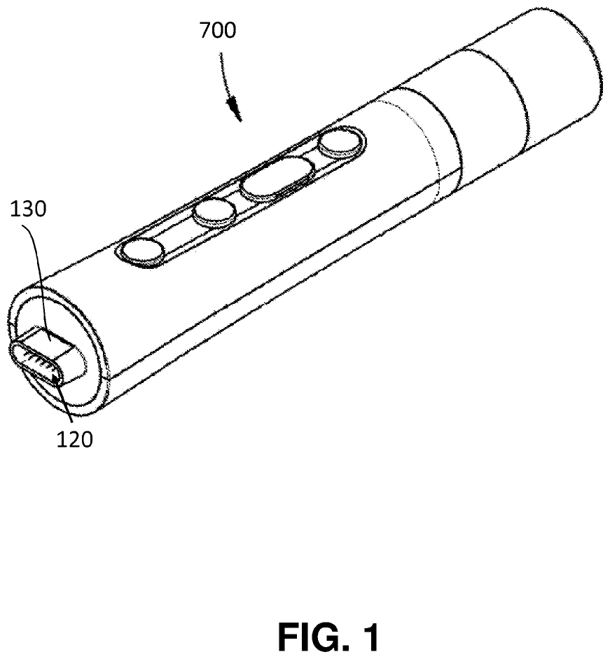

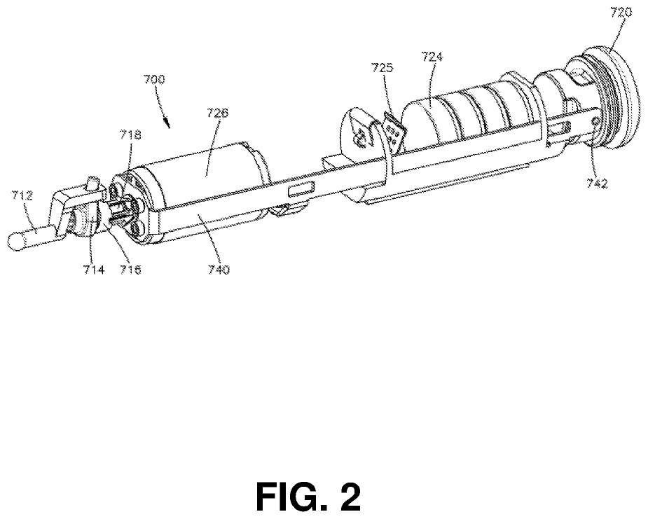

One illustrative embodiment of a needling device is shown in FIGS. 1 and 2, where FIG. 1 is a perspective view of a first embodiment of a needling device 700, and FIG. 2 is a is a perspective view of the inner components of the needling device of FIG. 1. Referring to FIG. 1, the needling device includes an array of needles 120, and a head covering 130 surrounding the needles. According to one aspect, the head covering 130 may serve as a guiding feature that aids a user in determining how much pressure to apply the needling device against the skin. As will be discussed below, in some embodiments, application of too much or too little pressure may affect treatment performance.

Referring to FIG. 2, the inner components of the needling device 700 include a micropen cap 720, a battery 724, a battery clip 725, and a motor 726. In addition, the needling device 700 includes a motor shaft 718 that extends from the motor 726, an angled hub 716 secured to the motor shaft 718, a wobble plate 714 that is received by the angled hub 716, and a drive shaft 712 that is connected to the wobble plate 714. The inner components of the needling device 700, including at least the motor 726, the motor shaft 718, the angled hub 716, the wobble plate 714, and the drive shaft 712, are connected by a chassis 740 that is configured to hold together all components and move the components along the longitudinal axis of the needling device 700. The chassis 740 may be attached to the micropen cap 720 by a chassis pin 742 so that rotation of the micropen cap 720 in one direction moves the inner components axially in one direction towards or away from the subject's skin, and rotation of the micropen cap 720 in the opposite direction moves the inner components in the opposite direction. This in turn controls the penetration depth of the needles.

It is important to distinguish the operation of a powered, vertically reciprocating microneedling device, e.g., as described above and in U.S. Published Application No. 2018/0280675, from different class of device known as microneedle rollers. Microneedle rollers utilize similarly sized, small gauge needles to disrupt the skin, however, both the a) device operation and b) resulting nature of skin disruption are significantly differ from device operation and skin disruption achieved using powered microneedling devices. The primary source of difference is that the action of each needle in a microneedle roller causes a small cut or scrape, resulting from a sweeping motion of the needle relative to the surface of the skin as the drum revolves; in contrast, each needle in a powered microneedling device creates a narrow column of disruption from a vertical insertion and extraction motion. The degree and nature of disruption per needle strike is significantly different as a result of this difference.

The primary reason that powered microneedling devices do not create a cut or scraping effect is due to their very fast oscillation rate relative to the speed of translation of the device across the skin. As a result of this relative difference, the needle is inserted into the skin for a very short duration, after which the minimal dragging tension from translation of the device is released before it can create additional disruption (i.e., a scraping effect). The vertically oscillating needle then lifts out of the skin for a duration of time, before puncturing at the next location. For example, the device described herein and employed in the methods and treatment regimens in the Examples herein is set at 120 Hz oscillation frequency, and, translating at 2 cm/s along the skin, travels ˜170 μm along the skin between each successive needle strike. However, the needle is only in contact with the skin for ˜25% of the stroke duration (˜2.0 ms out of 8.3 ms stroke duration) as the needle protrudes from the device only for the final 0.8 mm of its full 3.0 mm extension per cycle. Ultimately, this means the needle travels only ˜40 μm in the direction of device translation while in contact with the skin. This short travel distance ensures each needle strike is creating a narrow column of disruption, perpendicular to the surface of the skin. This type of disruption not only better maintains the integrity of the skin at the surface layer, but also achieves more even tissue disruption along the affected column.

Microneedle rollers, in contrast, consist of many rows of needles affixed radially to a rotating “drum” that is applied with pressure and rolled across the skin. As the drum rolls, successive rows of needles encounter, penetrate, and then exit the skin. Picturing a single row of needles that is first encountering the skin layer, the needles are not pointed vertically, but instead are at an angle pointed toward the direction of motion. The rolling action of the drum and applied pressure cause that row of needles to pierce the skin. As the drum continues to roll, the needle angle (measured away from vertical) decreases and the penetration depth increases. That row of needles will penetrate to its deepest point generally when the needles are pointed directly downwards (vertical). The row then rotates to angle away from the direction of motion, ultimately exiting the skin once it reaches a similar angle away from vertical. This type of sweeping travel creates a scraping effect on the skin. The damage to the skin layer is greatest at upper layers of the skin, because the needle has not achieved its full depth when it first encounters the skin and begins its travel.

For a roller with similar length needle to the device needle extension example (e.g., 0.8 mm) and a typical roller drum radius (e.g., 10 mm), each needle tip travels ˜8 mm relative to the skin from puncture to removal as the drum rotates, causing the scraping effect. By comparison, this distance is ˜200× greater than the distance a powered microneedler needle interacts with the skin per strike (˜40 μm noted above). The more localized puncture effect of a powered microneedling device needle strike enables these devices to achieve a much greater needle strike density than rollers, for comparable degrees of clinical endpoint (erythema, pinpoint bleeding). For example, calculations by the inventors show a similar order of magnitude of difference (˜100×) in needle strike density between a powered microneedler device and a roller treatment, where both devices are titrated to achieve similar grades of erythema (skin redness) and pinpoint bleeding.

In some embodiments, the device as described herein is set at 120 Hz oscillation frequency. In some embodiments, the device translates at 2 cm/s along the skin, traveling ˜170 μm along the skin between each successive needle strike. In some embodiments, the device travels ˜170 μm along the skin between each successive needle strike. In some embodiments, the needle is contact with the skin for ˜25% of the stroke duration. In one non-limiting example, the needle is in contact with the skin approximately 2.0 ms out of a 8.3 ms stroke duration. In some embodiments, the needle travels ˜40 μm in the direction of device translation while in contact with the skin.

### Device Use Procedure

One illustrative embodiment of a treatment method will now be described. The subject is asked to remain still and relaxed while in a reclined but sufficiently upright position to treat the target area. The target treatment area includes the vertex and the transitional areas which should include at least 2 cm from the leading edge of the actively balding scalp area. The needle depth for the procedure is set at 0.8 mm, and the frequency of needle reciprocation is set at 120 Hz. Treatment density is standardized with each area of the scalp receiving two passes of the needling device, with movement speed of the needling device across the scalp targeted to be 2 cm/s. With this treatment method, each treatment is anticipated to last approximately five minutes or less.

If the procedure needs to be paused, the device can be removed from contact with the subject's scalp and powered off. The practitioner administering the treatment should keep track of any area not yet treated. Treatment can be resumed by powering the device back on. Following completion of the procedure, the sheath is discarded in an appropriate sharps container and the core is cleaned and inserted into the provided charger.

In some embodiments, skin is disrupted using the needling device, with a depth setting of 0.8 mm. The user glides the needling device over the affected skin area at a speed of 2 cm/second, maintaining the device perpendicular to the surface of the skin to promote a consistent needle puncture depth of 0.8 mm depth. The user glides the needling device across the affected area of the skin such that each pass across the area of skin is contiguous to the prior pass across the skin, minimizing gaps between and overlap of the passes, in a “mow the lawn” approach. Each pass of the needling device starts with a gliding start and a smooth retraction of the device from the skin area following each pass. The needling device is applied to the skin area with consistent, light pressure, allowing the device to glide along the skin.

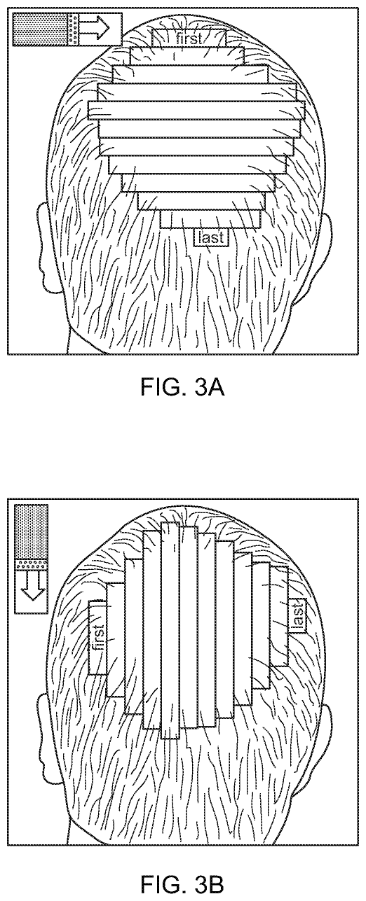

One illustrative embodiment of the “mow the lawn” approach is shown in FIGS. 3A and 3B, where each elongated rectangle represents one pass of the device across the skin. As can be seen from the images, the passes are adjacent to one another to avoid gaps between and overlap of contiguous passes. FIG. 3A shows horizontal passes across the skin, while FIG. 3B shows vertical passes along the skin. In some embodiments, more than one series of passes is applied to a treatment area. For example, a first series of passes, may be applied to a treatment area. Then, a second series of passes, which may have a different direction than that of the first series of passes, may be applied to the treatment area. In some embodiments, the second series of passes have a direction perpendicular to the first series of passes.

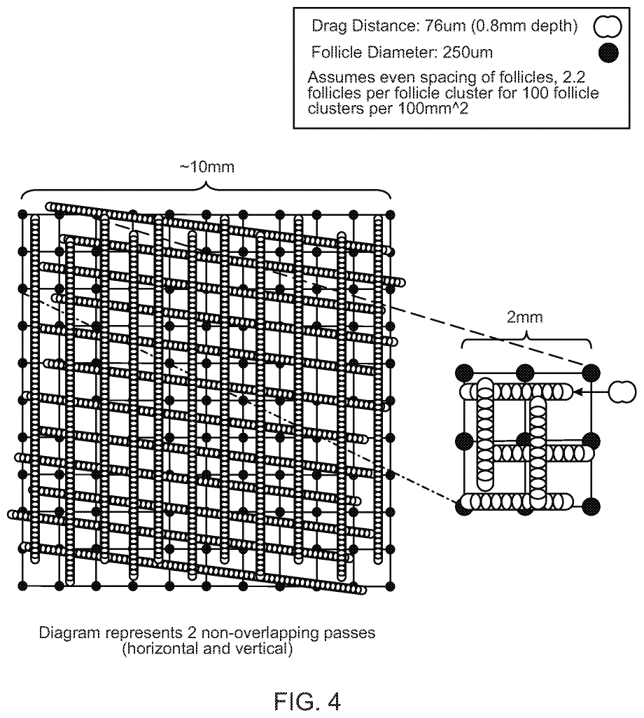

As an illustrative embodiment, the horizontal passes of FIG. 3A are the first series of passes applied to a treatment area, and the vertical passes of FIG. 3B are the second series of passes applied to the treatment area. A diagram of the resulting needle strikes using horizontal and vertical passes is shown in FIG. 4.

In one illustrative embodiment, the needling device has a device alignment consisting of 6 rows of 2 needles. The device array has 12 needles and the number of array oscillations per second is set at 120. The array width is 0.9 cm, and the translation speed is about 2.0 cm/s. Two treatment passes are performed, one horizontal and the other vertical such that the treatment passes are perpendicular to one another. As a result, 1600 needle strikes per cm\hat{ }2 result.

### Device Treatment Parameters

The inventors have identified several treatment parameters that may affect treatment results.

In some embodiments, one parameter is the speed of device translation, which is the speed that the user moves the device (i.e., in linear passes) determines density of needle strikes, which is a key parameter defining the intensity of treatment. In one embodiment, the device translation is at 2 cm/s. Given a frequency of needle array reciprocation (e.g., 120 Hz), this creates the desired density of needle strikes with each strike slightly overlapping the prior. In some embodiments, a ruled 20 cm guideline grid, with tick-marks every 2 cm, drawn on top of vinyl-lined foam can be used as a tool for monitoring the speed of the device. An audible metronome may be used to keep the pace (e.g., 60 Hz), and the user steadily translates the device to cross 1 tick mark per metronome beep. The user's speed may be monitored in multiple training session using a timer and, in some cases, a video camera. This may enable training with an active device to ensure proper speed is met.

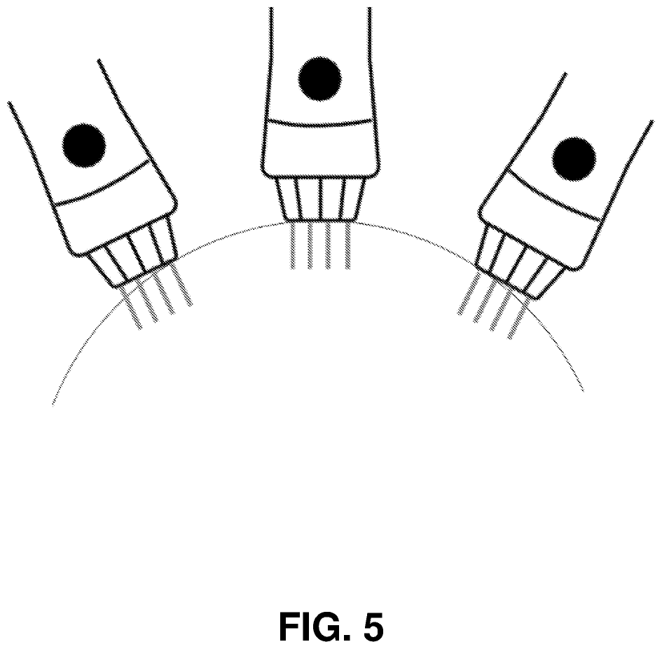

In some embodiments, another parameter is the perpendicularity of the needling device. The device should be kept normal to the skin in both the lateral (side to side) and translational (line the user is moving the device) directions. This ensures that the face of needle array will be at an even distance from the skin, and the needle depth will not have undesired variability across the array. If the orientation of the device relative to the skin is not maintained at a perpendicular angle, some of the needles will not achieve their full intended insertion depths. For example, as illustrated in FIG. 5, the needling devices at the left and right sides of the figure are not perpendicular to the skin, and thus some of the needles are not at their full insertion depths. In contrast, the needling device shown in the middle of the figure is perpendicular to the skin, and all its needles are at their full insertion depths.

Training methods employ the use of the flat surface (foam+vinyl) grid described above. Additional training methods include the use of a human head model with a foam/vinyl “yarmulke” that models the vertex area of treatment.

In some embodiments, another parameter is device pass contiguity and orientation: it is important that each pass is contiguous to the prior (“mowing the lawn”), without overlap or gaps. This ensures no missed areas or areas of significantly greater density due to overlap. As discussed previously, illustrative examples of “mowing the lawn” are shown in FIGS. 3A and 3B.

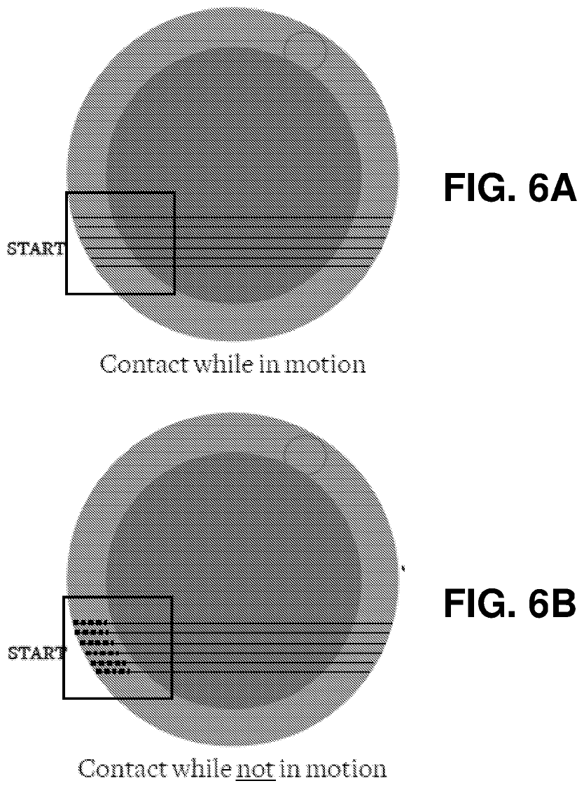

In some embodiments, another parameter is pass initiation: initiation of a treatment pass is at a moving start, so that there is not unintentionally higher density at the edge of the treatment area. FIGS. 6A and 6B illustrate this concept. In FIG. 6A, the user has used a moving start, meaning that as the user places the device against the skin, the user simultaneously translates the device across the skin without holding the device at the initial contact point for a prolonged period of time. As a result, a more uniform needle strike density is achieved. In contrast, in FIG. 6B, when the user first places the device against the skin, the user initially holds the device at the starting point prior to translating the device across the skin, resulting in a higher density of needle strikes at the starting point.

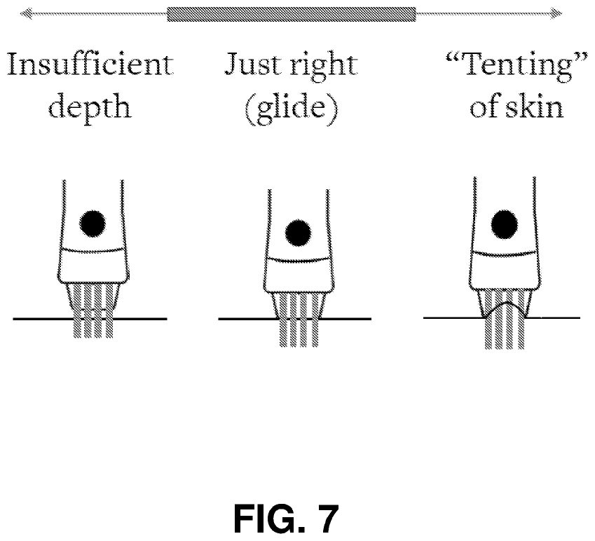

In some embodiments, another parameter is pressure of contact: the user achieves only a light gliding pressure that ensures the device is against skin but does not apply undue pressure such that the skin “tents” into the device and the achieved needle depth is greater than preferred. This concept is illustrated in FIG. 7. On the left side of the figure, the user has applied insufficient pressure against the skin, resulting in insufficient penetration depth of the needles. On the right side of the figure, the user has applied too much pressure against the skin, resulting in “tenting” of the skin in which skin has bunched up and needle penetration depth is too high. According to this aspect, the user seeks to achieve just the right amount of pressure against the skin using a light gliding pressure, illustrated in the middle of the figure.

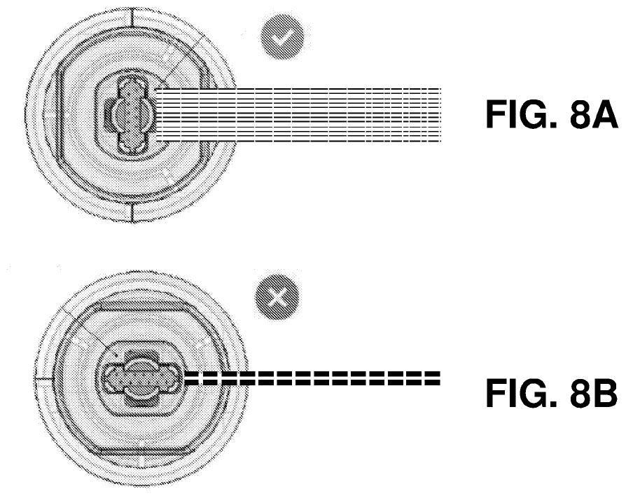

In some embodiments, another parameter is the angular orientation of the needling device. The user may seek to ensure that the lines of needles in the array extend in a direction that is perpendicular to the direction of translation of the device. In some embodiments, moving at an angle that is not perpendicular to the extension direction of the lines of needles could result in “stripes” of greater and lesser needle strike densities. An illustration of this concept is shown in FIGS. 8A and 8B. In the illustrative embodiments of FIGS. 8A and 8B, the needle array includes 2 lines of needles, each line having 6 needles. The 2 lines of needles are offset from one another. In FIG. 8A, the extension direction of the two lines of needles (vertical) is perpendicular to the translation direction of the device (horizontal), resulting in a more uniform needle strike density. In contrast, in FIG. 8B, the extension direction of the two lines of needles (horizontal) is parallel to the translation direction of the device (also horizontal), resulting in areas of greater needle strike density, as some needles at the left side of the array may strike areas of the skin that have already been pierced by needles on the right side of the array.

In some cases, especially for women, hair loss is more diffuse, such that treatment areas have moderate, often long, pre-existing hair which must be managed in order to properly treat the desired area.

In some embodiments, in the presence of longer hair, the device is moved along the scalp in a direction that is along the direction of the hair. Traveling against the hair could “catch” on perpendicular hairs, diminish the consistency of treatment, and could cause other unintended effects in existing long hair.

In one illustrative example of a treatment method with longer hair, existing hair is combed to make a linear part up and down the scalp in the most natural parting direction, and the section is treated with the needling device using short, contiguous, ˜2 cm strokes emanating from the center-line of the part. Each stroke is performed for one second to maintain even density. This process may be repeated twice in the same area to achieve desired two-pass density. After a section is treated (2 cm in either direction of the part), the next part is made 4 cm over, and the process is repeated as needed until the entire treatment area is covered.

### Needle Strike Density

With respect to achieving integumental perturbation using micro-needling, the needle strike density is an important property of the treatment, as it is a significant determinant of key treatment outcomes, especially treatment effect size, procedure tolerability, and procedure safety. Several key treatment parameters interact to define needle strike density; these can be grouped into those determining (1) number of needle strikes, (2) treatment area, and (3) number of passes. In general, the goal is to achieve needle strikes that provide a uniform density of evenly spaced needle strikes with minimal distance between the strikes over a specified area of skin. An optimal strike density can be achieved using various combinations of parameters selected from: the number of needles in the needle array, the width of the needle array, the number of array oscillations per second, the device translation speed, and the treatment area. For example, in some embodiments, the needle strike density is 1600 needle strikes per cm² or about 1600 needle strikes per cm².

First, the number of (1) needle strikes per second can be calculated by multiplying: (1A) number of needles per needle array by (1B) the number of array oscillations per second.

Second, the treatment area covered per second can be calculated by multiplying (2A) width of treatment (e.g., measured as the width of the needle array) by (2B) device translation speed. Overall, (1) the number of needle strikes, divided by (2) treatment area, multiplied by (3) the number of overlapping passes, results in the needle strike density. Several permutations of these treatment parameters exist in which equivalent treatment density is achieved.

In some embodiments of the methods described herein, one, two, three, four, or five passes are performed across the treatment area. In some embodiments, one pass is performed. In some embodiments, two passes are performed. In some embodiments, three passes are performed.

In some embodiments of the methods described herein, the needle array has 4, 8, 12, 16, or 20 needles. In some embodiments of the methods described herein, the needle array has 5, 10, 15, 20, or 25 needles. In some embodiments of the methods described herein, the needle array has 6, 12, 18, 24, or 30 needles. In some embodiments of the methods described herein, the needle array has 7, 14, 21, 28, or 35 needles. In some embodiments of the methods described herein, the needle array has 8, 16, 24, 32, or 40 needles. In some embodiments, the needle array has 12 needles. In some embodiments, the needle array has 16 needles. In some embodiments, the needle array has 24 needles.

In some embodiments of the methods described herein the number of array oscillations per second is at least about 80 to 90, 90 to 100, 100 to 110, 110 to 115, 115 to 120, 120 to 125, 125 to 130, 130 to 140, or 140 to 150. In one embodiment, the number of array oscillations per second is 120.

In some embodiments of the methods described herein, the width of treatment is at least about 0.4 to 0.5 cm, 0.5 to 0.6 cm, 0.6 to 0.7 cm, 0.7 to 0.8 cm, 0.8 to 0.9 cm, 0.9 to 1.0 cm, 1.0 to 1.1 cm, 1.1 to 1.2 cm, or 1.2 to 1.3 cm. In one embodiment, the width of treatment is at least about 0.9 cm. In one embodiment, the width of treatment is 0.9 cm.

In some embodiments of the methods described herein, the device translation speed is 1.5 to 2 cm/s, 2 to 2.5 cm/s, 2.5 to 3 cm/s, 3 to 3.5 cm/s, or 3.5 to 4 cm/s. In some embodiments of the methods described herein, the device translation speed is at least about 2 cm/s. In some embodiments of the methods described herein, the device translation speed is 2 cm/s.

In some embodiments of the methods described herein, the strike density is 900 to 1000, 1000 to 1100, 1100 to 1200, 1200 to 1300, 1300 to 1400, 1400 to 1500, 1500 to 1600, 1600 to 1700, 1700 to 1800, 1800 to 1900, or 1900 to 2000 needle strikes per cm\hat{ }2. In some embodiments of the methods described herein, the strike density is 900 to 2000, 1000 to 1900, 1100 to 1800, 1200 to 1800, 1400 to 1800, 1500 to 1700, 1550 to 1650, or 1575 to 1625 needle strikes per cm\hat{ }2. In some embodiments of the methods described herein, the strike density is 1550 to 1575, 1575 to 1600, 1600 to 1625, 1625 to 1650, or 1650 to 1700 needle strikes per cm². In some embodiments of the methods described herein, the strike density is 1600 needle strikes per cm².

For example, a treatment density of 1600 needle strikes per cm² can be achieved by different combinations of the described parameters.

In some embodiments of the methods described herein, the needle array has about 12 needles, and about 2 treatment passes are performed, wherein the number of array oscillations per second is set at about 120, the treatment width is about 0.9 cm, and the translation speed is about 2.0 cm/s. In some embodiments of the methods described herein, the needle array has 12 needles, and 2 treatment passes are performed, wherein the number of array oscillations per second is set at 120, the treatment width is 0.9 cm, and the translation speed is 2.0 cm/s. In some embodiments of the methods described herein, the needle array has about 24 needles, and about 1 treatment pass is performed, wherein the number of array oscillations per second is set at about 120, the treatment width is about 0.9 cm, and the translation speed is about 2.0 cm/s. In some embodiments of the methods described herein, the needle array has 24 needles, and 1 treatment pass is performed, wherein the number of array oscillations per second is set at 120, the treatment width is 0.9 cm, and the translation speed is 2.0 cm/s.

In some embodiments of the methods described herein, the needle array has about 12 needles, and about 1 treatment pass is performed, wherein the number of array oscillations per second is set at about 120, the treatment width is about 0.9 cm, and the translation speed is about 1 cm/s. In some embodiments of the methods described herein, the needle array has 12 needles, and 1 treatment pass is performed, wherein the number of array oscillations per second is set at 120, the treatment width is 0.9 cm, and the translation speed is 1 cm/s. In some embodiments of the methods described herein, the needle array has about 16 needles, and about 3 treatment passes are performed, wherein the number of array oscillations per second is set at about 90, the treatment width is about 1.8 cm, and the translation speed is about 1.5 cm/s. In some embodiments of the methods described herein, the needle array has 16 needles, and 3 treatment passes are performed, wherein the number of array oscillations per second is set at 90, the treatment width is 1.8 cm, and the translation speed is 1.5 cm/s.

### Needle Strike Depth

As used herein, integumental perturbation refers to any treatment of the skin and/or other tissues of the integumentary system that results in wounding, debriding, peeling, or other perturbation of the skin. In certain embodiments, a treatment with integumental perturbation refers to any treatment that results in an increase in the number of vellus hairs or terminal hairs or anagen hairs. integumental perturbation can be achieved by any means known in the art or described herein or that may become available in the future, such as, for example, using chemical, mechanical, physical, or electromagnetic means. In one embodiment, the integumental perturbation treatment increases the number of follicular structures in an area of skin adjacent to the integumentally perturbed skin site. In another embodiment, the integumental perturbation treatment increases the number of follicular structures in the integumentally perturbed skin site, for example, beneath the site of integumental perturbation. In another embodiment, the integumental perturbation treatment increases the number of follicular structures in the integumentally perturbed skin site and in an area of skin adjacent to the integumentally perturbed skin site. In one embodiment, integumental perturbation comprises disrupting the skin of the subject (for example, resulting in the induction of re-epithelialization of the skin of the subject). In some embodiments, a certain area of the epithelium is partially or wholly disrupted. In some embodiments, a certain area of both the epithelium and stratum corneum are partially or wholly disrupted. For a discussion of skin disruption and re-epithelialization, including methods for disrupting skin and inducing and detecting re-epithelialization, see PCT Publication Nos. WO2008/042216, WO 2006/105109, WO2012078649, the contents of each of which are incorporated herein by reference in their entireties. Integumental perturbation can be used to induce, for example, a burn, excision, dermabrasion, full-thickness excision, blister, or other form of abrasion or wound

Using the methods and devices described herein, integumental perturbation can be carried out in a fashion that exerts control over the extent of perturbation and/or control over the way in which the integumentally perturbed skin heals. In one embodiment, the integumental perturbation method causes only superficial wounding to the area of skin on which hair growth is desired. In a particular embodiment, the extent of wounding is minimized by controlling the depth of perturbation. For example, the integumental perturbation procedures described herein can be controlled to limit perturbation to part or all of the epidermis, to part or all of the stratum corneum, or deeper into the papillary dermis, reticular dermis, and/or hypodermis. The occurrence of pinpoint bleeding would indicate removal of the stratum corneum, epidermis (or part thereof) and portions of the upper layer of the dermis, such as the superficial papillary dermis. The occurrence of increased bleeding would indicate deeper penetration (and thus perturbation) into the deeper papillary dermis and reticular dermis layer.

Additional methods and outcomes of integumental perturbation are disclosed in International Patent Publication WO2012078649, the contents of which are herein incorporated by reference in their entirety.

In one embodiment the skin disruption is not performed by dermabrasion. In one embodiment the skin disruption is not performed using a dermaroller.

In some embodiments, integumental perturbation by one or more of the aforementioned methods is to a skin depth of between 5 and 40 μm, 40 and 100 μm and 30 and 200 μm, 50 and 150 μm, 70 and 130 μm, 80 and 120 μm, 90 and 110 μm, 95 and 105 μm, or 100 and 150 μm.

In some embodiments, integumental perturbation by one or more of the aforementioned methods is to a skin depth of at least 30 μπι. In some embodiments, integumental perturbation by one or more of the aforementioned methods is to a skin depth of 30 μπι. In some embodiments, integumental perturbation is to a skin depth of 50 um. In some embodiments, integumental perturbation by one or more of the aforementioned methods is to a skin depth of 60 μm. In some embodiments, integumental perturbation is to a skin depth of 30-100 μm. In some embodiments, integumental perturbation is to a skin depth of 60-100 μπι. In some embodiments, integumental perturbation is to a skin depth of 60-200 um. In some embodiments, integumental perturbation is to a skin depth of 100 pm. In some embodiments, integumental perturbation is to a skin depth of 100-150 μπι. In some embodiments, integumental perturbation is to a skin depth of 150 μm. In some embodiments, integumental perturbation is to a skin depth of 100-200 um. In some embodiments, integumental perturbation is to a skin depth of 30-200 μιη. In some embodiments, integumental perturbation is to a skin depth of 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μm. In some embodiments, the maximum depth of integumental perturbation is to, e.g., 30, 40, 50, 60, 70, 80, 85, 90, 95, 100, 105, 110, 120, 130, 140, 150, 160, 170, 180, 190 to 200 μm.

In some embodiments, integumental perturbation is to a skin depth of 100-500 μπι. In some embodiments, integumental perturbation is to a skin depth of less than 500 μm. In some embodiments, integumental perturbation is to a skin depth of 500-1000 μm. In some embodiments, integumental perturbation is to a maximum skin depth of about 1 mm. In some embodiments, integumental perturbation is to a skin depth of about 1 mm or more. In some embodiments, integumental perturbation is to a maximum skin depth of about 2 mm. In some embodiments, integumental perturbation is to a skin depth of about 2 mm or more. In some embodiments, integumental perturbation is to a skin depth of 1 mm to 3 mm. In some embodiments, integumental perturbation is to a skin depth of 1 mm to 5 mm. In a particular embodiment, the depth of integumental perturbation does not exceed 500 μm. In a particular embodiment, the depth of integumental perturbation does not exceed 1 mm. In a particular embodiment, the depth of integumental perturbation does not exceed 2 mm.

Any of the above-described methods may be used to disrupt a precise amount of epidermal tissue. For example, the methods of controlled integumental perturbation described herein may be used to achieve:
Disruption of the stratum corneum, disruption of the first 10-30 μm of these dead skin cells.
Disruption of the stratum corneum and part or all of the epidermis by removing the first 30-100 μm of the skin. This is not deep enough to disrupt the sebaceous gland, bulge, or hair papilla of existing follicle structures.
Disruption of the stratum corneum, all of the epidermis, and disruption of the papillary dermis (e.g., between 100 μm and 150 μm of the skin). Disruption of the papillary dermis can be detected by the appearance of small pinpoints of blood in the treated area.
Disruption of the stratum corneum, the full epidermis, and part of the dermis down to approximately 200 μm. In another embodiment, the extent of integumental perturbation and, in some cases, the resultant wounding, is reduced by controlling the size of the perturbed area of skin; for example, by making a series of small wounds to effect wounding of a large area rather than a single large wound. Thus, in certain embodiments, the area of integumental perturbation can be of any desired size, for example, between 0-3 mm in width (e.g., 1 mm, 2 mm, 3 mm, or greater), 0-2 cm in width (e.g., 1 cm, 1.5 cm, and 2.0 cm), or greater (for example, up to 10%, 30%, 50%, 70%, 90%, or 100% of a subject's scalp or other area of hair growth, such as the eyebrow area). Optionally, the area of integumental perturbation can be interfollicular.

In some embodiments of the methods described herein, the optimal functional perturbation involves perturbation of the epidermal and dermal layer. In some embodiments, the target depth is 0.8 mm. In some embodiments, the perturbation, e.g., the most productive needle strikes, create wound healing deeper in the dermis. In some embodiments, the target depth is less than 0.8 mm. In some embodiments, the target depth is greater than 0.8 mm. In some embodiments, in which the depth is greater than 0.8 mm, the wounding may occur below the dermis. In some embodiments, the wounding may include blood vessels and nerves. In some embodiments, wounding to blood vessels and nerves is avoided, e.g., through selection of a target depth equal to or less than 0.8 mm or another target depth that avoids perturbation of layers deeper than the dermis.

Topographic thickness of skin, e.g., epidermal layers, e.g., in the face or on the scalp is well known in the art (see e.g., Chopra et al. (2015), A Comprehensive examination of Topographic Thickness of Skin in the Human Face; Aesthetic Surgery Journal, 2015, Vol 35(8) 1007-1013, in particular Table 1, the contents of which are herein incorporated by reference in their entirety), and the optimal target depth can be determined accordingly. For example, the scalp epidermis on average is 35 pm. The full depth in the scalp (dermis and epidermis) is on average ˜1.2-1.5 mm, with a SD of ˜0.5 mm. Accordingly, in some embodiments, the target depth, e.g., for perturbation of the skin of the scalp, ranges from about 20 μm to 1,500 μm. In some embodiments, integumental perturbation is to a skin depth of 500-1000 μm. In some embodiments, integumental perturbation is to a skin depth of 500-600 μm. In some embodiments, integumental perturbation is to a skin depth of 600-700 μm. In some embodiments, integumental perturbation is to a skin depth of 700-800 μm. In some embodiments, integumental perturbation is to a skin depth of 700-750 μm. In some embodiments, integumental perturbation is to a skin depth of 750-800 μm. In some embodiments, integumental perturbation is to a skin depth of 750-850 μm. In some embodiments, integumental perturbation is to a skin depth of 800-850 μm. In some embodiments, integumental perturbation is to a skin depth of 850-900 μm. In some embodiments, integumental perturbation is to a skin depth of 800-900 μm. In some embodiments, integumental perturbation is to a skin depth of 900-1000 μm. In one embodiment the target depth is about 0.8 mm or 800 μm. In one embodiment the target depth is 0.8 mm or 800 μm.

In some embodiments, a method of integumental perturbation described herein induces a wound in the skin. In some such embodiments, the wounded skin is healed by primary intention. In other embodiments, the wounded skin is healed by secondary intention. In yet other embodiments, the wounded skin is healed by tertiary intention. In certain embodiments, the wounded skin is healed more slowly than usually indicated for that kind of wound. This may enhance scarless wound healing and/or prolong the period during which hair growth in the wounded area of skin is promoted.

### Needle Gauge

In some embodiments, the needles driven by the needling device are microneedles. In some embodiment the needling is microneedling. In some embodiments, the needles may be 19, 20, 21, 22, 23, 24, 25, 26, 27, 28, 29, 30, 31, 32, 33, or 34 gauge needles. combinations of the above-referenced gauges are also possible. For example, the needles may be 26-34 gauge, 28-33 gauge, 30-33 gauge, or 32 gauge.

In some embodiments, the needles may have an outer diameter of at least about 100 μm, at least about 120 μm, at least about 140 μm, at least about 160 μm, at least about 180 μm, at least about 200 μm, at least about 220 μm, at least about 235 μm, at least about 240 μm, at least about 260 μm, at least about 280 μm, at least about 300 μm, at least about 320 μm, at least about 340 μm, at least about 360 μm, at least about 380 μm, at least about 400 μm, at least about 420 μm, at least about 440 μm, at least about 460 mm, at least about 463 μm, at least about 480 μm, or at least about 500 μm. In some embodiments, the needles may have an outer diameter of less than or equal to about 500 μm, less than or equal to about 480 μm, less than or equal to about 463 μm, less than or equal to about 460 μm, less than or equal to about 440 μm, less than or equal to about 420 μm, less than or equal to about 400 μm, less than or equal to about 380 μm, less than or equal to about 360 μm, less than or equal to about 340 μm, less than or equal to about 320 μm, less than or equal to about 300 μm, less than or equal to about 280 μm, less than or equal to about 260 μm, less than or equal to about 240 μm, less than or equal to about 235 μm, less than or equal to about 220 μm, less than or equal to about 200 μm, less than or equal to about 180 μm, or less than or equal to about 160 μm. combinations of the above-referenced ranges are also possible. For example, in some embodiments, the needles may have an outer diameter of about 160 μm to about 500 μm, or about 180 μm to about 463 μm, or about 200 μm to about 400 μm, or about 200 μm to about 300 μm, or about 200 μm to about 260 μm, or about 220 μm to about 240 μm, or about 235 μm.

In some embodiments, the needles have a measureable dimension referred to herein as the needle extension length, which is the length of how far the needle extends out of the needling device (i.e. the length of the needle that is exposed and can be pierced into a subject).

In some embodiments, the needles may have a needle extension length of at least about 100 μm, at least about 500 μm, at least about 600 μm, at least about 700 μm, at least about 800 μm, at least about 900 μm, at least about 1000 μm, at least about 1100 μm, at least about 1200 μm, at least about 1500 μm, at least about 2000 μm, at least about 2000 μm, at least about 2500 μm, at least about 3000 μm, at least about 3300 μm, or at least about 4000 μm. In some embodiments, the needles may have a needle extension length of less than or equal to about 4500 μm, less than or equal to about 4000 μm, less than or equal to about 3500 μm, less than or equal to about 3000 μm, less than or equal to about 2500 μm, less than or equal to about 2000 μm, less than or equal to about 1500 μm, less than or equal to about 1400 μm, less than or equal to about 1300 μm, less than or equal to about 1200 μm, less than or equal to about 1100 μm, less than or equal to about 1000 μm, less than or equal to about 900 μm, less than or equal to about 800 μm, less than or equal to about 700 μm, less than or equal to about 600 μm, less than or equal to about 500 μm, or less than or equal to about 100 μm. combinations of the above-referenced ranges are also possible. For example, in some embodiments, the needles may have a needle extension length of about 100 μm to about 4500 μm, or about 200 μm to about 4000 μm, or about 300 μm to about 3500 μm, or about 400 μm to about 3000 μm, or about 500 μm to about 2500, or about 500 μm to about 2000, or about 600 μm to about 2000 μm, or about 600 μm to about 1600 μm, or about 600 μm to about 1400 μm, or about 600 μm to about 1300 μm, or about 600 μm to about 1200 μm, or about 600 μm to about 1100 μm, or about 600 μm to about 1000 μm, or about 700 μm to about 900 μm, or about 800 μm.

In some embodiments, the needles are hollow. In other embodiments, the needles are solid.

### Treatment Methods

The inventors have appreciated that, in order to successively balance safety and efficacy, it is important to strike the right balance between remaining adherent to minoxidil and not allowing nascent hair follicles to be diminished, and avoiding any potential safety concerns with applying minoxidil directly before skin disruption is given an opportunity to heal.

Use of the needling device is intended to selectively disrupt the dermis in a controlled manner in order to induce follicular neogenesis. While there are no data to suggest that there is higher incidence of irritation due to minoxidil application in this use setting, the inventors have developed treatment steps to mitigate risk of irritation.

According to one aspect, methods of treatment herein include initial application of 2% minoxidil topical solution (MTS) to reduce dermal irritation following needling device treatment, followed by a ramp up (or step up) to 5% MTS in later administrations.

According to another aspect, methods of treatment herein include a 24-hour minimum delay after needling device treatment prior to MTS application. In some embodiments, confirmation of wound closure is required prior to initial MTS application.

In some illustrative embodiments, two example treatment protocols are outlined as follows:

TABLE 1
Treatment Protocol with Morning Skin Disruption Treatment

| | Monday | Tuesday | Wednesday | Thursday | Friday | Saturday | Sunday |
|---|---|---|---|---|---|---|---|
| AM | Skin | 2% MTS | 2% MTS | 5% MTS | 5% MTS | 5% MTS | 5% MTS |
| | disruption | | | | | | |
| | treatment | | | | | | |
| PM | | 2% MTS | 2% MTS | 5% MTS | 5% MTS | 5% MTS | 5% MTS |

TABLE 2
Treatment Protocol with Afternoon/Evening Skin Disruption Treatment

| | Monday | Tuesday | Wednesday | Thursday | Friday | Saturday | Sunday |
|---|---|---|---|---|---|---|---|
| AM | | | 2% MTS | 2% MTS | 5% MTS | 5% MTS | 5% MTS |
| PM | Skin | 2% MTS | 2% MTS | 5% MTS | 5% MTS | 5% MTS | 5% MTS |
| | disruption | | | | | | |
| | treatment | | | | | | |

Table 1 above provides a protocol in which skin disruption treatment occurs in the morning. As shown in Table 1, starting from the morning of the day after skin disruption treatment (at least 24 hours after skin disruption treatment), 2% MTS is topically applied twice a day for the next two days for a total of 4 doses. Next, 5% MTS is topically applied twice a day for the next four days for a total of 8 doses.

Table 2 above provides a protocol in which skin disruption treatment occurs in the afternoon or evening rather than in the morning. As shown in Table 2, on the afternoon or evening of the day after skin disruption treatment (at least 24 hours after skin disruption treatment), 2% MTS is topically applied. 2% MTS is then topically applied 3 more times: twice on Wednesday and once on Thursday morning. As such, 2% MTS is applied for a total of 4 doses. On Thursday afternoon or evening, 5% MTS applied. On each subsequent day, 5% MTS is topically applied twice daily. As a result, 5% MTS is applied for a total of 7 doses.

In one embodiment, MTS, e.g., 2% MTS, is topically applied 24 hours after skin disruption treatment. In one embodiment, MTS, e.g., 2% MTS, is topically applied approximately 24 hours after skin disruption treatment. In one embodiment, MTS, e.g., 2% MTS, is topically applied 36 hours after skin disruption treatment. In one embodiment MTS, e.g., 2% MTS, is topically applied approximately 36 hours after skin disruption treatment. In one embodiment, MTS, e.g., 2% MTS, is topically applied 48 hours after skin disruption treatment. In one embodiment, MTS, e.g., 2% MTS, is topically applied approximately 48 hours after skin disruption treatment. In one embodiment, MTS, e.g., 2% MTS, is topically applied 24-36 hours (24, 25, 26, 27, 28, 29, 30, 31, 32, 33, 34, 35, 36 hours) after skin disruption treatment. In one embodiment, MTS, e.g., 2% MTS, is topically applied approximately 24 hours to approximately 36 hours after skin disruption treatment. In one embodiment, MTS, e.g., 2% MTS, is topically applied 24-48 (24, 25, 26, 27, 28, 29, 30, 31, 32, 33, 34, 35, 36, 37, 38, 39, 40, 41, 42, 43, 44, 45, 46, 47, 48 hours) after skin disruption treatment. In one embodiment, MTS, e.g., 2% MTS, is topically applied approx. 24 to approx. 48 after skin disruption treatment. In one embodiment, MTS, e.g., 2% MTS, is topically applied 36-48 hours (36, 37, 38, 39, 40, 41, 42, 43, 44, 45, 46, 47, 48 hours) after skin disruption treatment. One embodiment, MTS, e.g., 2% MTS, is topically applied approx. 36 to approx. 48 after skin disruption treatment.

In some embodiments, the time between topical applications of MTS is approximately 12 hours. In some embodiments, each dose of MTS may be approximately 1 mL. In some embodiments, confirmation that the subject's skin wounds have closed following skin disruption treatment must occur prior to application of MTS.

In some embodiments, MTS treatment, e.g., 5% MTS treatment, is discontinued 12-24 hours before the next skin disruption treatment. In some embodiments, MTS treatment, e.g., 5% MTS treatment, is discontinued approximately 24 hours before the next skin disruption treatment.

In a weekly skin disruption treatment protocol, the protocol shown in either Table 1 or Table 2 may be used and repeated every week. In some embodiments, topical application of MTS does not occur on the days on which skin disruption treatment occurs.

In a bi-weekly skin disruption treatment protocol, either the Table 1 protocol or the Table 2 protocol is carried out every other week. In the alternating weeks in which skin disruption treatment does not occur, 5% MTS solution may be applied twice daily for each day of the week. In some embodiments, the MTS solution is applied approximately every 12 hours.

Similarly, in a protocol in which skin disruption occurs once every 4 weeks, the protocols shown in either Table 1 or Table 2 is carried out once every 4 weeks. In the weeks in-between in which skin disruption treatment does not occur, 5% MTS solution may be applied twice daily for each day of the week. In some embodiments, the MTS solution is applied approximately every 12 hours.

Similarly, in a protocol in which skin disruption occurs once every 3 weeks, the protocols shown in either Table 1 or Table 2 is carried out once every 3 weeks. In the weeks in-between in which skin disruption treatment does not occur, 5% MTS solution may be applied twice daily for each day of the week. In some embodiments, the MTS solution is applied approximately every 12 hours.

In some embodiments, subjects may receive either the Table 1 protocol or the Table 2 protocol each week in which they are scheduled to receive skin disruption treatment—they need not always receive only the Table 1 protocol or only the Table 2 protocol.

However, in other embodiments, subjects may receive only the Table 1 protocol or only the Table 2 protocol, i.e., subjects that initially receive morning skin disruption treatments will always receive morning skin disruption treatments in subsequent skin disruption treatment weeks, and subjects that initially receive afternoon/evening skin disruption treatments will always receive afternoon/evening skin disruption treatments in subsequent skin disruption treatment weeks.

In some embodiments, a method for stimulating hair growth in an area of hair loss comprises the following steps:
(i) disrupting the skin in an area of hair loss;
(ii) starting approximately 24 hours following step (i), topically applying 2% minoxidil solution to the disrupted area of the skin twice daily for two (2) days;
(iii) after step (ii), topically applying 5% minoxidil solution to the disrupted area of the skin twice daily for four (4) days;
(iv) repeating steps (i)-(iii) eleven (11) times, where topical application of minoxidil does not occur on the day of step (i).

In some embodiments, the topical minoxidil in steps (ii) and (iii) is applied about every 12 hours. In some embodiments, the topical application of minoxidil, e.g., 5% topical solution, applied twice daily, is continued indefinitely.

In some embodiments, a method for stimulating hair growth in an area of hair loss comprises the following steps:
(i) disrupting the skin in an area of hair loss;
(ii) approximately 24 hours following step (i), and/or upon confirmation that the subject's skin wounds have closed, apply the first dose of 2% minoxidil to the affected skin area;
(iii) applying 3 more doses of 2% minoxidil to the affected skin area, e.g., a dose approximately every 12 hours (e.g., apply second dose of 2% minoxidil at approximately 36 hours, third dose of 2% minoxidil at approximately 48 hours, and fourth dose of 2% minoxidil at approximately 60 hours after step (i));
(iv) at approximately 72 hours after step (i), apply the first dose of 5% minoxidil to the affected skin area;
(v) applying 6 or 7 more doses of 5% minoxidil to the affected skin area, one dose twice per day, e.g., a dose approximately every 12 hours (e.g., apply second dose of 5% minoxidil at approximately 84 hours, third dose of 5% minoxidil at approximately 108 hours, fourth dose of 5% minoxidil at approximately 120 hours, fifth dose at approximately 120 hours, sixth dose at approximately 132 hours, seventh dose at approximately 144 hours, and a possible eighth dose at approximately 156 hours after step (i)); and
(vi) performing steps (i)-(v) eleven times, where minoxidil is not applied on the day of step (i).

In some embodiments, the dose of minoxidil is 1 mL. In some embodiments, the topical application of minoxidil, e.g., 5% topical solution, applied twice daily, is continued indefinitely.

Example skin disruption treatments include, but are not limited to: (i) 12 treatments, administered weekly, over 3 months, (ii) 6 treatments, administered bi-weekly, over 3 months, and (iii) 3 treatments, administered monthly, over 3 months.

In some embodiments, a method for stimulating hair growth in an area of hair loss comprises the following steps:
(i) disrupting the skin in an area of hair loss;
(ii) approximately 24 hours following step (i), topically applying 2% minoxidil solution to the disrupted area of the skin twice daily for two (2) days;
(iii) topically applying 5% minoxidil solution to the disrupted area of the skin twice daily for eleven (11) days;
(iv) repeating steps (i)-(iii) five (5) times, where minoxidil is not applied on the day of step (i).

In some embodiments, the topical minoxidil in steps (ii) and (iii) is applied about every 12 hours. In some embodiments, the topical application of minoxidil, e.g., 5% topical solution, applied twice daily, is continued indefinitely.

In some embodiments, a method for stimulating hair growth in an area of hair loss comprises the following steps:
(i) Disrupting the skin in an area of hair loss;
(ii) Approximately 24 hours following step (i), or alternatively upon confirmation that the subject's skin wounds have closed, apply the first dose of 2% minoxidil to the affected skin area;
(iii) Apply 3 more doses of 2% minoxidil to the affected skin area, e.g., a dose approximately every 12 hours (e.g., apply second dose of 2% minoxidil at approximately 36 hours, third dose of 2% minoxidil at approximately 48 hours, and fourth dose of 2% minoxidil at approximately 60 hours after step (i));
(iv) At approximately 72 hours after step (i), apply the first dose of 5% minoxidil to the affected skin area;
(v) Apply 20 or 21 more doses of 5% minoxidil to the affected skin area, e.g., a dose approximately every 12 hours (e.g., apply second dose of 5% minoxidil at approximately 84 hours, third dose of 5% minoxidil at approximately 108 hours, fourth dose of 5% minoxidil at approximately 120 hours, fifth dose at approximately 120 hours, sixth dose at approximately 132 hours, and seventh dose at approximately 144 hours after step (i), and so on for the eighth to a possible twenty-second dose); and
(vi) Perform steps (i)-(v) five times, where minoxidil is not applied on the day of step (i).

In some embodiments, the dose of minoxidil is 1 ml. In some embodiments, the topical application of minoxidil, e.g., 5% topical solution, applied twice daily, is continued indefinitely.

In some embodiments, a method for stimulating hair growth in an area of hair loss comprises the following steps:
(i) disrupting the skin in an area of hair loss;
(ii) approximately 24 hours following step (i), topically applying 2% minoxidil solution to the disrupted area of the skin twice daily for two (2) days;
(iii) topically applying 5% minoxidil solution to the disrupted area of the skin twice daily for 25 days;
(iv) repeating steps (i)-(iii) two times, where minoxidil is not applied on the day of step (i).

In some embodiments, the topical minoxidil in steps (ii) and (iii) is applied about every 12 hours. In some embodiments, the topical application of minoxidil, e.g., 5% topical solution, applied twice daily, is continued indefinitely.

In some embodiments, a method for stimulating hair growth in an area of hair loss comprises the following steps:
(i) Disrupting the skin in an area of hair loss;
(ii) Approximately 24 hours following step (i), or alternatively upon confirmation that the subject's skin wounds have closed, apply the first dose of 2% minoxidil to the affected skin area;
(iii) Apply 3 more doses of 2% minoxidil to the affected skin area, e.g., a dose approximately every 12 hours (e.g., apply second dose of 2% minoxidil at approximately 36 hours, third dose of 2% minoxidil at approximately 48 hours, and fourth dose of 2% minoxidil at approximately 60 hours after step (i));
(iv) At approximately 72 hours after step (i), apply the first dose of 5% minoxidil to the affected skin area;
(v) Apply 48 or 49 more doses of 5% minoxidil to the affected skin area, e.g., a dose approximately every 12 hours (e.g., apply second dose of 5% minoxidil at approximately 84 hours, third dose of 5% minoxidil at approximately 108 hours, fourth dose of 5% minoxidil at approximately 120 hours, fifth dose at approximately 120 hours, sixth dose at approximately 132 hours, and seventh dose at approximately 144 hours after step (i) and so on for the eighth to a possible fiftieth dose);
(vi) perform steps (i)-(v) twice, where minoxidil is not applied on the day of step (i).

In some embodiments, the dose of minoxidil is 1 ml. In some embodiments, the topical application of minoxidil, e.g., 5% topical solution, applied twice daily, is continued indefinitely.

In one embodiment, only the first round or cycle comprises a step up or ramp up from 2% to 5% minoxidil. In this specific embodiment, subsequent cycles start directly with 5% minoxidil.

### Macrophotography Procedure

A macrophotography procedure as referred to herein includes identifying the designated photography site, clipping hair within targeted photography site to a length of 1 mm, and creating a needlestick tattoo in the center of the targeted photography site for future orientation (pressure was applied until any bleeding stops), mapping the position of the tattoo, and recording the measurements on the provided documentation. The procedure further includes using the tattoo as a reference point take the first Baseline photograph without the contact plate, and again, using the tattoo as a reference point, performing remaining Baseline photography with the contact plate. From the identified designated photography site, the width of each hair is determined using an electronic caliber measurement. All hairs greater than 30 mm are classified as terminal hairs.

Parameters derived from hair measurement analysis include: Target Area Haircount (TAHC), Target Area Hair Width (TAHW), and Target Area Hair Density/Darkness (TAHD).

### Drugs/Agents and Formulations

Methods herein relate to processes in which an agent is applied to skin after the skin has been subjected to wounding from a needling device. In some embodiments, the agent is formulated in a pharmaceutical composition. In some embodiments, the pharmaceutical composition comprises the agent and a pharmaceutically acceptable hair growth-promoting agent carrier or excipient.

In some embodiments, the agent is a that may promote hair follicle development and growth. The hair growth-promoting agent may promote hair follicle development and growth which results in the transition of vellus hair on an area of the skin to nonvellus hair, e.g., intermediate or terminal, hair. In some embodiments, the hair growth-promoting agent may act synergistically with the skin disruption method to promote hair growth. The combination of the skin disruption method and the agent may promote hair follicular neogenesis. The effect that each component of the treatment offers could be an additive or synergistic improvement, or a combination of two different biologically defined effects, to achieve the desired end result.

In some embodiments, the hair growth-promoting agent is an agent that promotes hair growth and/or treats a disease or condition associated with hair loss. Any agent that promotes hair growth and/or treats a disease or condition associated with hair loss that is known in the art or yet to be developed is contemplated for use in the methods disclosed herein.

In certain embodiments, the hair growth-promoting agent may be used in its commercially available form. In other embodiments, the form of the hair growth-promoting agent is adjusted to optimize a combination treatment (e.g., skin disruption or treatment with another active ingredient) described herein. In some embodiments, the hair growth-promoting agent is formulated as a different salt form than that which is commercially available. In some embodiments, the hair growth-promoting agent is formulated for topical administration, e.g., by incorporation into a pharmaceutical composition for post-skin disruption treatment.

In some embodiments, the agent is minoxidil, which is an antihypertensive agent that opens K+ channels. In some embodiments, the agent is fluocinonide, which has anti-inflammatory properties. Other agents may be used, e.g., antiandrogens such as finasteride, dutasteride, ketoconazole, or any other suitable agents.

### Hair Growth-Promoting Agents

Most drugs for hair loss aim to retain the existing hair follicles in their active cycling states, or to rejuvenate telogen hair follicles to actively cycling anagen states. Other drugs encourage the conversion of vellus hair to terminal hair. In contrast, an integumental perturbation treatment (that may encourage the growth of “new” hair follicles or activation of existing follicles) combined with a drug treatment that may retain hair follicles in their actively cycling states, offers significant value to the individual who is balding. Such treatments may be more effective, efficient, cost-effective, and user friendly. For example, fewer treatments may be required. The hair that results may be more cosmetically satisfactory, longer lasting, thicker, more uniform, longer, and properly pigmented hair. Such characteristics are associated with terminal hair rather than vellus hair.

The integumental perturbation methods described in herein, alone or in combination with a post-perturbation treatment described in herein, may be used in combination treatments with hair growth-promoting agents, and optionally in combination with the treatments described in below (under “combinations”). In some embodiments, a hair growth-promoting agent described herein promotes hair follicle development and growth, resulting in the transition of vellus hair on an area of the skin to nonvellus, e.g., intermediate or terminal, hair. In some embodiments, a hair growth-promoting agent described herein acts synergistically with the integumental perturbation method to promote hair growth. The effect that each treatment offers could be an additive or synergistic improvement, or a combination of two different biologically defined effects, to achieve the desired end result.

In some embodiments, the hair growth-promoting agent is a treatment that promotes hair growth and/or treats a disease or condition associated with excessive hair loss. Any treatment that promotes hair growth and/or treats a disease or condition associated with excessive hair loss that is known in the art or yet to be developed is contemplated for use in accordance with these embodiments.

In some embodiments, the hair growth-promoting agent treatment comprises treatment with one or more channel openers (e.g., potassium channel opener, e.g., an ATP-sensitive potassium channel (K_{A}TP opener), or an activator of such a channel), such as, e.g., minoxidil (e.g., marketed as Rogaine or Regaine), diazoxide, or phenytoin. In a particular embodiment, the hair growth-promoting agent treatment comprises treatment with minoxidil. Commonly used dosage forms of minoxidil that may be used in accordance with these embodiments are topical solutions comprising 2% minoxidil or 5% minoxidil, for example, topical minoxidil foam 2% or 5% or topical solution 2% minoxidil or 5% minoxidil as described in the examples.

In some embodiments, the hair growth-promoting agent treatment comprises treatment with one or more 5a-reductase inhibitors. Non-limiting examples of 5a-reductase inhibitors include finasteride, dutasteride (e.g., Avodart), turosteride, bexlosteride, izonsteride, epristeride, epigallocatechin, MK-386, azelaic acid, FCE 28260, and SKF 105, 111. Commonly used dosage forms of finasteride that may be used in such treatments are, for example, oral finasteride at 1 mg day . See, e.g. , Physicians' Desk Reference, 2009, 63rd ed., Montvale, N.J.: Physicians' Desk Reference Inc., entries for Propecia® and Proscar® at pages 2095-2099 and 2102-2106, respectively, which are incorporated herein by reference in their entireties.

In some embodiments, the hair growth-promoting agent treatment comprises treatment with one or more antiandrogens, such as, e.g., finasteride (e.g., marketed as Propecia or Proscar), ketoconazole, fluconazole, spironolactone, flutamide, diazoxide, 17-alpha-hydroxyprogesterone, 11-alpha-hydroxyprogesterone, ketoconazole, RU58841, dutasteride (marketed as Avodart), fluridil, or QLT-7704, an antiandrogen oligonucleotide, or others described in Poulos & Mirmirani, 2005, Expert Opin. Investig. Drugs 14:177-184, the contents of which is incorporated herein by reference.

In some embodiments, the hair growth-promoting agent treatment comprises treatment with one or more prostaglandin F2a analogs, prostaglandin analogs, or prostaglandins. Non-limiting examples of prostaglandin F2a analogs include bimatoprost (e.g., Latisse, Lumigan), Iatanoprost (trade name Xalatan), travoprost (trade name Travatan), tafluprost, unoprostone, dinoprost (trade name Prostin F2 Alpha), AS604872, BOL303259X, PF3187207, carboprost (trade name Hemabate). For exemplary prostaglandin F2a analogs, as well as formulations, dosages, and treatment regimens, for use in accordance with the methods described herein, see, e.g., U.S. Pat. Nos. 8,017,655, 5,688,819, 6,403,649, 5,510,383, 5,631,287, 5,849,792, 5,889,052, 6,011,062, 7,163,959, 5,296,504, 5,422,368, 6,429,226, and 6,946, 120, the entire contents of each of which is incorporated herein by reference in its entirety. See also, with respect to Iatanoprost, Uno et al. , 2002, Acta Derm Venereol 82:7-12, the contents of which is incorporated herein by reference in its entirety.

In some embodiments, the hair growth-promoting agent treatment comprises treatment with one or more of the following hair growth-promoting agents: kopexil (for example, the product Keranique™), CaC , botilinum toxin A, adenosine, ketoconazole, DoxoRx, Docetaxel, FK506, GP1 1046, GP1 1511, LGD 1331, ICX-TRC, MTS-01, NEOSH101, HYG-102440, HYG-410, HYG-420, HYG-430, HYG-440, spironolactone, CB-03-01, RK-023, Abatacept, Viviscal®, MorrF, ASC-J9, NP-619, AS101, Metron-F-1, PSK 3841, Targretin (e.g., 1% gel), MedinGel, PF3187207, BOL303259X, AS604872, THGl 1331, PF-277343, PF-3004459, Raptiva, caffeine, an coffee. In some embodiments, the hair growth-promoting agent treatment comprises drugs for alopecia being developed by SWITCH Biotech LLC.

In some embodiments, the hair growth promoting agent comprises valproic acid. Use of valproic acid, e.g., post integumental perturbation is described in International Patent Application WO/2019/055662, the contents of which are herein incorporated by reference in their entirety.

In some embodiments, the hair growth-promoting agent treatment comprises treatment with one or more of the following: herbs (such as, e.g., saw palmetto, glycine soja, Panax ginseng, Castanea Sativa, Arnica Montana, Hedera Helix Geranium Maculatum), triamcinolone acetonide (e.g., suspension of 2.5 to 5 mg/ml for injection), a topical irritant (e.g., anthralin) or sensitizer (e.g., squaric acid dibutyl ester [SADBE] or diphenyl cyclopropenone [DPCP]), clomipramine, unsaturated fatty acids (e.g., gamma linolenic acid), a fatty acid derivative, thickeners (such as, e.g., carbomer, glycol distearate, cetearyl alcohol), a hair loss concealer, niacin, nicotinate esters and salts, adenosine, and methionine. In some embodiments, the hair growth-promoting agent treatment comprises treatment with nitroxide spin labels (e.g., TEMPO and TEMPOL). See U.S. Pat. No. 5,714,482, which is incorporated herein by reference.

In some embodiments, the hair growth-promoting agent treatment comprises treatment with an androgen receptor inhibitor, which have been shown to be useful for stimulating scalp hair growth (Hu LY, et al., 2007, Bioorg Med Chem Lett. 2007 17:5983-5988).

In some embodiments, the hair growth-promoting agent treatment comprises treatment with a copper peptide(s), preferably applied topically, or another compound with superoxide dismutation activity. In some embodiments, the hair growth-promoting agent treatment comprises treatment with an agent that increases nitric oxide production (e.g., arginine, citrulline, nitroglycerin, amyl nitrite, or sildenafil (Viagra)). In preferred embodiments, such compounds are administered further in combination with a catalase or catalase mimetic, or other antioxidant or free radical scavenger.

In some embodiments, the hair growth-promoting agent treatment comprises treatment with a compound that mobilizes bone marrow-derived stem cells (e.g., growth factors such as G-CSF and/or chemical agents such as plerixafor (Mozobil®)); and/or that regulates the differentiation of these stem cells into gender-specific specialized human hair follicles (e.g., using agents such as finasteride, fluconazole, spironolactone, flutamide, diazoxide, 11-alpha-hydroxyprogesterone, ketoconazole, U58841, dutasteride, fluridil, or QLT-7704, an antiandrogen oligonucleotide, cyoctol, topical progesterone, topical estrogen, cyproterone acetate, ru58841, combination 5a-reductase inhibitors, oral contraceptive pills, and others in Poulos & Mirmirani, 2005, Expert Opin. Investig. Drugs 14:177-184, incorporated herein by reference, or any other antiestrogen, an estrogen, or estrogen-like drug (alone or in combination with agents that increase stem cell plasticity; e.g., such as valproate), etc., known in the art), that can result in, e.g., the appearance of specialized follicles having features that are different from natural follicles in the target location of skin.

In some embodiments, the hair growth-promoting agent treatment comprises treatment with one or more agents that counteract age-related hair thinning and/or hair follicle cell senescence (also referred to herein as “anti-senescence agents”) for example, antioxidants such as glutathione, ascorbic acid, tocopherol, uric acid, or polyphenol antioxidants); inhibitors of reactive oxygen species (ROS) generation, such as superoxide dismutase inhibitors; stimulators of ROS breakdown, such as selenium; mTOR inhibitors, such as rapamycin; or sirtuins or activators thereof, such as resveratrol, or other SIRT1, SIRT3 activators, or nicotIn amide inhibitors.

In some embodiments, the hair growth-promoting agent treatment comprises treatment with one or more agents that induce an immune response or cause inflammation, such as, e.g., tetanus toxoid, topical non-specific irritants (anthralin), or sensitizers (squaric acid dibutyl ester [SADBE] and diphenyl cyclopropenone [DPCP]). While not intending to be bound by any theory, it is thought that by contacting these agents to the skin, lymphocytes and hair follicle stem cells may be recruited to skin. In some embodiments, the hair growth-promoting agent treatment comprises treatment with a chemical or mechanical (such as those discussed infra) treatment that induces an inflammatory process in the skin. While not intending to be bound by any theory, inducing inflammation in the site where hair growth is desired helps to recruit stem cells to the tissues that drive the formation of new follicles.

In some embodiments, the hair growth-promoting agent treatment comprises treatment with an antiapoptotic compound. In one embodiment, the antiapoptotic compound is not a Wnt or a Wnt agonist.

In some embodiments, the hair growth-promoting agent treatment comprises treatment with stem cell therapy, hair cloning, hair transplantation, scalp massage, a skin graft, hair plugs, follicular unit extraction, or any surgical procedure aimed at hair restoration.

In certain embodiments, a hair growth-promoting agent described herein may be used at a dosage or in a range of dosages known in the art for that agent (e.g., as made available on a package insert or in the Physicians ' Desk Reference). In other embodiments the regular dosage of the hair growth-promoting agent is adjusted to optimize a combination treatment (e.g., integumental perturbation or treatment with another active ingredient) described herein. For example, the regular dosage may be increased or decreased as directed by the physician. For example, a lower dosage may be used over a shorter duration owing to the synergistic effect of combination with another treatment described herein.

In certain embodiments, the hair growth-promoting agent may be used in its commercially available form. In other embodiments, the form of the hair growth-promoting agent is adjusted to optimize a combination treatment (e.g., the combination with integumental perturbation and optionally treatment with another active ingredient) described herein. In a particular embodiment, the hair growth-promoting agent is formulated as a different salt form than that which is commercially available. In a particular embodiment, the hair growth-promoting agent is formulated for topical administration, e.g., by incorporation into a pharmaceutical composition for post-perturbation treatment described in herein.

In some embodiments, the hair growth-promoting agent enhances conversion of vellus hair to nonvellus hair. In a particular embodiment, the hair growth-promoting agent enhances conversion of vellus hair to terminal hair. Exemplary hair growth-promoting agents that promote conversion of vellus to nonvellus hair that may be used in accordance with these embodiments are prostaglandin F2a analogs (in one aspect, latanoprost), minoxidil, etc. In some embodiments, the hair growth-promoting agent enhances conversion of telogen hair to anagen hair. In a particular embodiment, the hair growth-promoting agent enhances conversion of telogen hair to anagen hair. Exemplary hair growth-promoting agents that promote conversion of telogen to anagen hair that may be used in accordance with these embodiments are prostaglandin F2a analogs (in one aspect, latanoprost), minoxidil, etc.

In some embodiments, the hair growth-promoting agent treatment comprises treatment with an antiandrogen (e.g., a 5ct-reductase inhibitor) and a channel opener (e.g., minoxidil). In one such embodiment, a 5a-reductase inhibitor is administered in combination with minoxidil. In one such embodiment, finasteride is administered in combination with minoxidil. In some embodiments, the hair growth-promoting agent treatment comprises treatment with a prostaglandin F2a or prostamide analog (e.g., latanoprost, bimatoprost, etc.) in combination with a channel opener (e.g., minoxidil). In one such embodiment, a prostaglandin F2a or prostamide analog is administered in combination with minoxidil. In one such embodiment, latanoprost is administered in combination with minoxidil. In another such embodiment, bimatoprost is administered in combination with minoxidil.

In some embodiments, a treatment described herein for promoting hair growth in a female subject does not comprise finasteride or ketoconazole. In some embodiments, a treatment described herein for promoting hair growth in a pregnant female subject is not finasteride or ketoconazole.

### Compositions

In some embodiments, the agent is formulated in a pharmaceutical composition. Hair growth-promoting agents, such as minoxidil, compositions described herein comprise the corresponding acid or a pharmaceutically acceptable salt, isotopic variant, or solvate as a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. The pharmaceutical compositions described herein comprise a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. In one embodiment, each component is “pharmaceutically acceptable” in the sense of being chemically compatible with the other ingredients of a pharmaceutical formulation, and biocompatible, when in contact with the biological tissues or organs of humans and animals without excessive toxicity, irritation, allergic response, immunogenicity, or other problems or complications, commensurate with a reasonable benefit/risk ratio. See, Remington: The Science and Practice of Pharmacy, 2005, 21st ed., Philadelphia, Pa.: Lippincott Williams & Wilkins; Rowe et al, eds., 2005, Handbook of Pharmaceutical Excipients, 5th ed., The Pharmaceutical Press and the American Pharmaceutical Association; Ash & Ash eds., 2007, Handbook of Pharmaceutical Additives, 3rd ed., Gower Publishing Company; Gibson ed., 2009, Pharmaceutical Preformulation and Formulation, 2nd ed., Boca Raton, Fla.: CRC Press LLC, each of which is incorporated herein by reference.

Pharmaceutically acceptable forms of minoxidil include minoxidil sulfate, or another salt form such as chloride, carbonate, and nitrate.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, forms for topical administration such as a cream may contain excipients not suited for use in transdermal or intravenous administration. The suitability of a particular excipient depends on the specific active ingredients in the dosage form.

Commonly used dosage forms of minoxidil that may be used in accordance with the embodiments described herein are topical solutions comprising 2% minoxidil or 5% minoxidil (2% Liquid—Active—2% w/v minoxidil; Inactive—alcohol, water, propylene glycol; 5% Liquid—Active—5% w/v minoxidil; Inactive—alcohol, water, propylene glycol) or, for example, topical minoxidil foam 2 or 5%.

The pharmaceutical compositions disclosed herein may be formulated to include an appropriate aqueous vehicle, including, but not limited to, water, saline, physiological saline or buffered saline {e.g., phosphate buffered saline (PBS)), sodium chloride for injection, Ringers for injection, isotonic dextrose for injection, sterile water for injection, dextrose lactated Ringers for injection, sodium bicarbonate, or albumin for injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, lanolin oil, lanolin alcohol, linoleic acid, linolenic acid and palm seed oil. Suitable water-miscible vehicles include, but are not limited to, ethanol, wool alcohol, 1,3-butanediol, liquid polyethylene glycol {e.g., polyethylene glycol 300 and polyethylene glycol 400), propylene glycol, glycerin, N-methyl-2-pyrrolidone (NMP), N,N-dimethylacetamide (DMA), and dimethyl sulfoxide (DMSO). In one embodiment, the water-miscible vehicle is not DMSO.

Suitable isotonic agents include, but are not limited to, sodium chloride, glycerin, and dextrose. Suitable buffering agents include, but are not limited to, phosphate, glutamate and citrate. Suitable suspending and dispersing agents include but are not limited to sodium carboxymethylcelluose (CMC), hydroxypropyl methylcellulose (UPMC), polyvinyl alcohol (PVA), and polyvinylpyrrolidone (PVP). Suitable emulsifying agents include but are not limited to, including polyoxy ethylene sorbitan monolaurate, polyoxy ethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-cyclodextrin, and sulfobutylether 7-cyclodextrin (CAPTISOL®, CyDex, Lenexa, Kans.).

A product for application to the scalp or face may additionally be formulated so that it has easy rinsing, minimal skin/eye irritation, no damage to existing hair, has a thick and/or creamy feel, pleasant fragrance, low toxicity, good biodegradability, and a slightly acidic pH (pH less than 7), since a basic environment weakens the hair by breaking the disulfide bonds in hair keratin.

Provided herein are pharmaceutical compositions for administration to skin following (and optionally before or during) integumental perturbation. In certain embodiments, the pharmaceutical composition is formulated for topical administration to skin. In a particular embodiment, the pharmaceutical composition is administered to an area of the skin that will be, is being, or that has been subjected to integumental perturbation in accordance with a method described herein.

In non-limiting embodiments, a pharmaceutical composition for treatment is formulated for topical administration as a gel, hydrogel, emulsion, solution, suspension, cream, ointment, dusting powder, dressing, elixir, lotion, suspension, tincture, paste, powder, crystal, foams film, aerosol, irrigation, spray, suppository, stick, bar, ointment, bandage, wound dressing, microdermabrasion or dermabrasion particle, drop, transdermal patch, or dermal patch. In particular embodiments, the pharmaceutical composition is an aqueous formulation (e.g., hydrogel), a non-aqueous formulation, ointment, or cream (e.g., emulsion). In one embodiment, the composition is a hydrogel. In some embodiments, the composition is occlusive. In other embodiments, the composition is non-occlusive. The compositions may be administered via any topical means of delivery known in the art. In particular embodiments, the composition is administered using a drug delivery system.

In some embodiments, the formulation of the pharmaceutical composition for treatment is varied in order to control the rate of release of active ingredients (where present) in the composition. This may be accomplished by, for example, varying the molecular fluidity of the carrier, without changing its hydrophobicity, such as by varying the petrolatum to mineral oil ratio. In one embodiment, the pharmaceutical formulation is an ointment, comprising petrolatum, mineral oil, and lanolin alcohol. Exemplary formulations prepared in accordance with such embodiments are provided in the Examples below. In another embodiment, release of active ingredients can be modulated by varying the hydrophobic/hydrophilic ratio of the formulation, for example, by preparing a petrolatum/water emulsion. Exemplary formulations prepared in accordance with such embodiments are provided in the Examples below.

### Hydrogels

In one embodiment, the pharmaceutical composition for administration of the hair growth-promoting agent, e.g., minoxidil, is formulated as an aqueous hydrogel. In one embodiment, the aqueous hydrogel comprises, in addition to the hair growth-promoting agent, e.g., minoxidil, Carbopol 980, methyl paraben, propyl paraben, propylene glycol, glycerine, and water. In one embodiment, a hydrogel formulation comprises citric acid, CMC, methyl paraben, propyl paraben, allantoin, alginate, and water. Exemplary formulations prepared in accordance with such embodiments are provided in the Examples below. In one embodiment, a hydrogel has the following composition: glycerol, carboxymethyl cellulose, allantoin, sodium alginate, methyl paraben, propyl paraben, water (Q.S.), and sodium hydroxide (pH adjusted to 6.5-7.5). Methods for formulating hydrogels are described in detail in the Examples below. These methods may be adapted to generate other hydrogel formulations using methods known in the art and described herein.

In certain embodiments, a hydrogel contains approximately 75%, 80%, 85%, 90%, or 95% water. In a particular embodiment, the hydrogel contains 90% water. Preferably, the hydrogel has one or more or all of the following characteristics: is transparent, odorless, colorless, has a viscosity (at 25° C.) of, e.g., 2,000-10,000 cP, 2,000-8,000 cP, or 6,000-10,000 cP (measured using, for example, a rheometer), has assay and dose uniformity (which can be measured by, e.g., flame photometry or atomic adsorption spectrometry (AAS)), has an emollient “smooth-feel” texture, could be easily applied to skin, readily spreads over a surface, has minimal migration to surrounding sites, has minimal run off, has a neutral pH (e.g., pH 6.5-7.5), is sterile, is stable for an extended period (e.g., 1 week or more, 2 weeks or more, 4 weeks or more, 8 weeks or more, 12 weeks or more, 4 months or more, 6 months or more, 1 year or more, or 2 years or more) at one or more temperature conditions (e.g., 4° C., 25° C. and 40° C.) with respect to, for example, strength, viscosity, and homogeneity. In one embodiment, the hydrogel is stable at room temperature for up to 4 weeks or more. In one embodiment, the hydrogel is stable at room temperature for up to 8 weeks or more. In one embodiment, the hydrogel is stable at 4° C. for up to 6 months or more. In one embodiment, the hydrogel is stable at 4° C. for up to 1 year or more. In certain embodiments, a hydrogel is prepared with the excipients and an amount of active ingredient chosen to contribute to one or more of the foregoing or following attributes, which may be desirable for a topical formulation for use in the methods described herein:

In some embodiments, the hydrogel is formulated so that it releases active ingredients, where present, at varying rates. In some embodiments, most or all of the active ingredient is released from the formulation within 2 hours, within 4 hours, within 8 hours, within 10 hours, within 12 hours, within 16 hours, within 24 hours, within 36 hours, within 48 hours, within 3 days, within 5 days, within 7 days, within 10 days, within 14 days, within 30 days, or within 2 months or more. In a specific embodiment, most or all of any active ingredient is released from a hydrogel described herein within 12 hours. In one embodiment, all of the active ingredient is released from the hydrogel within 12 hours. In another embodiment, most or all of the active ingredient is released from a hydrogel described herein within 24 hours. In one embodiment, the formulation is an “immediate release” formulation, i.e., releases 90-100% of active ingredient within the first day of administration. In another embodiment, the formulation is an “Intermediate Release” formulation, i.e., releases 90-100% of active ingredient within 1 to 3 days of administration. In another embodiment, the formulation is a “Sustained Release” formulation, i.e., releases 90-100%) of active ingredient within 3 to 7 days of administration.

### Creams

In another particular embodiment, the pharmaceutical composition formulated for topical administration of the hair growth-promoting agent, e.g., minoxidil, is in the form of an emulsion, e.g., a cream. In one embodiment, the cream is an oil/water emulsion.

In certain embodiments, a cream contains approximately 75%, 80%>, 85%>, 90%, or 95%) water. In certain embodiments, the cream {e.g., dispersion, suspension, colloid or emulsion) has one or more or all of the following characteristics: is odorless, colorless upon application to the skin, has a viscosity (at 25° C.) of, e.g., 2,000-10,000 cP, 2,000-8,000 cP, or 6,000-10,000 cP (measured using, for example, a rheometer), has assay and dose uniformity (which can be measured by, e.g., flame photometry or atomic adsorption spectrometry (AAS)), has an emollient “smooth-feel” texture, could be easily applied to skin, readily spreads over a surface, has minimal migration to surrounding sites, has minimal run off, has a neutral pH {e.g., pH 6.5-7.5), is sterile, is stable for an extended period {e.g., 1 week or more, 2 weeks or more, 4 weeks or more, 8 weeks or more, 12 weeks or more, 4 months or more, 6 months or more, 1 year or more, or 2 years or more) at one or more temperature conditions {e.g., 4° C., 25° C. and 40° C.) with respect to, for example, strength, viscosity, and homogeneity. In one embodiment, the cream is stable at room temperature for up to 4 weeks or more. In one embodiment, the cream is stable at room temperature for up to 8 weeks or more. In one embodiment, the cream is stable at 4° C. for up to 6 months or more. In one embodiment, the cream is stable at 4° C. for up to 1 year or more. In certain embodiments, a cream is prepared with the excipients and an amount of active ingredient chosen to contribute to one or more of the foregoing or following attributes, which may be desirable for a topical formulation for use in the methods described herein: viscosity, surface wetting ability and prevention of “dry-out,” preservative effectiveness, maintenance of pH, stability (e.g., imparted by altering the strength of surfactants used in the cream), and pharmacokinetic properties (such as rate of release of any active ingredients from the formulation, and peak and trough concentrations in skin and blood). In embodiments where the formulation is for administration to skin that is wounded or that may be wounded, excipients that are wound compatible, contribute to wound healing, and/or aid in cell attachment and/or proliferation may be included, such as, e.g., allantoin or sodium alginate.

The rate of release of active ingredients, for example a hair growth-promoting agent, e.g., minoxidil, or a pharmaceutically acceptable salt thereof, from the cream may be modified by one or more of the following: incorporating the formulation into different scaffolds, modifying the concentration of the hair growth-promoting agent, e.g., minoxidil, or a pharmaceutically acceptable salt thereof in the formulation, or modifying the types and concentrations of excipients. For example, in one embodiment, the rate of release of active ingredients from the cream may be decreased by decreasing the concentration of hydrophilic polymers in the cream. In some embodiments, the rate of release of active ingredients from the cream may be altered by varying the concentration of cetearyl alcohol, lanolin alcohol, or by varying the types of aqueous or non-aqueous carrier(s), and preferably non-aqueous carrier(s) (e.g., silicone, mineral oil, petrolatum, etc.), used.

In some embodiments, most or all of the active ingredient is released from the formulation within 2 hours, within 4 hours, within 8 hours, within 10 hours, within 12 hours, within 16 hours, within 24 hours, within 36 hours, within 48 hours, within 3 days, within 5 days, within 7 days, within 10 days, within 14 days, within 30 days, or within 2 months or more. In a specific embodiment, most or all of the active ingredient is released from a cream described herein within 10 hours. In one embodiment, all of the active ingredient is released from the cream within 10 hours. In another embodiment, most or all of the active ingredient is released from a cream described herein within 24 hours. In one embodiment, the formulation is an “immediate release” formulation, i.e., releases 90-100% of active ingredient within the first day of administration. In another embodiment, the formulation is an “Intermediate Release” formulation, i.e., releases 90-100% of active ingredient within 1 to 3 days of administration. In another embodiment, the formulation is a “Sustained Release” formulation, i.e., releases 90-100% of active ingredient within 3 to 7 days of administration.

In a specific embodiment, the cream is an immediate release formulation. Such a formulation may be generated using a two-phase system: (i) an aqueous phase for dissolving any active ingredients and hydrophilic excipients and (ii) a non-aqueous phase for dissolving hydrophobic polymers. In an exemplary embodiment, the cream is a water-in-oil emulsion, which acts not only act as a biocompatible skin emollient, but also as a delivery system for any active ingredients. In another embodiment, the cream is an intermediate release formulation. In one embodiment, the intermediate release cream formulation is an emulsion prepared by homogenization of two phases, as described, e.g., for the immediate release cream formulation above. In another embodiment, the cream is a sustained release formulation. In one embodiment, the sustained release cream formulation is prepared by homogenization of two phases (an aqueous phase and a non-aqueous phase), as described, e.g., for the immediate and intermediate release cream formulations above, but by decreasing the concentration of hydrophilic polymers in the non-aqueous phase.

The foregoing formulations for topical administration may be administered in accordance with any embodiments described herein. For example, in specific embodiments, a 50 kg patient is administered a single droplet of a hydrogel described herein at 3 sites, twice daily. In some embodiments, the hydrogel is administered once daily. In some embodiments, the hydrogel is administered twice daily. In some embodiments of a twice daily treatment regimen, doses are administered 6 hours apart, or 7 hours apart, or 8 hours apart, or 9 hours apart, or 10 hours apart, or 11 hours apart, or 12 hours apart. In a particular embodiment, the doses are administered 7 to 8 hours apart.

### Exemplary Minoxidil Formulations

Exemplary minoxidil formulations are described in the following patent and patent applications, the contents of which are herein incorporated by reference in their entireties. Accordingly such formulations may be suitable for use in the methods described herein. Any of these formulations may be applied to a different Hair growth-promoting agent known in the art.

### Viscose Solution or Gel Formulation:

EP3250181A1: pharmaceutical formulation of minoxidil for topical use (gel or viscose solution), its use for the treatment of alopecia and relative kit for its preparation.
U.S. Pat. No. 9,326,929: non-aerosol, non-spray foam composition of minoxidil

### Aerosol Foam:

T. Wai-Chiu So, et. al., “Pharmaceutical Composition”, U.S. Pat. No. 6,946,120 B2 (Sep. 20, 2005) describe a pharmaceutical composition for topical administration including at least five percent by weight of a piperidinopyrimidine derivative or an acceptable salt thereof, an acid, a solvent composition including water, a lower alcohol and co-solvents consisting of aromatic and polyhydric alcohols, where the co-solvent includes less than approximately ten percent by weight of propylene glycol
Minoxidil Pharmaceutical Foam Formulation”, U.S. patent application no. 2005/0079139 A1 (Apr. 14, 2005) describe a pharmaceutical foam formulation in a dosage form including at least one active ingredient selected from the group consisting of minoxidil, minoxidil sulfate, other soluble minoxidil salts, a surfactant, and water; the formulation being adapted to form a foam when administered by spraying.

### Foam Spray:

E. Jacques, et. al., “Minoxidil Pharmaceutical Foam Formulation”, U.S. patent application no. 2005/0079139 A1 (Apr. 14, 2005) describe a pharmaceutical foam formulation in a dosage form including at least one active ingredient selected from the group consisting of minoxidil, minoxidil sulfate, other soluble minoxidil salts, a surfactant, and water; the formulation being adapted to form a foam when administered by spraying.
Exemplary minoxidil aerosol formulations, such a dry shampoo formulations, are described in US20160220465, the contents of which are herein incorporated by reference in their entirety.

### Aerosol Liquid Spray:

P. Cronk, et. al., “Continuous Spray Scalp Therapy and Dispensing Systems for Same”, U.S. patent application no. 2008/0206156 A1 (Aug. 28, 2008) describes continuous spray medications, spray medication dispensing systems, and methods for treating alopecia, in which a continuous mist of a scalp medication, such as minoxidil, finesteride, copper peptides, DHT inhibitors and/or androgen receptor blockers, disposed within a pharmacologically acceptable carrier solution, is administered in an amount sufficient to stimulate or maintain hair growth.
P. Uster, et. al., “Non-crystalline Minoxidil Composition”, U.S. Pat. No. 5,030,442 (Jul. 9, 1991) describe an aqueous, non-crystalline minoxidil composition for topical use containing minoxidil complexed with an amphipathic compound, oleic acid, and acceptable excipients, which has improved flux through human cadaver skin and is formulated in an aqueous vehicle or dispersed in fluorochlorocarbon solvent for spray delivery.
P. Uster, et. al., “Non-crystalline Minoxidil Composition, its Production and Application”, U.S. Pat. No. 4,828,837 (May 9, 1989) describe an aqueous, non-crystalline minoxidil composition for topical use containing minoxidil complexed with an amphipathic compound containing a single lipophilic chain moiety and a sulfate, sulfonate, phosphate and phosphonate polar moiety, and having a pK less than five. The composition is formulated in ointment form, in an aqueous vehicle, or dispersed in a fluorochlorocarbon solvent for spray delivery.

### Aqueous Vehicle:

J. Cappello, “Topical and Transdermal Treatments Using Urea Formulation”, U.S. Pat. No. 7,803,357 B2 (Sep. 28, 2010) describes a topical application of a composition of urea and a chemotherapeutic agent such as sclerosing agents, vasodilators, botulinum toxin, and minoxidil that is effective in treating spider veins, erectile dysfunction, facial wrinkles, hair loss, and baldness.
S. Malek, “Topical Administration Carrier Composition and Therapeutic Formulations Comprising Same”, U.S. Pat. No. 7,749,489 B2 (Jul. 6, 2010) describes a topically administered carrier composition including water, glycerin, and polysorbate, which retards the evaporative losses of the solvent component and systemic migration losses of the active ingredient to provide sustained topical action for use in formulations containing active ingredients, such as minoxidil.
K. Hallam, et. al., “Methods and Compositions for the Promotion of Hair Growth”, U.S. Pat. No. 6,465,514 B1 (Oct. 15, 2002) describe compositions, medicaments, and methods for the promotion of hair growth, topically applied to the scalp by use of an eyedropper or other suitable means, including either local anesthetics of the secondary and tertiary amino type and/or niacin. The compositions and medicaments include minoxidil and either procaine hydrochloride or niacin or procaine hydrochloride and niacin in a non-sulfur-containing carrier.
C. Chidsey, III, et. al., “Methods and Solutions for Treating Male Pattern Alopecia”, U.S. Pat. No. 4,596,812 (Jun. 24, 1986) describe a method for treating male pattern baldness, which includes regular topical application to the human scalp of a composition containing 6-amino-1,2-dihydro-1-hydroxy-2-imino-4-piperidinopyrimidine as one of its active ingredients.

### Gel/Ointment/Cream

L. Pena, et. al., “Compositions of Minoxidil”, U.S. Pat. No. 7,442,369 B1 (Oct. 28, 2008) describe novel compositions comprising minoxidil, a thickening agent, and a pharmaceutically acceptable solvent; a process for making a gel composition including minoxidil; and methods for using the compositions for treating and preventing hair loss in a patient.
C. Samour, “Method for Treating Hair Loss”, U.S. Pat. No. 5,620,980 (Apr. 15, 1997) describes the combination of minoxidil and 2-n-nonyl-1,3-dioxolane for promoting hair growth when applied once daily.
L. Pena, “Minoxidil Gel”, U.S. Pat. No. 5,225,189 (Jul. 6, 1993) describes a pharmaceutical gel containing minoxidil for topical application.
J. Grollier, “Composition in the Form of a Gel for Inducing and Stimulating Hair Growth and for Decreasing Their Loss, Based on Piperidinopyrimidine Derivatives”, U.S. Pat. No. 4,820,512 (Apr. 11, 1989) describes a gel composition for inducing and stimulating hair growth and for reducing hair loss based on piperidinopyrimidine derivatives.
   Oral in Conjunction with Topical Application
C. Catalfo, et. al., “Compositions Containing Minoxidil and Saw Palmetto for Treating Baldness”, U.S. Pat. No. 6,596,266 B2 (Jul. 22, 2003) describe compositions containing minoxidil as an active ingredient, other active agents and/or enhancer agents, such as saw palmetto extract and nettle root extract, and methods of using the compositions to treat male patterned baldness and to stimulate hair growth on the scalp.

### Encapsulation

N. Weiner, et. al., “Stimulation of Hair Follicles”, U.S. Pat. No. 5,834,014 (Nov. 10, 1998) describe a novel method and delivery system for the topical delivery of a therapeutic weak acid or a base material, such as minoxidil, that utilizes a therapeutic material, which is modified to make it more hydrophilic, encapsulated in a lipid vesicle, preferably a non-phospholipid vesicle.

### Formulation and Apparatus:

N. Maniga “Method and apparatus for delivery of a measured dosage of a non-aerosol, non-spray foam composition of minoxidil U.S. Pat. No. 9,326,929: A non-pressurized container that contains one or more of: a foamable composition of minoxidil, or a pharmaceutically acceptable salt thereof, and a dihydrotestosterone blocker in a formulation with a precise density and viscosity to yield a foam with a temperature sensitivity and shear strength designed for inverted application and immediate targeted release at body temperature under a minimal hand applied pressure to the scalp.

### Hydrogel:

Lu S. et al., “Reversely Thermo-Reversible Hydrogel Compositions” US Patent Application No. 20150071864: A reversely thermo-reversible hydrogel composition comprising a water soluble block copolymer comprising at least two blocks of polyethylene oxide and at least one block of polypropylene oxide, and at least one associative gelling adjuvant having water solubility less than 0.5 g/100 ml, preferably less than 0.3 g/100 ml at 20° C., and being capable of forming water soluble inter-molecular complexes with the water soluble block copolymer in water. The hydrogel composition exhibits improved gelling efficiency, enhanced solubility and/or stability for water sparely soluble and insoluble pharmaceutical agents, useful for treating and/or preventing alopecia and restoring and/or promoting hair growth

### Controlled Release:

Malek, Shane; Exemplary controlled release formulations for minoxidil is described in US Patent Publication US20180177878, a formulation comprising a release composition that mediates a dermal flux of minoxidil of at least 1.5 μg minoxidil/cm2 skin area/hour for a time of from 17 to 27 hours, at an applied dose of from 2 to 20 mg minoxidil to said skin region, and with a period of from 16 to 30 hours before any subsequent reapplication of the formulation

### Combinations

The methods and treatment regimens described herein can be applied before, concurrently, after, or alternating with one or more additional hair growth-promoting agents.

Hair growth-promoting agents for use, alone or in combination, in accordance with this aspect include but are not limited to: agents affecting prostaglandins, such as Prostaglandin F2a analogs, e.g. latanoprost (trade name Xalatan), travoprost (trade name Travatan), tafluprost, unoprostone, dinoprost (trade name Prostin F2 Alpha), AS604872, BOL303259X, PF3187207, carboprost (trade name Hemabate); Prostamides, e.g., bimatoprost (trade names Latisse, Lumigan); Prostanoid receptor agonists, e.g. fluprostenol; Prostaglandin D2 receptor antagonists, e.g. laropiprant, AM211; Prostglandin E2 analogs, e.g. sulprostone; and EP 2 receptor agonists, e.g. butaprost; 5<x-reductase inhibitors, such as, e.g., finasteride, dutasteride, turosteride, bexlosteride, izonsteride, epristeride, epigallocatechin, Fluridil (Sovak et al, Dermatol Surg. 2002; 28(8):678-685), RU 58841 (Pan et al. Endocrine. 1998; 9(1):39-43), N,N-diethyl-4-methyl-3-oxo-4-aza-5 alpha-androstane-17 beta-carboxamide (Rittmaster et al., J Clin Endocrinol Metab. 1987; 65(1):188-193), MK-386, azelaic acid, FCE 28260, SKF 105,111; Minoxidil; ATP-sensitive potassium channel openers, e.g. diazoxide; and the hair growth-promoting agents described herein or otherwise known in the art, such as, e.g., kopexil (for example, the product Keranique™), CaC, botilinum toxin A, adenosine, ketoconazole, DoxoRx, Docetaxel, FK506, GP1 1046, GP11511, LGD 1331, ICX-TRC, MTS-01, NEOSH1O1 , HYG-102440, HYG-410, HYG-420, HYG-430, HYG-440, spironolactone, CB-03-01, RK-023, Abatacept, Viviscal®, MorrF, ASC-J9, NP-619, AS101, Metron-F-1, PS 3841, Targretin (e.g., 1% gel), MedinGel, PF3187207, BOL303259X, AS604872, THG11331, PF-277343, PF-3004459, Raptiva, caffeine, and coffee. Other hair-growth promoting agents include arginine, isoleucine, leucine, lysine, methionine, phenylalanine, threonine, tryptophan, valine, gamma linoleic acid and polyphenol catechins, copper peptides. Other hair-growth promoting agents that can be formulated as a hair wash tonic could include but are not limited to, jojoba oil, extract of apple, saw palmetto, emu oil, beta carotene and green tea. In one aspect, an integumental perturbation method of the invention is used in combination with drugs for alopecia being developed by SWITCH Biotech LLC.

In some embodiments, a method of the invention comprising integumental perturbation and administration of a pharmaceutical composition comprising one or more hair growth-promoting agents may be combined with one or more additional treatments with an active ingredient. In certain embodiments, additional the active ingredient is administered together with, optionally in the same formulation, a the hair growth promoting composition.

In accordance with these embodiments, an additional active ingredient for formulation into a pharmaceutical composition for an additional treatment can be selected from, e.g., cell therapy (such as a stem cell), a formulation for gene therapy (such as, e.g. , a virus, virus-like particle, virosome), an antibody or antigen-binding fragment thereof, an herb, a vitamin (e.g., a form of vitamin E, a vitamin A derivative, such as, e.g. , all-trans retinoic acid (ATRA), a B vitamin, such as, e.g., inositol, panthenol, or biotin, or a vitamin D3 analog), a mineral, essential oils, an antioxidant or free radical scavenger, amino acids or amino acid derivatives, a shampoo ingredient (e.g., caffeine, ammonium chloride, ammonium lauryl sulfate, glycol, sodium laureth sulfate, sodium lauryl sulfate, ketoconazole, zinc pyrithione, selenium sulfide, coal tar, a salicylate derivative, dimethicone, or plant extracts or oils), a conditioning agent, a soap product, a moisturizer, a sunscreen, a waterproofing agent, a powder, talc, or silica, an oil-control agent, alpha-hydroxy acids, beta-hydroxy acids (e.g., salicylic acid), poly-hydroxy acids, benzoyl peroxide, antiperspirant ingredients, such as astringent salts (e.g. , zinc salts, such as zinc pyrithione, inorganic or organic salts of aluminum, zirconium, zinc, and mixtures thereof, aluminum chloride, aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum sulfate, aluminum zirconium octachlorohydrate, aluminum zirconium octachlorohydrex GLY (abbreviation for glycine), aluminum zirconium pentachlorohydrate, aluminum zirconium pentachlorohydrex GLY, aluminum zirconium tetrachlorohydrate, aluminum zirconium trichlorohydrate, aluminum zirconium tetrachlorohydrate GLY, and aluminum zirconium trichlorohydrate GLY, potassium aluminum sulphate, (also known as alum (KAl(S04)212H20)), aluminum undecylenoyl collagen amino acid, sodium aluminum lactate* aluminum sulphate (Na₂HAl(OOCCHOHCH₃)2—(OH)₆)+Al₂(SO₄)3), sodium aluminum chlorohydroxylactate, aluminum bromohydrate (Al₂Br(OH)₅ₙ¾0), aluminum chloride (Al C¾6H20), complexes of zinc salt and of sodium salt, complexes of lanthanum and cerium, and the aluminum salt of lipoamino acids (R—CO—NH—CUR—CO—OAl—(OH)₂ with R=C₆₋ᵢᵢ and R′=amino acid), retinoids (e.g., retinoic acid, retinol, retinal, or retinyl esters), sunscreens (e.g., derivatives of para-aminobenzoic acid (PABA), cinnamate and salicylate, avobenzophenone (Parsol 1789®), octyl methoxycinnamate (Parsol™ MCX) and 2-hydroxy-4-methoxy benzophenone (also known as oxybenzone and available as Benzophenone™, and preservatives), an anti-age agent, a sebum production inhibitor and/or pore size reducing agent (e.g., carboxyalkylates of branched alcohols and/or alkoxylates thereof, e.g., tridecyl carboxy alkylates, cerulenin or a cerulenin analog, including pharmaceutically acceptable salts or solvates thereof, another fatty acid synthase inhibitor, such as triclosan or analogs thereof, a polyphenol extracted from green tea (EGCG), available from Sigma Corporation (St. Louis, Mo.), or a-methylene-y-butyrolactone), a massage agent, an exfoliant, an anti-itch agent, a pro-inflammatory agent, an immunostimulant (e.g., cytokines, agonists or antagonists of various ligands, receptors and signal transduction molecules of the immune system, immunostimulatory nucleic acids, an adjuvant that stimulates the immune response and/or which causes a depot effect), a cell cycle regulator, a hormonal agonist, hormonal antagonist (e.g., flutamide, bicalutamide, tamoxifen, raloxifene, leuprolide acetate (LUPRON), LH-RH antagonists), an inhibitor of hormone biosynthesis and processing, a steroid (e.g., dexamethasone, retinoids, deltoids, betamethasone, Cortisol, cortisone, prednisone, dehydrotestosterone, glucocorticoids, mineralocorticoids, estrogen, testosterone, progestins), antigestagens (e.g., mifepristone, onapristone), an antiandrogen (e.g., cyproterone acetate), an antiestrogen, an antihistamine (e.g., mepyramine, diphenhydramine, and antazoline), an anti-inflammatory (e.g., corticosteroids, NTHEs, and COX-2 inhibitors), a retinoid (e.g., 13-cis-retinoic acid, adapalene, all-trans-retinoic acid, and etretinate), an immunosuppressant (e.g., cyclosporine, tacrolimus, rapamycin, everolimus, and pimecrolimus), an antibiotic, an anti-cancer agent (such as, e.g., fluorouracil (5-FU or f5U) or other pyrimidine analogs, methotrexate, cyclophosphamide, vincristine), a mood stabilizer (e.g., valproic acid or carbamazepine) an antimetabolite, an anti-viral agent, and an antimicrobial (e.g., benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal (thimerosal)).

In one embodiment, the hair promoting agent is Minoxidil, Minoxidil sulfate, or another salt form such as chloride, carbonate, nitrate, etc. In another embodiment, the hair promoting agent or additional combination agent is a prostaglandin, or a prostaglandin analogue, a prostaglandin prodrug. In another embodiment, the drug is a 5 alpha reductase inhibitor.

In some embodiments, the additional treatment comprises one or more antibiotics or antimicrobial agents or preservatives including, but not limited to, alkyl esters of p-hydroxybenzoic acid, hydantoins derivatives, propionate salts, phenols, cresols, mercurials, phenyoxyethanol, benzyl alcohol, chlorobutanol, methyl and propyl p-hydroxybenzoates, thimerosal, benzalkonium chloride (e.g., benzethonium chloride), butyl, methyl- and propylparabens, sorbic acid, and any of a variety of quarternary ammonium compounds. Suitable antioxidants include ascorbate, bisulfite and sodium metabisulfite.

Additional agents which can be combined with hair growth promoting agents include hydroxychloroquine, doxycycline, mycophenolate mofetil, cyclosporine, or corticosteroids; topical drugs include corticosteroids, tacrolimus, or pimecrolimus; and triamcinolone acetonide.

In some embodiments, the methods and treatment regimens described herein include the application of a steroid, e.g., provided in combination with the hair growth promoting agent. Non-limiting examples of steroids include corticoids, dexamethasone, retinoids, deltoids, betamethasone, Cortisol, cortisone, prednisone, dehydrotestosterone, glucocorticoids, mineralocorticoids, estrogen, testosterone, and progestins. Additional non-limiting examples of steroids include Augmented betamethasone dipropionate (Diprolene), Clobetasol propionate (Clobex, Olux, Temovate), Diflorasone diacetate (Apexicon), Fluocinonide (Vanos), Flurandrenolide (Cordran), Halobetasol propionate (Ultravate), Amcinonide, Augmented betamethasone dipropionate, Diprolene, Diprolene AF, Betamethasone dipropionate, Diprosone, Desoximetasone, Topicort, Topicort, Diflorasone diacetate (Apexicon E), Fluocinonide (Lidex), Halcinonide, Amcinonide, Betamethasone dipropionate, (Betanate), Fluticasone propionate (Cutivate), Triamcinolone acetonide (Cinalog), (Betamethasone valerate (Beta-Val, Luxiq), Desoximetasone (Topicort), Fluocinolone acetonide (Synalar), Fluticasone propionate (Cutivate), Hydrocortisone butyrate (Locoid), Hydrocortisone probutate (Pandel), Hydrocortisone valerate (Westcort), Mometasone furoate (Elocon), Triamcinolone acetonide (Kenalog), Triamcinolone acetonide (Triderm), Alclometasone dipropionate, (Aclovate), Desonide, Desonate, (Desowen, Verdeso), Fluocinolone, Hydrocortisone butyrate (Locoid), and Hydrocortisone.

Non-limiting examples of formulations include augmented betamethasone dipropionate 0.05% (Diprolene), Clobetasol propionate 0.05% (Clobex, Olux, Temovate), Diflorasone diacetate 0.05% (Apexicon), Fluocinonide 0.1% (Vanos), Flurandrenolide 4 mcg per m2 (Cordran), Halobetasol propionate 0.05% (Ultravate), Amcinonide 0.1%, Augmented betamethasone dipropionate 0.05%, Diprolene, Diprolene AF, Betamethasone dipropionate 0.05%, Diprosone, Desoximetasone, Topicort 0.25%, Topicort 0.05%, Diflorasone diacetate 0.05% (Apexicon E), Fluocinonide 0.05% (Lidex), Halcinonide 0.1%, Amcinonide 0.1%, Betamethasone dipropionate 0.05%, (Betanate), Fluticasone propionate 0.005% (Cutivate), Triamcinolone acetonide 0.5% (Cinalog), (Betamethasone valerate (Beta-Val 0.1%, Luxiq 0.12%), Desoximetasone 0.05% (Topicort), Fluocinolone acetonide 0.025% (Synalar), Fluticasone propionate 0.05% (Cutivate), Hydrocortisone butyrate 0.1% (Locoid), Hydrocortisone probutate 0.1% (Pandel), Hydrocortisone valerate 0.2% (Westcort), Mometasone furoate 0.1% (Elocon), Triamcinolone acetonide 0.025% (Kenalog), Triamcinolone acetonide 0.1% (Triderm), Alclometasone dipropionate 0.05%, (Aclovate), Desonide 0.05%, Desonate, (Desowen, Verdeso), Fluocinolone 0.01%, Hydrocortisone butyrate 0.1% (Locoid), Hydrocortisone 1%, 2.5%.

In some embodiments, the methods and treatment regimens described herein include the application of an antibiotic known in the art. Non-limiting examples for topical administration include Suitable antibiotics are known in the art, and non-limiting examples of antibiotics further include bacitracin, bacitracin/polymyxin B, double antibiotic chlorhexidine gluconate, gentamicin, hydrogen peroxide, iodine, isopropyl alcohol, mafenide, Sulfamylon mupirocin, Bactroban, PA , neomycin/bacitracin/polymyxin B/hydrocortisone combination, Cortisporin, neomycin/bacitracin/polymyxin, triple antibiotic neomycin/polymyxin B/hydrocortisone, silver sulfadiazine-Silvadene—SSD, and silver sulfadiazine-Thermazene. In some embodiments, antibiotics are provided orally or intravenously.

In some embodiments, the combination treatment for enhancing hair growth in a patient having scarring alopecia comprises integumental perturbation, optionally in combination with a hair growth-promoting agent treatment, in combination with one or more antibiotics, such as oral or topical antibiotics. In some embodiments, the combination treatment comprises one or more retinoids, such as isotretinoin, or methotrexate, tacrolimus, cyclosporin, or thalidomide. In particular, non-limiting embodiments, such combinations are used in enhancing hair growth in a patient with the neutrophilic group of cicatricial alopecias (e.g., folliculitis decalvans, tufted folliculitis, and dissecting cellulitis), and successful treatment enhances hair growth while reducing or eliminating microbes that are involved in the inflammatory process.

In some embodiments, the additional treatment comprises one or more pain relievers, e.g., non-steroidal anti-inflammatory drugs or acetaminophen. In some embodiments, the additional treatment comprises one or more narcotic analgesics, selected from the group of, e.g., alfentanil, benzylmorphine, codeine, codeine methyl bromide; codeine phosphate, codeine sulfate, desomorphine, dihydrocodeine, dihydrocodeinone enol acetate, dihydromorphine, ethylmorphine, hydrocodone, hydromorphone, methadone hydrochloride, morphine, morphine hydrochloride, morphine sulfate, nicomorphine, normethadone, normorphine, opium, oxycodone, oxymorphone, phenoperidine, and propiram. In some embodiments, the additional treatment comprises one or more non-narcotic analgesics, selected from the group of, e.g., aceclofenac, acetaminophen, acetanilide, acetylsalicylsalicylic acid; aspirin, carbamazepine, dihydroxyalumimsm acetylsalicylate, fenoprofen, fluproquazone, ibufenac, indomethacin, ketorolac, magnesium acetylsalicylate, morpholine salicylate, naproxen, phenacetin, phenyl salicylate, salacetamide, salicin, salicylamide, sodium salicylate, and tolfenamic acid. Other pain treatments that may be used include nerve blocks or non-traditional pain medications, such as, e.g., Lyrica (pregabalin) or Neurontin (gabapentin).

In some embodiments, the additional treatment comprises one or more analgesic compounds. Analgesic compounds can be administered topically, by injection directly to the site of the disruption, or systemically. Examples of analgesic compounds for use in the methods and compositions described herein include lidocaine, prilocaine, bupivicaine, levobupivacaine, ropivacaine, mepivacaine, dibucaine, isoflurane, desflurane, sevoflurane, and nitrous oxide. Suitable local analgesics include, but are not limited to, procaine hydrochloride, lidocaine and salts thereof, benzocaine and salts thereof and novacaine and salts thereof.

Soothing preparations, e.g., for topical administration, may contain sodium bicarbonate (baking soda), and coal tar based products. Formulations may also optionally contain a sunscreen or other skin protectant, or a waterproofing agent.

In accordance with this aspect, one or more of the foregoing may be used in its commercially available form. In other aspects, the dosage of one or more of the foregoing is adjusted to optimize a combination treatment (e.g., integumental perturbation or treatment with another active ingredient or active ingredients) described herein. In other aspects, the formulation of one or more of the foregoing is adjusted to optimize a combination treatment (e.g. , integumental perturbation or treatment with another active ingredient or active ingredients) described herein. In a particular aspect, one or more of the foregoing is formulated for topical administration, e.g., by incorporation into a pharmaceutical composition for post-perturbation treatment, as described herein.

In some embodiments, these combination treatments are topically administered. In some embodiments, the treatments are administered via an alternate route, e.g., oral, injection (subcutaneous, intramuscular, or intravenous route), patch, implantation, or any other route known in the art.

### Administration of Hair Growth-Promoting Agents

In some embodiments, the methods described herein comprises administration (e.g., topical administration) of a hair growth promoting agent, e.g., minoxidil (e.g., a pharmaceutical composition comprising the agent, e.g., minoxidil, or a pharmaceutically acceptable salt, isotopic variant, or solvate thereof, that is intended to promote the growth of hair, including vellus hair, non-vellus hair, intermediate hair, terminal hair, increase hair thickness, prevent infection and/or promote healing, e.g., scarless healing, of the perturbed skin.

In some embodiments, the hair growth promoting agent or a pharmaceutically acceptable salt thereof is present in an amount of 0.5-30% wt % based on the total weight of the pharmaceutical composition. In some embodiments, the hair growth promoting agent or a pharmaceutically acceptable salt thereof is present in an amount of 2.0-25% wt % based on the total weight of the pharmaceutical composition. In some embodiments, the hair growth promoting agent or a pharmaceutically acceptable salt thereof is present in an amount of 0.5-1, 1-1.5, 1.5-2, 2-2.5, 2.5-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-21, 21-22, 22-23, 23-24, 24-25, or 25% wt % based on the total weight of the pharmaceutical composition. In some embodiments, the hair growth promoting agent or a pharmaceutically acceptable salt thereof is present at least in an amount of 0.5-1, 1-2, 2-3, 3-4, 4-5, 5-6, 6-7, 7-8, 8-9, 9-10, 10-15, 15-20, or 20% wt % based on the total weight of the pharmaceutical composition. In some embodiments, the hair growth promoting agent or a pharmaceutically acceptable salt thereof is present in an amount of 0.5, 1, 1.5, 2, 2.5, 3, 4, 5, 6, 7, 8, 9, 10, 11, 12, 13, 14, 15, 16, 17, 18, 19, 20, 21, 22, 23, 24, or 25% wt % based on the total weight of the pharmaceutical composition. In some embodiments, the hair growth promoting agent or a pharmaceutically acceptable salt thereof is present at least in an amount of 0.5, 1, 2, 3, 4, 5, 6, 7, 8, 9, 10, 15, or 20% wt % based on the total weight of the pharmaceutical composition. In some embodiments, the hair growth promoting agent or a pharmaceutically acceptable salt thereof is present at least in an amount 0.5% wt % based on the total weight of the pharmaceutical composition. In some embodiments, the hair growth promoting agent or a pharmaceutically acceptable salt thereof is present at least in an amount 1% wt % based on the total weight of the pharmaceutical composition. In some embodiments, the hair growth promoting agent or a pharmaceutically acceptable salt thereof is present at least in an amount 5% wt % based on the total weight of the pharmaceutical composition.

In some embodiments, minoxidil or a pharmaceutically acceptable salt thereof is present in an amount of 0.5-10% wt % based on the total weight of the pharmaceutical composition. In some embodiments, the minoxidil or a pharmaceutically acceptable salt thereof is present in an amount of 0.5-1, 1-1.5, 1.5-2, 2-2.5, 2.5-3, 3-4, 4-5, 5-6, 6-7, 7-8, 8-9, 9-10% wt % based on the total weight of the pharmaceutical composition. In some embodiments, the minoxidil or a pharmaceutically acceptable salt thereof is present in an amount of 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3,3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11 wt % based on the total weight of the pharmaceutical composition. In some embodiments, the minoxidil or a pharmaceutically acceptable salt thereof is present at least in an amount 2% wt % based on the total weight of the pharmaceutical composition. In some embodiments, the minoxidil or a pharmaceutically acceptable salt thereof is present at least in an amount 5% wt % based on the total weight of the pharmaceutical composition.

Any of these pharmaceutical compositions in which minoxidil a pharmaceutically acceptable salt thereof is present at amounts wt % described above can be used in the methods described herein, either as the initial dose or as the step up dose.

### Glossary and Definitions

The following terms are used herein consistently with their art-accepted meanings summarized below.

### Alopecia: Abnormal Hair Loss:

Alopecia areata: Hair loss in patches, thought to be caused by an autoimmune response to hair follicles in the anagen stage; extensive forms of the disorder are called alopecia areata totalis (hair loss over the entire scalp) and alopecia areata universalis (hair loss over the entire body).
Androgenetic alopecia (AGA): Baldness caused by miniaturization of genetically predisposed follicles in the MPHL pattern (frontal recession and thinning at the vertex) or the FPHL pattern (loss of hair primarily over the crown, with sparing of frontal hair).
Female Pattern Hair Loss (FPHL): form of gender specific hair patterning in females (also sometimes referred to as female pattern alopecia).
Integumental: Pertaining to the integumentary system, which comprises the skin (epidermis, dermis, hypodermis (or subcutanea)) and all cells contained therein regardless of origin, and its appendages (including, e.g., hair and nails).
Intermediate hair: A subset of “terminal hair”. Hair shaft diameters typically 30 μπι or greater, but less than 60 μπι.
Male Pattern Hair Loss (MPHL): form of gender specific hair patterning in men (also sometimes referred to as male pattern alopecia).
Nonvellus hair: Same as “terminal hair.”
Permanent alopecia: Caused by destruction of hair follicles as a result of inflammation, trauma, fibrosis, or unknown causes; examples include lichen planopilaris and discoid lupus erythematosus. Includes diseases referred to as scarring alopecia.
Terminal hair: Large, usually pigmented hairs on scalp and body. Hair shaft diameters typically 30 μm or greater.
Vellus hair: Very short, often nonpigmented hairs (e.g., those found diffusely over nonbeard area of face and bald scalp as a result of miniaturization of terminal hairs). In certain embodiments, as used herein, a “vellus” hair is a hair that is less than 2 mm in length and less than 30 μηι in diameter. In certain embodiments, as used herein, a “vellus” hair is a hair that is determined histologically as having a hair shaft diameter of less than 30 μm and not exceeding the thickness of its surrounding internal root sheath.

Herein, the terms “disruption” or “skin disruption” and “integumental perturbation” are used synonymously. Accordingly, as used herein, the termes “disrupted” and “perturbed” have the same meaning.

As defined herein, with respect to a recited hourly time, the term “approximately” means any time within a four (4) hour window before or after the recited hour. Therefore, for example the term “approximately 12 hours” means 8-16 hours (any hourly and minute increments within the 8-16 hour time frame). For example, “approximately 12 hours” means 8 hours, 9 hours, 10 hours, 11 hours, 12 hours, 13 hours, 14 hours, 15 hours, and 16 hours, and any incremental minute within the recited hourly increments. For example, the term “approximately 24 hours” means 20-28 hours (any hourly and minute increments within the 20-28 hour time frame). For example, “approximately 24 hours” means 20 hours, 21 hours, 22 hours, 23 hours, 24 hours, 25 hours, 26 hours, 27 hours, and 28 hours, and any incremental minute within the recited hourly increments. The term “approximately 36 hours” means 32-40 hours (any hourly and minute increments within the 32-40 hour time frame). For example, “approximately 36 hours” means 32 hours, 33 hours, 34 hours, 35 hours, 36 hours, 37 hours, 38 hours, 39 hours, and 40 hours, and any incremental minute within the recited hourly increments. The term “approximately 48 hours” means 44-52 hours (any hourly and minute increments within the 44-52 hour time frame). For example, “approximately 48 hours” means 44 hours, 45 hours, 46 hours, 47 hours, 48 hours, 49 hours, 50 hours, 51 hours, and 52 hours, and any incremental minute within the recited hourly increments.

With respect to the term “day”, it is meant that a day means a 24 hour period. Is does not necessarily have to be a calendar day. A day can encompass two consecutive calendar days if they are within the same 24 hour period. For example, “a day” as meant herein can be 4 pm calendar day 1 to 4 pm calendar day 2.

### EXAMPLES

### Example 1

### Experimental Design

A multicenter randomized study was conducted to assess the safety and efficacy of treatment methods using a needling device in conjunction with minoxidil in adult males with androgenetic alopecia (AGA). The study involved 14 subjects, male, age 18-40 years, with a diagnosis of androgenetic alopecia. Data for 46 additional subjects is being gathered. The subjects were randomized and then completed treatment across 12 weeks (total of 3, 6, or 12 treatments with the needling device per randomization assignment, n=20 per arm). A final evaluation was conducted on Day 85 with no additional follow up required.

The needling device used is a powered needling device that drives a plurality of microneedles in a reciprocating motion to produce small wounds in skin, similar to the embodiment shown and described in the FIGS. 96-103 of U.S. Published Application No. 2018/0280675.

A macrophotography procedure as described above was carried out for each subject, using a single, circular 1.9 cm² site in the leading edge of an actively balding area of the vertex as the designated photography site. Total Area Hair Count (TAHC), Target Area Hair Width (TAHW) and Target Area Hair Density/Darkness (TAHD) were assessed via quantitative image analysis to determine non-vellus hair count (primary and secondary endpoint) and total hair count (non-vellus and vellus hair, secondary endpoint). Overall assessment of the vertex was also documented via a global photography procedure as described above prior to any required preparation for macrophotography.

On Day 0, subjects were randomized to receive treatment with the needling device on a weekly (12 treatments), bi-weekly (6 treatments), or monthly (once every 4 weeks, for 3 total treatments) basis for 12 weeks, in a 1:1:1 ratio according to a centrally maintained randomization table. Treatment of the vertex (including transitional areas) with the needling device was initiated on Day 1.

Subjects in the monthly arm received needling device treatment once every 4 weeks, for 3 procedures total (e.g., needling device treatment on Days 1, 29, 57). Subjects in the bi-weekly arm received needling device treatment once every 2 weeks, for 6 procedures total (e.g., needling device treatment on Days 1, 15, 29, 43, 57, and 71). Subjects in the weekly arm received needling device treatment once every week, for 12 procedures total (e.g., needling device treatment on Days 1, 8, 15, 22, 29, 36, 43, 50, 57, 64, 71, and 78).

During needling treatments, the translation speed of the device, which is the speed at which the needling device was glided over the affected skin area, was 2 cm/second, while keeping the device perpendicular to the surface to help achieve a needle strike of 0.8 mm (+/−0.2 mm) micro-needling depth. The device needle array has 12 needles and the number of array oscillations per second was set at 120. The treatment width was 0.9 cm. Two overlapping passes were made in vertical and horizontal directions. As a result, 1600 needle strikes per cm\hat{ }2 were performed. The micro-needling device was glided across the affected area of the skin such that each pass across the area of skin aligned with the prior pass across the skin, without gaps of skin between the passes, in a “mow the lawn” approach. Each pass of the micro-needling device started with a gliding start and a smooth retraction of the device from the skin area following each pass. The micro-needling device was applied to the skin area with consistent, light pressure, allowing the device to glide along the skin. The configuration of needles in the needle array consists of 2 offset rows of 6 microneedles.

Following treatment with the needling device, subjects were permitted to use water and a mild shampoo to clean their hair prior to a post-treatment visit to assess wound closure and skin appearance.

A post-treatment visit was required following each subject's first treatment with the needling device across all study arms to confirm wound closure. The post-treatment visit was performed 24-36 hours following the first treatment with the needling device. If adequate wound closure and skin normality was not achieved, the subject would return for a second post-treatment visit 24-36 hours following the first post-treatment visit. Once adequate wound closure was confirmed, subjects were provided study drug to be applied in accordance with the instructions provided herein (vertex, including transitional areas). The very first application of study drug was performed in-clinic during the first post-treatment visit in which wound closure is confirmed.

The study drug included 2% minoxidil topical solution (MTS) applied topically twice daily for the first four doses following wound closure, followed by 5% MTS applied topically twice daily for subsequent doses until their next scheduled treatment with the needling device. Both were provided in standard sized 60 mL (2 fl. oz.) white-labeled bottles:
Minoxidil topical solution (2%): 20 mg minoxidil per mL, as 60 mL of solution in a 60 mL bottle with the following metered disposable applicators: child-resistant dropper
Minoxidil topical solution (5%): 50 mg minoxidil per mL, as 60 mL of solution in a 60 mL bottle with the following metered disposable applicators: child-resistant dropper. Formulation Including Excipients were as follows: 2% Liquid—Active—2% w/v minoxidil; In active—alcohol, water, propylene glycol; 5% Liquid—Active—5% w/v minoxidil; In active—alcohol, water, propylene glycol.

### 2.4. Storage and Handling

Minoxidil topical solution should was stored at a controlled room temperature of 20-25° C.

Subjects were instructed to make sure that their hair and scalp were completely dry before applying MTS. For each drug application, 1 mL of drug was applied to the area of the scalp being treated (vertex only) using a provided applicator dropper, beginning in the center of the area. The medicine was then massaged into the scalp with fingers. The medicine was applied directly to the scalp. Subjects were instructed to refrain from shampooing their hair for 4 hours after applying MTS. Subjects were also instructed to refrain from using a hairdryer to dry the scalp after applying the MTS. The MTS was allowed to completely dry for 2 to 4 hours after applying it.

This overall study procedure of (1) needling device treatment, (2) 24-hour minimum delay (3) application of 2% MTS twice daily for four doses, (4) application of 5% MTS twice daily until the next needling device treatment, continued per their assigned randomization arm throughout the duration of the study.

Subjects were instructed to apply study drug twice per day per provided instructions with the following exceptions. First, subjects were not to apply MTS on days when treatment with the needling device is scheduled regardless of scheduled procedure time. Second, on post-treatment follow up visits, once wound closure is confirmed, both or one dose of MTS was applied that day if time allowed. So as not to mistakenly remove any of the applied drug, subjects were instructed that hair washing should be done prior to application of study drug, allowing time for the hair and scalp to dry before application.

At treatment Days 29 and 57, in-office visits across all arms also included repeat global photography of overall hair appearance.

Subjects were contacted by phone or assessed during in-office visits on a weekly basis to confirm use of study drug and report any concomitant medication usage or occurrence of any adverse events. The scalp was evaluated for signs of irritation or dermatologic conditions (e.g., erythema, edema, dryness, scaling) at screening and baseline visits as well as prior to any treatment with the needling device. All adverse events were coded and assessed for relation to the study device and study drug.

As an additional safety measure, two blood samples were collected from each subject to measure minoxidil blood serum concentrations: (1) initial 2% MTS application serum level, recorded 1 hour following the first, in-clinic application of 2% MTS, which occurred a minimum of 24 hours after the initial needling device procedure (or as soon thereafter as wound closure is confirmed); and (2) Week X serum level, which was either recorded 1 hour following in-clinic application of 2% MTS, after the treatment session at either (a) 4 weeks or (b) 8 weeks, or (c) during the final study visit at 12 weeks (not time-dependent). The specific value of Week X was assigned to each patient according to a pre-specified randomization plan (1:1:1 randomization to either 4, 8, or 12 weeks).

On Day 85, an in-office visit included repeat hair count analyses (macrophotography) and overall hair appearance (global photography), subject assessment of hair growth, and study-related adverse events. Results were reported by randomization arm.

Baseline (Day 0) and a subject's initial microneedling procedure (Day 1) was permitted to be performed on the same day.

Each study treatment week is a standard period of 7 calendar days, with current nomenclature reflecting the intended day of treatment. Treatment days were scheduled within a window of +/−2 calendar days per treatment week to allow for flexibility in the subject's availability (e.g. Day 8 treatment with the needling device can take place on Day 6 through Day 10).

The overall study treatment phase was a standard period of 85 calendar days (including Baseline Day 0). In consideration of the allowable weekly windows described above, the overall study treatment phase includes a window of +/−6 calendar days. Given the increased frequency of visits for those randomized to the weekly treatment arm, an allowance was made to miss the twelfth and final treatment only if subject could not be scheduled to complete within the allowable overall study treatment phase window. The Day 85 visit in this case remained within its allowable window as described above.

On treatment Days 29 and 57, in-office visits included repeat global photography of overall hair appearance.

### RESULTS

### Safety—Measurement of Minoxidil Serum levels

Previous studies have shown that minimally detectable hemodynamic effects begin to occur at minoxidil serum levels greater than 21.7 ng/mL (Ferry J J, et al. 1996. Hemodynamic Effects of Minoxidil Following Intravenous Infusions in Untreated Hypertensive Patients. Clinical Pharmacology and Therapeutics 59(2):166).

Additionally, studies in which serum levels were measured following routine topical minoxidil application demonstrated average serum levels of 0.7 and 1.7 ng/mL for 2% and 5% minoxidil topical solution (MTS), respectively (U.S. Food and Drug Administration. 1997. Review Memoranda for NDA 20-834 (Pharmacia & Upjohn Company). The FDA has approved both 2% and 5% MTS for over-the-counter use, indicating that minoxidil serum levels of 1.7 ng/mL and less are safe from the FDA's perspective. Furthermore, these levels are well below the level at which systemic effects are minimally detectable (i.e., greater than 21.7 ng/mL in Ferry et al., 1996).

Results from measurements in the current study showed mean serum levels of 1.1 ng/mL (N=33, total 49 observations), as recorded 1 hour after treatment with 2% MTS. This level was expected to be representative of the maximum serum level of minoxidil based on proximity in time to the skin disruption procedure and prior demonstration of maximal topical minoxidil serum levels at 1 hour post administration. Accordingly, the mean observed serum levels were (a) higher than serum levels observed with 2% MTS (0.7 ng/ml), likely due to the changed skin properties from skin disruption while also (b) remaining below the levels observed with 5% MTS (1.7 ng/ml), which have been demonstrated to be safe clinically and in broad over-the-counter usage.

Of note, serum level averages were also calculated at different time periods throughout the study: Day 1, 1.29 ng/mL; Day 29, 0.99 ng/mL; Day 57, 0.79 ng/mL; Day 85, 0.65 ng/mL. These data suggest that there is unlikely to be a meaningful increase in minoxidil serum levels after multiple rounds of treatment. The maximum serum level recorded in the current study was 5.5 ng/mL, which is substantially lower than the MTS 5% maximum level of 16.5 ng/mL observed in one individual in a randomized clinical trial assessing the efficacy of 5% topical minoxidil (Olsen et al. (2002); A randomized clinical trial of 5% topical minoxidil versus 2% topical minoxidil and placebo in the treatment of androgenetic alopecia in men; J Am Acad Dermatol 2002;47:377-85). Again this suggests that the treatment stays within range of what has been previously demonstrated to be safe for over-the-counter minoxidil treatments.

### Efficacy—Assessment of New Hair Growth

To determine efficacy, hair counts were performed on day 0, 29, 57, and 85 and analyzed. 79% of subjects showed an increase in non-vellus target area hair count of more than prior large minoxidil studies described in Olsen 2002 and Olsen 2007. In these earlier minoxidil-only studies, both 5% MTS 5% and 5% minoxidil foam achieved an average of 12% increase above baseline (non-vellus target area hair count) (Olsen 2002 and Olsen 2007). Accordingly, this treatment protocol achieved positive efficacy results relative to minoxidil-only treatments. It was also observed that hair growth increase was greatest in the arm in which skin disruption was performed bi-weekly, as compared to weekly or monthly. The analysis included vellus hair counts, non-vellus hair counts, total hair counts, hair mass, and hair diameter.

### Example 2

A study is conducted to assess the safety and efficacy of treatment methods using a needling device in conjunction with minoxidil in subjects with female pattern hairloss. The study is essentially conducted according to the protocol as described in Example 1 for treatment of adult males with androgenetic alopecia (AGA).

Briefly, the needling device described in Example 1 is used in the study. Macrophotography, global photography, and quantitative image analysis are carried out for each subject in the same manner and on the same days as described above. On Day 0, subjects are randomized to receive treatment with the needling device using the needling method as described (i.e., 1600 needle strikes per cm\hat{ }2) on a weekly (12 treatments; Days 1, 8, 15, 22, 29, 36, 43, 50, 57, 64, 71, and 78), bi-weekly (6 treatments; Days 1, 15, 29, 43, 57, and 71), or monthly (3 treatments; Days 1, 29, 57) basis for 12 weeks, in a 1:1:1 ratio according to a centrally maintained randomization table essentially as described above in Example 1.

Post-needling visits, treatment assessments of wound closure, and patient instructions are performed as described above.

The study drug includes 2% minoxidil topical solution (MTS) applied topically twice daily for the first four doses following wound closure, followed by 5% MTS applied topically twice daily for subsequent doses until their next scheduled treatment with the needling device. Subjects do not apply MTS on days when treatment with the needling device is scheduled.

The scalp is monitored for evaluated for signs of irritation or dermatologic conditions (e.g., erythema, edema, dryness, scaling) at screening and baseline visits as well as prior to any treatment with the needling device. As an additional safety measure, two blood samples are collected from each subject to measure minoxidil blood serum concentrations: (1) initial 2% MTS application serum level, recorded 1 hour following the first, in-clinic application of 2% MTS, which occurrs a minimum of 24 hours after the initial needling device procedure (or as soon thereafter as wound closure is confirmed); and (2) Week X serum level, which is either recorded 1 hour following in-clinic application of 2% MTS, after the treatment session at either (a) 4 weeks or (b) 8 weeks, or (c) during the final study visit at 12 weeks (not time-dependent). The specific value of Week X is assigned to each patient according to a pre-specified randomization plan (1:1:1 randomization to either 4, 8, or 12 weeks).

### Example 3

A study is conducted to assess the safety and efficacy of treatment methods using a needling device in conjunction with minoxidil in subjects with scarring alopecia, e.g., Lichen Planopilaris (LPP).

Skin disruption using a needling device is used in combination with 5% minoxidil topical foam. After wound closure is confirmed following initial treatment with the needling device, subjects are permitted to begin topical application of the minoxidil solution.

Following needling device treatment, 1 mL of generic 5% minoxidil topical foam is applied one time per day in the evening for both male and female subjects. The medications is applied only during weeks in which needling treatment did not occur. The very first application of the medications is performed the week following the first needling treatment.

The hair and scalp are completely dry before applying either medication. For each medication, 1 mL of the medication is applied to the area of the scalp being treated, beginning in the center of the area. The medications are applied directly to the scalp. Subjects are instructed not to shampoo hair for 4 hours after applying either medication. Subjects are also instructed not to use a hairdryer to dry the scalp after applying either medication. Each medication is allowed to completely dry for 2 to 4 hours after applying it, including before going to bed.

After enrollment, the duration of the study is approximately 14 weeks, involving 8 visits. The study consisted of treatments with the needling device every other week for 14 weeks, for 6 treatments total.

On Visit 1, a baseline for each subject is taken. A scalp examination is performed and an active 2 cm hair-bearing target area and a scarred 4 cm target area is identified. The active target area is a 2 cm area considered to be the most active area of LPP for each patient. To mark this target area on the scalp, two 1-2 mm permanent tattoos are made on the scalp by using a disposable 28-gauge needle to inject non-toxic ink into the epidermis of the skin. The scarred target area is an estimated 4 cm squared area located on a scarred area of the scalp (<40% expected hair density in area) chosen by the Investigator. Tattoos are not made in this area, however, standardized photographs are taken of this area. The target areas and top of scalp are photographed. Trichoscopy photography is performed for hair counts. Finally, needling device treatment is performed on both target areas.

On Visit 2, (1 day post-baseline), scalp examination is performed to confirm adequate wound healing. With confirmation of adequate wound healing, subjects receive instructions regarding topical minoxidil solution application.

On each of Visit 3, (2 weeks post-baseline), Visit 5 (6 weeks post-baseline), Visit 6 (8 weeks post-baseline), and Visit 7 (10 weeks post-baseline), needling device treatment is performed on both target areas.

Finally, on Visit 8 (14 weeks post-baseline), a physician performs clinical assessments for each subject, including assessment of active hair-bearing and scarred LPP target areas. Target areas and the top of scalp are photographed, and trichoscopy photography is performed for hair counts.

Levels of minoxidil in serum are measured and the averages calculated to ensure that serum levels remain below the level at which systemic effects are typically observed (>20 ng/mL) (Ferry et al., 1996). Additionally, levels observed with minoxidil-only topical treatments (Olsen et al., 2002 and Olsen et al., 2007) are used as benchmarks. Serum levels are measured and averages calculated at different time periods.

In order to determine efficacy of the study numbers of vellus hair and non-vellus hair are counted and percentage increase in vellus and non-vellus hair is calculated.

### Example 4

A study is conducted to assess the safety and efficacy of treatment methods using a needling device in conjunction with minoxidil in subjects with scarring alopecia, e.g., Lichen Planopilaris (LPP). The study is essentially conducted according to the protocol described in Example 1 for treatment of adult males with androgenetic alopecia (AGA).

Briefly, the needling device described in Example 1 is used in the study. Macrophotography, global photography, and quantitative image analysis are carried out for each subject in the same manner and on the same days as described above. On Day 0, subjects are randomized to receive treatment with the needling device using the needling method as described (i.e., 1600 needle strikes per cm\hat{ }2) on a weekly (12 treatments; Days 1, 8, 15, 22, 29, 36, 43, 50, 57, 64, 71, and 78), bi-weekly (6 treatments; Days 1, 15, 29, 43, 57, and 71), or monthly (3 treatments; Days 1, 29, 57) basis for 12 weeks, in a 1:1:1 ratio according to a centrally maintained randomization table essentially as described above in Example 1.

Post-needling visits, treatment assessments of wound closure, and patient instructions are performed as described above.

The study drug includes 2% minoxidil topical solution (MTS) applied topically twice daily for the first four doses following wound closure, followed by 5% MTS applied topically twice daily for subsequent doses until their next scheduled treatment with the needling device. Subjects do not apply MTS on days when treatment with the needling device is scheduled.

The scalp is monitored for evaluated for signs of irritation or dermatologic conditions (e.g., erythema, edema, dryness, scaling) at screening and baseline visits as well as prior to any treatment with the needling device. As an additional safety measure, two blood samples are collected from each subject to measure minoxidil blood serum concentrations: (1) initial 2% MTS application serum level, recorded 1 hour following the first, in-clinic application of 2% MTS, which occurrs a minimum of 24 hours after the initial needling device procedure (or as soon thereafter as wound closure is confirmed); and (2) Week X serum level, which is either recorded 1 hour following in-clinic application of 2% MTS, after the treatment session at either (a) 4 weeks or (b) 8 weeks, or (c) during the final study visit at 12 weeks (not time-dependent). The specific value of Week X is assigned to each patient according to a pre-specified randomization plan (1:1:1 randomization to either 4, 8, or 12 weeks).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for stimulating hair growth in an area of skin with hair loss comprising:
(i) disrupting the skin in an area of hair loss with a needling device having a needle array and a motor that drives reciprocating needles in the needle array;
(ii) approximately 24 hours following step (i), topically applying 2% minoxidil solution to the disrupted area of the skin 2 times daily for 2 days;
(iii) following step (ii), topically applying 5% minoxidil solution to the disrupted area of the skin 2 times daily for 4 days, for 11 days, or for 25 days; and
(iv) following step (iii), repeating steps (i)-(iii), where topical application of minoxidil solution does not occur on the day of step (i).

2. (canceled)

3. (canceled)

4. The method of claim 1, wherein the minoxidil solution in step (ii) is topically applied approximately every 12 hours.

5. The method of claim 1, wherein the minoxidil solution in step (iii) is topically applied approximately every 12 hours.

6. The method of claim 4, wherein the minoxidil solution in step (iii) is topically applied approximately every 12 hours.

7. The method of claim 1, wherein step (iv) comprises repeating steps (i)-(iii) at least 3 times, at least 6 times, at least 12 times, at least 18 times, or at least 24 times.

8. (canceled)

9. (canceled)

10. (canceled)

11. (canceled)

12. The method of claim 1, wherein step (iv) comprises repeating step (i) 12 to 24 hours following step (iii).

13. The method of claim 1, wherein step (iv) comprises repeating step (i) approximately 24 hours following step (iii).

14. The method of claim 1, wherein the reciprocating needles of the needling device are microneedles.

15. The method of claim 1, wherein the skin is disrupted with the needling device by applying a needle strike density of about 1400 to 1800 needle strikes per cm\hat{ }2.

16. The method of claim 15, wherein the strike density is about 1600 needle strikes per cm\hat{ }2.

17. (canceled)

18. (canceled)

19. (canceled)

20. The method of claim 1, further comprising a step of checking for wound healing after step (i) and prior to step (ii).

21. (canceled)

22. The method of claim 1, wherein the skin is disrupted with the needling device to a skin depth of about 500-1000 μm.

23. (canceled)

24. The method of claim 1, wherein the reciprocating needles in the needling device are set to a depth of 0.8 mm.

25. The method of claim 1, wherein the reciprocating needles in the needling device have a reciprocation frequency of 120 Hz.

26. The method of claim 1, wherein the skin disrupted using the needling device includes an area of skin at least 2 cm beyond a leading edge of the area of hair loss.

27. The method of claim 1, wherein the angle orientation of the needle array is perpendicular to the direction of translation of the needling device over the skin area.

28. The method of claim 1, wherein the needling device is passed over the area of hair loss at a speed of 2 cm/second.

29. The method of claim 1, wherein the needling device is passed over the area of hair loss such that each pass is contiguous to the prior pass without overlap or gaps.

30. The method of claim 1, wherein the needling device is passed over the area of hair loss in a first series of passes parallel to a first direction and in a second series of passes parallel to a second direction that is different than the first direction.

31. (canceled)

32. The method of claim 1, wherein the needles of the needling device form an array of a plurality of lines of needles, each line having a plurality of needles and each line extending parallel to a first direction, wherein the needling device is passed over the area of hair less in a second direction that is perpendicular to the first direction.

33. The method of claim 1, wherein the level of minoxidil in the bloodstream is in the range of 1-4 ng/mL.

34. (canceled)

35. (canceled)

36. The method of claim 1, wherein the method is used to treat a condition associated with hair loss.

37. The method of claim 36, wherein the condition is selected from male pattern baldness, female pattern baldness, androgenic alopecia (AGA), primary cicatricial alopecia (PCA), and lichen planopilaris (LPP).

38. A method for stimulating hair growth in an area of hair loss hair comprising:
(i) disrupting the skin in an area of hair loss with a needling device having a motor that drives reciprocating needles by applying a needle strike density of about 1400 to 1800 needle strikes per cm\hat{ }2;
(ii) approximately 24 hours, 24-36 hours, or 24-48 hours following step (i), topically applying minoxidil solution to the disrupted area of the skin 2 times daily for 6 days;
(iii) repeating steps (i)-(ii).

39. (canceled)

40. (canceled)

41. A method for stimulating hair growth in an area of hair loss hair comprising:
(i) disrupting the skin in an area of hair loss with a needling device having a motor that drives reciprocating needles by applying a needle strike density of about 1400 to 1800 needle strikes per cm\hat{ }2;
(ii) approximately 24 hours. 24-36 hours, or 24-48 hours following step (i), topically applying minoxidil solution to the disrupted area of the skin 2 times daily for 13 days;
(iii) repeating steps (i)-(ii).

42. (canceled)

43. (canceled)

44. A method for stimulating hair growth in an area of hair loss hair comprising:
(i) disrupting the skin in an area of hair loss with a needling device having a motor that drives reciprocating needles by applying a needle strike density of about 1400 to 1800 needle strikes per cm\hat{ }2;
(ii) approximately 24 hours. 24-36 hours, or 24-48 hours following step (i), topically applying minoxidil solution to the disrupted area of the skin 2 times daily for 27 days;
(iii) repeating steps (i)-(ii).

45. (canceled)

46. (canceled)

47. The method of claim 41, wherein the needle strike density is about 1600 needle strikes per cm\hat{ }2.

48 The method of claim 41, wherein the minoxidil solution in step (ii) is topically applied approximately every 12 hours.

49. The method of claim 41, wherein the minoxidil solution in step (ii) is a 5% minoxidil solution.

50. The method of claim 38, wherein the minoxidil solution in step (ii) is topically applied to the disrupted area of the skin as follows:
(a) 2% minoxidil applied 2 times daily for 2 days; and
(b) 5% monoxidil applied 2 times daily for 4 days.

51. The method of claim 41, wherein the minoxidil solution in step (ii) is topically applied to the disrupted area of the skin as follows:
(a) 2% minoxidil applied 2 times daily for 2 days; and
(b) 5% monoxidil applied 2 times daily for 11 days.

53. The method of claim 44, wherein the minoxidil solution in step (ii) is topically applied to the disrupted area of the skin as follows:
(a) 2% minoxidil applied 2 times daily for 2 days; and
(b) 5% monoxidil applied 2 times daily for 25 days.

54. The method of claim 41, wherein step (iii) comprises repeating steps (i)-(ii) at least 3 times, at least 6 times, at least 12 times, at least 18 times, or at least 24 times.

55. (canceled)

56. (canceled)

57. (canceled)

58. (canceled)

59. The method of claim 41, wherein step (iii) comprises repeating step (i) 12 to 24 hours following step (ii).

60. The method of claim 41, wherein step (iii) comprises repeating step (i) approximately 24 hours following step (ii).

61. The method of claim 41, wherein the reciprocating needles of the needling device are microneedles.

62. (canceled)

63. (canceled)

64. (canceled)

65. The method of claim 41, further comprising a step of checking for wound healing after step (i) and prior to step (ii).

66. (canceled)

67. The method of claim 41, wherein the skin is disrupted with the needling device to a skin depth of about 500-1000 μm.

68. (canceled)

69. The method of claim 41, wherein the reciprocating needles in the needling device are set to a depth of 0.8 mm.

70. The method of claim 41, wherein the reciprocating needles in the needling device have a reciprocation frequency of 120 Hz.

71. The method of claim 41, wherein the skin disrupted using the needling device includes an area of skin at least 2 cm beyond a leading edge of the area of hair loss.

72. The method of claim 41, wherein the angle orientation of the needle array is perpendicular to the direction of translation of the needling device over the skin area.

73. The method of claim 41, wherein the needling device is passed over the area of hair loss at a speed of 2 cm/second.

74. The method of 41, wherein the needling device is passed over the area of hair loss such that each pass is contiguous to the prior pass without overlap or gaps.

75. The method of claim 41, wherein the needling device is passed over the area of hair loss in a first series of passes parallel to a first direction and in a second series of passes parallel to a second direction that is different than the first direction.

76. (canceled)

77. The method of claim 41, wherein the needles of the needling device form an array of a plurality of lines of needles, each line having a plurality of needles and each line extending parallel to a first direction, wherein the needling device is passed over the area of hair less in a second direction that is perpendicular to the first direction.

78. The method of claim 41, wherein the level of minoxidil in the bloodstream is in the range of 1-4 ng/mL.

79. (canceled)

80. (canceled)

81. The method of claim 41, wherein the method is used to treat a condition associated with hair loss.

82. The method of claim 81, wherein the condition is selected from male pattern baldness, female pattern baldness, androgenic alopecia (AGA), primary cicatricial alopecia (PCA), and lichen planopilaris (LPP).

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)
